(19) United States
(12) Patent Application Publication
(10) Pub. No.: US 2011/0212127 A1
(43) Pub. Date: Sep. 1, 2011

(54) Method for Preserving Polypeptides Using a Sugar and Polyethyleneimine

(75) Inventors: Jeffrey Drew, (US); Stephen John Ward, (US)

(73) Assignee: Stabilitech Ltd., London (GB)

(21) Appl. No.: 13/076,742

(22) Filed: Mar. 31, 2011

Related U.S. Application Data
(63) Continuation-in-part of application No. 13/120,539, filed on Jun. 7, 2011.

(30) Foreign Application Priority Data
Sep. 24, 2008 (GB) 0817524.2
Sep. 24, 2008 (GB) 0817525.9
Sep. 24, 2008 (GB) 0817526.7
Sep. 24, 2008 (GB) 0817527.5

(51) Int. Cl. A61K 39/12; C07K 1/00; C12N 9/96; A61K 39/02; A61K 39/00; B65D 85/84

(52) U.S. Cl. 424/204.1; 530/345; 435/188; 424/234.1; 424/184.1; 206/524.1

(57) ABSTRACT

The invention relates to the preservation of an active agent, such as a polypeptide, by contacting the active agent with a preservation mixture including a sugar and polyethyleneimine.

### CROSS REFERENCE TO RELATED APPLICATIONS

This application is a continuation-in-part of, and claims priority from, U.S. patent application Ser. No. 13/120,539, filed Mar. 23, 2011, which is the U.S. national phase filing under 35 U.S.C. §371 of PCT international application no. PCT/GB2009/02283, filed Sep. 24, 2009, which claims benefit of Great Britain patent application nos. 0817524.2, filed Sep. 24, 2008, 0817525.9, filed Sep. 24, 2008, 0817526.7, filed Sep. 24, 2008, and 0817527.5, filed Sep. 24, 2008. The contents of the prior applications are incorporated herein by reference.

### FIELD OF THE INVENTION

The invention relates to methods of preserving a polypeptide from thermal degradation and desiccation. The invention also relates to products comprising such preserved polypeptides.

### BACKGROUND TO THE INVENTION

Some biological molecules are sufficiently stable that they can be isolated, purified and then stored in solution at room temperature. However, this is not possible for many materials and techniques involving storage at low temperature, addition of stabilisers, freeze-drying, vacuum-drying and air-drying have been tried to ensure shelf preservation.

Despite the availability of these techniques, some biological materials still show unsatisfactory levels of stability during storage and some techniques lead to added cost and inconvenience. For example, refrigerated transportation and storage is expensive, and any breaks in temperature control can result in reduced efficacy of the biological molecule. Further, refrigerated transport is often not available for the transport of medicines in countries in the developing world.

Also, the stresses of freeze-drying or lyophilisation can be very damaging to some biological materials. Freeze drying of biopharmaceuticals involves freezing solutions or suspensions of thermosensitive biomaterials, followed by primary and secondary drying. The technique is based on sublimation of water at subzero temperature under vacuum without the solution melting. Freeze-drying represents a key step for manufacturing solid protein and vaccine pharmaceuticals. The rate of water vapour diffusion from the frozen biomaterial is very low and therefore the process is time-consuming. Additionally, both the freezing and drying stages introduce stresses that are capable of unfolding or denaturing proteins.

WO 90/05182 describes a method of protecting proteins against denaturation on drying. The method comprises the steps of mixing an aqueous solution of the protein with a soluble cationic polyeletrolyte and a cyclic polyol and removing water from the solution. Diethylaminoethyldextran (DEAE-dextran) and chitosan are the preferred cationic polyelectrolytes, although polyethyleneimine is also mentioned as suitable.

WO-A-2006/0850082 reports a desiccated or preserved product comprising a sugar, a charged material such as a histone protein and a dessication- or thermo-sensitive biological component. The sugar forms an amorphous solid matrix. However, the histone may have immunological consequences if the preserved biological component is administered to a human or animal.

WO 2008/114021 describes a method for preserving viral particles. The method comprises drying an aqueous solution of one or more sugars, a polyethyleneimine and the viral particles to form an amorphous solid matrix comprising the viral particles. The aqueous solution contains the polyethyleneimine at a concentration of 15 μM or less based on the number-average molar mass (Mₙ) of the polyethyleneimine and the sugar concentration or, if more than one sugar is present, total sugar concentration is greater than 0.1M. WO 2008/114021 was published after the priority date of the present application.

### SUMMARY OF THE INVENTION

It has now been found that polypeptide preparations mixed with an aqueous solution containing one, two or more sugars and a polyethyleneimine (PEI) are preserved well on drying such as on freeze-drying. A relatively low concentration of PEI and a relatively high sugar concentration are employed. The polypeptide may be a hormone, growth factor, peptide or cytokine; an antibody or antigen-binding fragment thereof; an enzyme; or a vaccine immunogen. The invention can also be applied to vaccine immunogens such as a subunit vaccine, conjugate vaccine or toxoid.

Accordingly, the present invention provides a method for preserving a polypeptide comprising:
(i) providing an aqueous solution of one or more sugars, a polyethyleneimine and said polypeptide wherein the concentration of polyethyleneimine is 25 μM or less based on the number-average molar mass (Mₙ) of the polyethyleneimine and the sugar concentration or, if more than one sugar is present, total sugar concentration is greater than 0.1M; and
(ii) drying the solution to form an amorphous solid matrix comprising said polypeptide.

The invention further provides:
a dry powder comprising a preserved polypeptide, obtainable by the method of the invention;
a preserved product comprising a polypeptide, one or more sugars and polyethyleneimine, which product is in the form of an amorphous solid;
a sealed vial, ampoule or syringe containing such a dry powder or preserved product; and
use of an excipient comprising:
(a) sucrose, stachyose or raffinose or any combination thereof; and
(b) polyethylenimine at a concentration based on Mₙ of 25 μM or less, for example 5 μM or less;
for the preservation of a polypeptide.

### BRIEF DESCRIPTION OF THE FIGURES

FIG. 1 shows the results obtained in Example 1. The results demonstrate protection of human calcitonin (hCT) from freeze-drying and/or heat treatment, when using an excipient with final concentrations of 1.03M sucrose, 0.09M raffinose and 21 nM PEI (based on an Mₙ of 60,000). FIG. 1 shows the averaged result of detectable hCT as measured by ELISA, after subjecting the samples to the following treatments:
1. Calcitonin resuspended in PBS and frozen
2. Calcitonin resuspended in PBS and freeze dried
3. Calcitonin+sugar mix (sucrose and raffinose) freeze dried
4. Calcitonin+sugar mix (sucrose and raffinose) freeze dried+heated
5. Calcitonin+excipient (preservation mixture composed of sucrose, raffinose and PEI) freeze dried (invention)
6. Calcitonin+excipient (preservation mixture composed of sucrose, raffinose and PEI) freeze dried and heat treated (invention)

FIG. 2 shows the results obtained in Example 2. The ability of a preservation mixture (excipient) according to the invention to stabilize G-CSF against heat treatment was assessed by monitoring the ability of G-CSF to stimulate ERK1/2 phosphorylation. HL60 cells were serum starved for 24 hours and then stimulated for 5 minutes with the treatments indicated (100 ng/ml G-CSF). Whole cell extracts were resolved by SDS-PAGE and then transferred to nylon membranes, which were immunoprobed with antibodies against phosphorylated and total ERK1/2.
Panel A shows: Control (serum starved+PBS), UT G-CSF (untreated G-CSF) and freeze thaw G-CSF (standard G-CSF mixed with excipient and frozen) samples.
Panel B shows: Control (serum starved+PBS), UT G-CSF (untreated G-CSF) and Excipient/HT G-CSF (G-CSF mixed with excipient then heated) samples.
Panel C shows: Control (serum starved+PBS), UT G-CSF (untreated G-CSF) and G-CSF Excipient/FD (G-CSF mixed with excipient and freeze dried) samples.
Panel D shows: Control (serum starved+PBS), UT G-CSF (untreated G-CSF) and G-CSF Excipient/FD/HT (G-CSF mixed with excipient, freeze dried and heat treated) samples.

FIG. 3 depicts the results from Example 3. The residual activity of anti-human tumor necrosis factor-α antibodies (rat monoclonal anti-TNFα, Invitrogen Catalogue No.: SKU#RHTNFA00) was assessed in an ELISA after the indicated treatment:
1. anti-hTNFα rat mAb (test)—no treatment+PBS (4° C.)
2. anti-hTNFα rat mAb—freeze dried+excipient and stored at 4° C.
3. anti-hTNFα rat mAb—freeze dried+excipient and heat treated at 65° C. for 24 hours
4. anti-hTNFα rat mAb—heat treated+PBS at 65° C. for 24 hours

The excipient contained a final concentration of 0.91M sucrose, 0.125M raffinose and 25 nM PEI (based on Mₙ of 60,000). The results show that the inclusion of excipient prior to freeze drying of the antibody enabled the said antibody to withstand to a significantly higher level, heat challenge for significantly longer periods.

FIG. 4 shows the preservation of luciferase in Example 4 after freezing and then freeze-drying overnight, in an excipient (preservation mixture) containing a final concentration of 1.092M sucrose, 0.0499M stachyose and either 27 nM, 2.7 nM and 0.27 nM PEI (Sigma catalogue number P3143, Mₙ 60,000). As can be clearly seen, there is improved thermal stability of Luciferase when dried in the presence of the excipient.

FIG. 5 shows the preservation of beta-galactosidase activity in Example 5 following freeze-drying in an excipient (preservation mixture) containing a final concentration of 0.97 M sucrose, 0.13M raffinose and 14M, 2.6 μM, 0.26 μM, 26 nM or 2.6 nM PEI (Sigma catalogue number P3143, Mₙ 60,000). This Example clearly demonstrates that there is significant improvement in the thermal stability of beta-galactosidase when dried in the excipient.

FIG. 6 shows the results of the experiment of Example 6 evaluating a range of excipients to provide thermostabilisation of anti-human TNFα antibody. Samples of antibody in excipient containing various concentrations of sucrose (Suc), raffinose (Raf) and PEI were freeze-dried and then heated at 45° C. for 1 week.

FIG. 7 shows the effects of excipient composition on the amount of anti-TNFα measured after freeze-drying (FD) in Example 7. HPLC peak areas are depicted. No antibody was measured when freeze-dried in PBS. A significant amount of anti-TNFα antibody was lost when freeze-dried in sugars alone. A much greater amount of anti-TNFα was measured when the antibody was freeze-dried with sugars and PEI.

FIG. 8 depicts the result of the experiment of Example 8. Anti-TNFα antibody was freeze-dried in 1M sugar (0.9M sucrose and 0.1M raffinose) and 0.0025 nM PEI.

FIG. 9 compares the thermal stability of freeze-dried influenza haemagglutinin (HA) against liquid control samples (Liquid PBS) as tested in Example 9. Samples of HA protein were prepared in PBS or an excipient mixture of 1M sucrose/100 mM raffinose/16.6 nM PEI (based on Mn). The mixture was then lyophilised (FD), secondary drying being carried out between −32° C. and 20° C. over a 3 day cycle. After lyophilisation, one of the samples was thermally challenged at 80° C. for 1 hour (FD HT excipient).

FIG. 10 shows the effects of sugars and PEI on luciferase freeze-dried with bovine serum albumin (BSA) in Example 10. This six-part Figure shows the effects on luciferase activity of sugar mix (sm) and PEI—alone and together—when added before or after freeze-drying (FD). Prior to analysis, freeze-dried samples were held at 45° C. for 2 weeks, then at room temperature for a further 2 weeks. Error bars shown are standard error of the mean.

FIG. 11 shows the effect of freezing β-gal in the presence of sugar/PEI excipients as reported in Example 11. Following freeze-drying, β-gal activity was high in sucrose/raffinose excipients compared to PBS. The presence of PEI at 13.3 μM in combination with sucrose/raffinose further enhanced enzyme activity compared to sucrose/raffinose alone. Error bars show standard error of the mean.

FIG. 12 shows the results obtained in Example 12 of subjecting samples of horse radish peroxidase (HRP) to freeze-drying and then 2, 4 or 6 heat-freeze cycles by removing them from the −20° C. freezer and placing them in an incubator at 37° C. for 4 hours before replacing them in the freezer for 20 hours 2, 4 or 6 times. The results show for all treatments and storage conditions that HRP activity is better maintained in the presence of sucrose, raffinose either with or without PEI, than PBS alone. However, the presence of sugars in combination with PEI at the initial freeze-drying stage significantly reduces loss of HRP activity.

FIG. 13 depicts the results obtained in Example 13. The activity of wet, dried and freeze-dried alcohol oxidase in the presence and absence of excipients is shown:
D0 to D16: days incubated at 37° C. (for dried and freeze-dried samples);
No MeOH: no methanol added (negative control);
wet: samples stored and tested with desiccation (i.e. fresh);
FD: freeze-dried;
D: dried;
G1&G2: excipient mix conditions Gibson 1 & 2 respectively according to Example 10 of WO 90/05182; and
S1 and S2: excipient mix conditions Stabilitech 1 and 2 respectively according to the present invention.

FIG. 14 shows an assessment of the level of phosphorylated ERK1/ERK2 in HL-60 cells induced by recombinant human G-CSF in Example 14. G-CSF was mixed with an excipient containing sucrose, raffinose and PEI, then freeze dried (FD) and heat treated at 56° C. (HT).

FIG. 15 shows the recovery of IgM in Example 15 after freeze-drying in various excipients and thermal challenge. The error bars represent standard error.

FIG. 16 shows the level of phosphorylated ERK1/ERK2 in HL-60 cells induced by recombinant human G-CSF in Example 16. G-CSF was mixed with an excipient containing sucrose, raffinose and PEI, then freeze dried (FD) and heat treated at 37° C. or 56° C. (HT).

FIG. 17 shows the initial thermal challenge study in Example 17. TC denotes thermal challenge. The error bars show the standard deviation, n=2.

FIG. 18 shows the residual F(ab′)2 activity (at 2 μg·ml) remaining in Example 17 at 24 hours, 5 days and 7 days following thermal challenge at +56° C.

### DETAILED DESCRIPTION OF THE INVENTION

### Summary

The present invention relates to the preservation of an active agent by contacting the active agent with a preservation mixture. The active agent may be a polypeptide such as a hormone, growth factor, peptide or cytokine; an antibody or antigen-binding fragment thereof; or an enzyme. The active agent may be a vaccine immunogen such as a subunit vaccine, conjugate vaccine or toxoid.

The preservation mixture is an aqueous solution of PEI and one, two or more sugars. Low concentrations of PEI and relatively high concentrations of sugar are used. The resulting solution in which the active agent is present is then dried to form an amorphous solid matrix comprising the active agent. The matrix is storage stable at ambient temperature. If an aqueous solution comprising the active agent is required for administration, it is reconstituted from the solid matrix immediately prior to use.

The invention thus enables the structure and function of the active agent to be preserved during the drying step and storage. Biological activity of the active agent following drying can thus be maintained. The preserved active agent demonstrates improved thermal and desiccation resistance allowing extension of shelf life, ease of storage and transport and obviating the need for a cold chain for distribution. The preservation mixture can thus provide protection as a cryoprotectant (protection against freeze damage), lyoprotectant (protection against desiccation) and/or a thermoprotectant (protection against temperatures higher or lower than 4° C.).

### Polypeptides

Any polypeptide is suitable for use in the invention. For example, the polypeptide may be a small peptide of less than 15 amino acids such as 6 to 14 amino acids (e.g. oxytocin, cyclosporin), a larger peptide of between 15 and 50 amino acids (e.g. calcitonin, growth hormone releasing hormone 1-29 (GHRH)), a small protein of between 50 and 250 amino acids in length (e.g. insulin, human growth hormone), a larger protein of greater than 250 amino acids in length or a multisubunit protein comprising a complex of two or more polypeptide chains. The polypeptide may be a peptide hormone, growth factor or cytokine. It may be an antigen-binding polypeptide, receptor inhibitor, ligand mimic or receptor blocking agent. Typically, the polypeptide is in substantially pure form. It may thus be an isolated polypeptide. For example, the polypeptide may be isolated following recombinant production.

For example, the polypeptide may be a hormone selected from a growth hormone (GH), prolactin (PRL), a human placental lactogen (hPL), a gonadotrophin (e.g. lutenising hormone, follicle stimulating hormone), a thyroid stimulating hormone (TSH), a member of the pro-opiomelanocortin (POMC) family, vasopressin and oxytocin, a natriuretic hormone, parathyroid hormone (PTH), calcitonin, insulin, a glucagon, somatostatin and a gastrointestinal hormone.

The polypeptide may be a Tachykinin peptide (e.g. Substance P, Kassinin, Neurokinin A, Eledoisin, Neurokinin B), a vasoactive intestinal peptide (e.g. VIP (Vasoactive Intestinal Peptide; PHM27), PACAP (Pituitary Adenylate Cyclase Activating Peptide), Peptide PHI 27 (Peptide Histidine Isoleucine 27), GHRH 1-24 (Growth Hormone Releasing Hormone 1-24), Glucagon, Secretin), a pancreatic polypeptide-related peptide (e.g. NPY, PYY (Peptide YY), APP (Avian Pancreatic Polypeptide), PPY (Pancreatic Polypeptide), an opioid peptide (e.g. Proopiomelanocortin (POMC) peptides, Enkephalin pentapeptides, Prodynorphin peptide, a calcitonin peptide (e.g. Calcitonin, Amylin, AGG01) or another peptide (e.g. B-type Natriuretic Peptide (BNP)).

The polypeptide may be a growth factor selected from a member of the epidermal growth factor (EGF) family, platelet-derived growth factor family (PDGF), fibroblast growth factor family (FGF), Transforming Growth Factors-β family (TGFs-β), Transforming Growth Factor-α (TGF-α), Erythropoietin (Epo), Insulin-Like Growth Factor-I (IGF-I), Insulin-Like Growth Factor-II (IGF-II). Typically, the growth factor is a Transforming growth factor beta (TGF-β), a Nerve growth factor (NGF), a Neurotrophin, a Platelet-derived growth factor (PDGF), Erythropoietin (EPO), Thrombopoietin (TPO), Myostatin (GDF-8), a Growth differentiation factor-9 (GDF9), Acidic fibroblast growth factor (aFGF or FGF-1), Basic fibroblast growth factor (bFGF or FGF-2), Epidermal growth factor (EGF) or a Hepatocyte growth factor (HGF).

The polypeptide may be a cytokine selected from Interleukin-1 (IL-1), Interleukin-2 (IL-2), Interleukin-6 (IL-6) Interleukin-8 (IL-8), Tumor Necrosis Factor-α (TNF-α), Tumor Necrosis Factor-β (TNF-β), Interferon-γ (INF-γ) and a Colony Stimulating Factor (CSF). Typically the cytokine is a Granulocyte-colony stimulating factor (G-CSF) or a Granulocyte-macrophage colony stimulating factor (GM-CSF).

The polypeptide may be a blood-clotting factor such as Factor VIII, Factor V, von Willebrand factor or coagulation factor III.

### Antibodies

An antibody for use in the invention may either be a whole antibody or an antigen-binding fragment thereof.

### Whole Antibodies

In one embodiment, the antibody is an immunoglobulin (Ig) monomer, dimer, tetramer, pentamer, or other oligomer. Each antibody monomer may comprise four polypeptide chains (for example, a conventional antibody consisting of two identical heavy chains and two identical light chains). Alternatively, each antibody monomer consists of two polypeptide chains (for example, a heavy chain antibody consisting of two identical heavy chains).

The antibody can be any class or isotype of antibody (for example IgG, IgM, IgA, IgD or IgE) or any subclass of antibody (for example IgG subclasses IgG1, IgG2, IgG3, IgG4 or IgA subclasses IgA1 or IgA2). Typically, the antibody is an IgG such as an IgG1, IgG2 or IgG4 antibody. Usually, the antibody is an IgG1 or IgG2 antibody.

Typically the antibody or antigen-binding fragment is of mammalian origin. The antibody may thus be a primate, human, rodent (e.g. mouse or rat), rabbit, ovine, porcine, equine or camelidae antibody or antibody fragment. The antibody or antibody fragment may be of shark origin.

The antibody may be a monoclonal or polyclonal antibody. Monoclonal antibodies are obtained from a population of substantially homogenous antibodies that are directed against a single determinant on the antigen. A population of polyclonal antibodies comprises a mixture of antibodies directed against different epitopes.

### Antigen-Binding Fragments

The antigen-binding fragment can be any fragment of an antibody which retains antigen-binding ability, for example a Fab, F(Ab')₂, Fv, disulphide-linked Fv, single chain Fv (scFv), disulphide-linked scFv, diabody, linear antibody, domain antibody or multispecific antibody. Such fragments comprise one or more antigen binding sites. In one embodiment, the antigen-binding fragment comprises four framework regions (e.g. FR1, FR2, FR3 and FR4) and three complementarity-determining regions (e.g. CDR1, CDR2 and CDR3). Methods suitable for detecting ability of a fragment to bind an antigen are described herein and are well known in the art, for example immunoassays and phage display.

The antibody binding fragment may be a monospecific, bispecific or multispecific antibody. A multispecific antibody has binding specificity for at least one, at least two, at least three, at least four or more different epitopes or antigens. A bispecific antibody is able to bind to two different epitopes or antigens. For example, a bispecific antibody may comprise two pairs of V_{H} and V_{L}, each V_{H}/V_{L} pair binding to a single antigen or epitope. Methods for preparing bispecific antibodies are known in the art, for example involving coexpression of two immunoglobulin heavy chain-light chain pairs, fusion of antibody variable domains with the desired binding specificities to immunoglobulin contant domain sequences, or chemical linkage of antibody fragments.

The bispecific antibody “diabody” comprises a heavy chain variable domain connected to a light chain variable domain in the same polypeptide chain (V_{H}-V_{L}). Diabodies can be generated using a linker (e.g. a peptide linker) that is too short to allow pairing between the two domains on the same chain, so that the domains are forced to pair with the complementary domains of another chain and create a dimeric molecule with two antigen-binding sites.

A suitable scFv antibody fragment may comprise V_{H} and V_{L}, domains of an antibody wherein these domains are present in a single polypeptide chain. Generally, the Fv polypeptide further comprises a polypeptide linker between the V_{H} and V_{L} domains, which enables the scFv to form the desired structure for antigen binding.

A domain antibody for use in the methods of the invention may essentially consist of a light chain variable domain (e.g. a V_{L}) or of a heavy chain variable domain (e.g. a V_{H}). The heavy chain variable domain may be derived from a conventional four-chain antibody or from a heavy chain antibody (e.g. a camelidae V_{HH}).

### Modifications

The whole antibody or fragment thereof may be associated with other moieties, such as linkers, which may be used to join together two or more fragments or antibodies. Such linkers may be chemical linkers or can be present in the form of a fusion protein with a fragment or whole antibody. The linkers may thus be used to join together whole antibodies or fragments, which have the same or different binding specificities.

In a further embodiment, the antibody or antigen-binding fragment is linked to a further moiety such as a toxin, therapeutic drug (e.g. chemotherapeutic drug), radioisotope, liposome or prodrug-activating enzyme. The type of further moiety will depend on the end use of the antibody or antigen-binding fragment.

The antibody or antigen-binding fragment may be linked to one or more small molecule toxins (e.g. calicheamicin, maytansine, trichothene and CC1065) or an enzymatically active toxin or fragment thereof (e.g. diphtheria toxin, exotoxin A chain from Pseudomonas aeruginosa, ricin A chain, abrin A chain, modeccin A chain, alpha-sarcin, Aleurites fordii proteins, dianthin proteins, curcin, crotin, gelonin, mitogellin, restrictocin, phenomycin, enomycin or tricothecenes).

Radioisotopes suitable for linking to the antibody or antigen-binding fragments include, but are not limited to Tc⁹⁹, At²¹¹, I¹³¹, I¹²⁵, Y⁹⁰, Re¹⁸⁶, Re¹⁸⁸, Sm¹⁵³, Bi²¹² and P³².

The antibody or antigen-binding fragment may be linked for example, to a prodrug-activating enzyme that converts or is capable of converting a prodrug to an active anti-cancer drug. For example, alkaline phosphatase can be used to convert phosphate-containing prodrugs into free drugs, arylsufatase may be used to convert sulfate-containing prodrugs into free drugs, cytosine deaminase may be used to convert non-toxic 5-fluorocytosine into the anti-cancer drug 5-fluorouracil; and proteases such as serratia protease, thermolysin, subtilisin, carboxypeptidases and cathepsins are useful for converting peptide-containing prodrugs into free drugs. The enzyme may be a nitroreductase which has been identified as useful in the metabolism of a number of prodrugs in anti-cancer gene therapy. Alternatively, antibodies or antigen-binding fragments with enzymatic activity can be used to convert prodrugs into free active drugs.

A suitable chemotherapeutic agent may include, but is not limited to an alkylating agent such as thiotepa and cyclosphosphamide; an alkyl sulfonate such as busulfan, improsulfan and piposulfan; an aziridine such as benzodopa, carboquone, meturedopa and uredopa; a nitrogen mustard such as chlorambucil, chlornaphazine, ifosfamide, melphalan; a nitrosurea such as carmustin and fotemustine; an anti-metabolite such as methotrexate and 5-fluorouracil (5-FU); a folic acid analogue such as denopterin and pteropterin; a purine analogue such as fludarabine and thiamiprine; a pyrimidine analogue such as ancitabine, azacitidine, carmofur and doxifluridine; a taxoid such as paclitaxel and doxetaxel; and pharmaceutically acceptable salts, acids or derivatives of any of the above.

In another embodiment, the antibody or antibody fragment may be PEGylated. Thus, one or more polyethylene glycol molecules may be covalently attached to the antibody molecule or antibody fragment molecule. From one to three polyethylene glycol molecules may be covalently attached to each antibody molecule or antibody fragment molecule. Such PEGylation is predominantly used to reduce the immunogenicity of an antibody or antibody fragment and/or increase the circulating half-life of the antibody or antibody fragment.

### Chimeric, Humanized or Human Antibodies

In one embodiment the antibody or antigen-binding fragment is a chimeric antibody or fragment thereof comprising sequence from different natural antibodies. For example, the chimeric antibody or antigen-binding fragment may comprise a portion of the heavy and/or light chain identical or homologous to corresponding sequences in antibodies of a particular species or antibody class, while the remainder of the chain is identical or homologous to corresponding sequences in antibodies of another species or antibody class. Typically, the chimeric antibody or antigen-binding fragment comprises a chimera of mouse and human antibody components.

Humanized forms of non-human antibodies are chimeric antibodies that contain minimal sequence derived from non-human immunoglobulin. A suitable humanized antibody or antigen-binding fragment may comprise for example, immunoglobulin in which residues from a hypervariable region (e.g. derived from a CDR) of the recipient antibody or antigen-binding fragment are replaced by residues from a hypervariable region of a non-human species (donor antibody) such as mouse, rat, rabbit or non-human primate having the desired specificity, affinity and/or capacity. In some instances, some framework region residues of the human immunoglobulin may be replaced by corresponding non-human residues.

As an alternative to humanization, human antibodies or antigen-binding fragments can be generated. For example, transgenic animals (e.g. mice) can be produced that are capable, upon immunization, of producing a full repertoire of human antibodies in the absence of endogenous immunoglobulin production. For example, homozygous deletion of the antibody heavy-chain joining region (J_{H}) gene in chimeric and germ-line mutant mice can result in complete inhibition of endogenous antibody production. Human germ-line immunoglobulin genes can be transferred to such germ-line mutant mice to result in the production of human antibodies upon antigen challenge. A human antibody or antigen-binding fragment can also be generated in vitro using the phage display technique.

### Targets

An antibody or antigen-binding fragment capable of binding any target antigen is suitable for use in the methods of the present invention. The antibody or antigen-binding fragment may be capable of binding to an antigen associated with an autoimmune disorder (e.g. Type I diabetes, multiple sclerosis, rheumatoid arthritis, systemic lupus erythematosus, Crohn's disease and myasthenia gravis), an antigen associated with a cancer or an inflammatory state, an antigen associated with osteoporosis, an antigen associated with Alzheimer's disease, or a bacterial or viral antigen.

In particular, the target to which an antibody or antigen-binding fragment may bind can be a CD antigen, growth factor, growth factor receptor, cell surface receptor such as an apoptosis receptor, a protein kinase or an oncoprotein. The antibody or antigen-binding fragment, for example a chimeric, humanized or human IgG1, IgG2 or IgG4 monoclonal antibody or antibody fragment, may thus be capable of binding to tumour necrosis factor α (TNF-α), interleukin-2 (IL-2), interleukin-6 (IL-6), glycoprotein IIb/IIIa, CD33, CD52, CD20, CD11a, CD3, RSV F protein, HER2/neu (erbB2) receptor, vascular endothelial growth factor (VEGF), epidermal growth factor receptor (EGFR), anti-TRAILR2 (anti-tumour necrosis factor-related apoptosis-inducing ligand receptor 2), complement system protein C5, α4 integrin or IgE.

More specifically, in the context of anti-cancer monoclonal antibodies, the antibody or antigen-binding fragment may be an antibody or antibody fragment capable of binding to epithelial cell adhesion molecule (EpCAM), mucin-1 (MUC1/Can-Ag), EGFR, CD20, carcinoembryonic antigen (CEA), HER2, CD22, CD33, Lewis Y and prostate-specific membrane antigen (PMSA). Again, the antibody is typically a chimeric, humanized or human IgG1, IgG2 or IgG4 monoclonal antibody.

Suitable monoclonal antibodies include, but are not limited to: infliximab (chimeric antibody, anti-TNFα), adalimumab (human antibody, anti-TNFα), basiliximab (chimeric antibody, anti-IL-2), abciximab (chimeric antibody, anti-GpIIb/IIIa), daclizumab (humanized antibody, anti-IL-2), gemtuzumab (humanized antibody, anti-CD33), alemtuzumab (humanized antibody, anti-CD52), edrecolomab (murine Ig2a, anti-EpCAM), rituximab (chimeric antibody, anti-CD20), palivizumab (humanized antibody, RSV target), trastuzumab (humanized antibody, anti-HER2/neu(erbB2) receptor), bevacizumab (humanized antibody, anti-VEGF), cetuximab (chimeric antibody, anti-EGFR), eculizumab (humanized antibody, anti-complement system protein C5), efalizumab (humanized antibody, anti-CD11a), ibritumomab (murine antibody, anti-CD20), muromonab-CD3 (murine antibody, anti-T cell CD3 receptor), natalizumab (humanized antibody, anti-α 4 integrin), nimotuzumab (humanized IgG1, anti-EGF receptor), omalizumab (humanized antibody, anti-IgE), panitumumab (human antibody, anti-EGFR), ranibizumab (humanized antibody, anti-VEGF), ranibizumab (humanized antibody, anti-VEGF) and I-131 tositumomab (humanized antibody, anti-CD20).

### Preparation of Antibodies

Suitable monoclonal antibodies may be obtained for example, by the hybridoma method (e.g. as first described by Kohler et al Nature 256:495 (1975)), by recombinant DNA methods and/or following isolation from phage or other antibody libraries.

The hybridoma technique involves immunisation of a host animal (e.g. mouse, hamster or monkey) with a desired immunogen to elicit lymphocytes that produce or are capable of producing antibodies that specifically bind to the immunogen. Alternatively, lymphocytes may be immunized in vitro. Lymphocytes are then fused with myeloma cells using a suitable fusing agent, such as polyethylene glycol, to form a hybridoma cell.

An antibody or antibody fragment can also be isolated from antibody phage libraries as an alternative to traditional monoclonal antibody hybridoma techniques for isolation of monoclonal antibodies. In particular, phage display may be used to identify antigen-binding fragments for use in the methods of the invention. By using phage display for the high-throughput screening of antigen-antibody binding interactions, antigen-binding fragments displayed on phage coat proteins can be isolated from a phage display library. By immobilising a target antigen on a solid support, a phage that displays an antibody capable of binding that antigen will remain on the support while others can be removed by washing. Those phages that remain bound can then be eluted and isolated, for example after repeated cycles of selection or panning. Phage eluted in the final selection can be used to infect a suitable bacterial host from which phagemids can be collected and the relevant DNA sequence excised and sequenced to identify the relevant antigen-binding fragment.

Polyclonal antiserum containing the desired antibodies is isolated from animals using techniques well known in the art. Animals such as sheep, rabbits or goats may be used for example, for the generation of antibodies against an antigen of interest by the injection of this antigen (immunogen) into the animal, sometimes after multiple injections. After collection of antiserum, antibodies may be purified using immunosorbent purification or other techniques known in the art.

The antibody or antigen-binding fragment used in the method of the invention may be produced recombinantly from naturally occurring nucleotide sequences or synthetic sequences. Such sequences may for example be isolated by PCR from a suitable naturally occurring template (e.g. DNA or RNA isolated from a cell), nucleotide sequences isolated from a library (e.g. an expression library), nucleotide sequences prepared by introducing mutations into a naturally occurring nucleotide sequence (using any suitable technique known, e.g. mismatch PCR), nucleotide sequence prepared by PCR using overlapping primers, or nucleotide sequences that have been prepared using techniques for DNA synthesis. Techniques such as affinity maturation (for example, starting from synthetic, random or naturally occurring immunoglobulin sequences), CDR grafting, veneering, combining fragments derived from different immunoglobulin sequences, and other techniques for engineering immunoglobulin sequences may also be used.

Such nucleotide sequences of interest may be used in vitro or in vivo in the production of an antibody or antigen-binding fragment for use in the invention, in accordance with techniques well known to those skilled in the art.

For recombinant production of a monoclonal antibody or antigen-binding fragment, the nucleic acid encoding it is isolated and inserted into a replicable vector for further cloning or for expression. The vector components generally including, but is not limited to one or more of the following: a signal sequence, an origin of replication, one or more marker genes, an enhancer element, a promoter, and a transcription termination sequence. Suitable host cells for cloning or expressing the DNA in the vectors are prokaryote, yeast, or higher eukaryote cells such as E. coli and mammalian cells such as CHO cells. Suitable host cells for the expression of glycosylated antibody are derived from multi-cellular organisms. Host cells are transformed with the expression or cloning vectors for antibody production and cultured in conventional nutrient media modified as appropriate for inducing promoters, selecting transformants, or amplifying the genes encoding the desired sequences.

When using recombinant techniques, the antibody can be produced intracellularly or directly secreted into the medium. If the antibody is produced intracellularly, as a first step, the particulate debris of either host cells or lysed cells, is removed, for example by centrifugation or ultra filtration. Where the antibody is secreted into the medium, supernatants from expression systems are generally first concentrated using a commercially available protein concentration filter. The antibody composition prepared from the cells can be purified using, for example, hydyoxylapatite chromatography, gel electrophoresis, dialysis and affinity chromatography.

The purified antibodies may then be isolated and optionally made into antigen-binding fragments and/or derivatised.

### Enzymes

Any protein enzyme is suitable for use in the invention. Such an enzyme comprises an active site and is capable of binding a substrate. The enzyme may be a monomer consisting of one polypeptide chain. Alternatively, the enzyme may be a dimer, tetramer or oligomer consisting of multiple polypeptide chains. The dimer, tetramer or oligomer may be a homo- or hetero-dimer, tetramer or oligomer respectively. For example, the enzyme may need to form an aggregate (e.g. a dimer, tetramer or oligomer) before full biological activity or enzyme function is conferred. The enzyme may be an allosteric enzyme, an apoenzyme or a holoenzyme.

The enzyme may be conjugated to another moiety (e.g. a ligand, antibody, carbohydrate, effector molecule, or protein fusion partner) and/or bound to one or more cofactors (e.g. coenzyme or prosthetic group).

The moiety to which the enzyme is conjugated may include lectin, avidin, a metabolite, a hormone, a nucleotide sequence, a steroid, a glycoprotein, a glycolipid, or any derivative of these components.

Cofactors include inorganic compounds (e.g. metal irons such as iron, manganese, cobalt, copper, zinc, selenium, molybdenum) or organic compounds (e.g. flavin or heme). Suitable coenzymes include riboflavin, thiamine, folic acid which may carry hydride iron (H⁻) carried by NAD or NADP⁺, the acetyl group carried by coenzyme A, formyl, methenyl or methyl groups carried by folic acid and the methyl group carried by S-adenosyl methionine.

In another embodiment, the enzyme may be PEGylated especially if the enzyme is a therapeutic enzyme that is administered to a patient. Thus, one or more polyethylene glycol molecules may be covalently attached to the enzyme molecule. From one to three polyethylene glycol molecules may be covalently attached to each enzyme molecule. Such PEGylation is predominantly used to reduce the immunogenicity of an enzyme and/or increase the circulating half-life of the enzyme.

A suitable enzyme includes any enzyme classified under the International Union of Biochemistry and Molecular Biology Enzyme classification system of EC numbers including an oxidoreductase (EC 1), a transferase (EC 2), a hydrolase (EC 3), a lyase (EC 4), an isomerase (EC 5) or a ligase (EC 6). A typical enzyme is any enzyme that is used industrially.

An enzyme that is specific for any type of substrate is suitable for use in the present invention. Examples of a suitable enzyme includes a α-galactosidase, β-galactosidase, luciferase, serine proteinase, endopeptidase (e.g. cysteine endopeptidase), caspase, chymase, chymotrypsin, endopeptidase, granzyme, papain, pancreatic elastase, oryzin, plasmin, renin, subtilisin, thrombin, trypsin, tryptase, urokinase, amylase (e.g. α-amylase), xylanase, lipase, transglutaminase, cell-wall-degrading enzyme, glucanase (e.g. β-glucanase), glucoamylase, coagulating enzyme, milk protein hydrolysate, cell-wall degrading enzyme, blood coagulating enzyme, hementin, lysozyme, fibre-degrading enzyme, phytase, cellulase, hemicellulase, polymerase, protease, mannanase or glucoamylase.

An enzyme preserved according to the invention may thus be a therapeutic enzyme that is used to treat a disease or other medical condition, an enzyme used in industry for the production of bulk products such as glucose or fructose, in food processing and food analysis, in laundry and automatic dishwashing detergents, in the textile, pulp, paper and animal feed industries, as a catalyst in synthesis or fine chemicals, in diagnostic applications such as in clinical diagnosis, in biosensors or in genetic engineering.

Therapeutic enzymes to which the present invention can be applied include:
a DNAase, for example a recombinant DNAase I such as Pulmozyme or Dornase that cleaves the DNA in the pulmonary mucus of children having cystic fibrosis;
a gastric lipase such as Meripase which is a recombinant mammalian gastric lipase for the treatment of lipid malabsorption related to exocrine pancreatic lipase insufficiency;
a mannose-terminated glucocerebrosidase such as Cerezyme which is a recombinant mannose-terminated glucocerebrosidase for the treatment of Gaucher disease, an inherited disorder that is caused by a deficiency in the enzyme glucocerebrosidase;
α-galactosidase which is used in the treatment of the related glycogen storage disease Fabry disease;
an adenosine deaminase (ADA) such as Pegademase that is used to treat ADA deficiency, a severe combined immunodeficiency;
a phenylalanine ammonia lyase such as the PEGylated recombinant phenylalanine ammonia lyase Kuvan that is used for the treatment of phenylketonuria;
tissue plasminogen activator, urokinase and streptokinase which are used in blood fibrinolysis to treat blood clots;
a urate oxidase such as Elitek (rasburicase) which is a recombinant urate-oxidase that is produced by a genetically modified yeast and that is used in the treatment or prophylaxis of hyperuricemia in patients with leukaemia or lymphoma;
L-asparaginase which is used in the treatment of childhood acute lymphoblastic leukaemia;
Factor VIIa, used by patients with hemophilia;
Factor IX which is used in the treatment of hemophilia B; and
a superoxide dismutase such as the bovine superoxide dismutase Orgotein that is used for the treatment of familial amyotrophic lateral sclerosis.

Enzymes for use in food applications such as baking include amylases, xylanases, oxidoreductases, lipases, proteases and transglutaminase. Enzymes for use in fruit juice production and fruit processing include cell-wall-degrading enzymes. Enzymes for use in brewing include bacterial α-amylase, β-glucanase and glucoamylase in mashing, fungal α-amylase in fermentation and cysteine endopeptidase in post fermentation. Enzymes for use in dairy applications include coagulating enzymes, lipase, lysozyme, milk protein hydrolysates, transglutaninase, and β-galactosidase. Enzymes for use in detergent compositions include proteases, amylases, lipases, cellulases and mannanase. Enzymes for use in animal feed include fibre-degrading enzymes, phytases, proteases and amylases. Enzymes for use in pulp and paper processing include cellulases and hemicellulases.

The enzyme may alternatively be an enzyme used in research and development applications. For example, luciferases may be used for real-time imaging of gene expression in cell cultures, individual cells and whole organisms. Further, luciferases may be used as reporter proteins in molecular studies, for example to test the activity of transcription from specific promoters in cells transfected with luciferase. Enzymes may also be used in drug design for example in the testing of enzyme inhibitors in the laboratory. Further, enzymes may be used in biosensors (for example, a blood glucose biosensor using glucose oxidase).

The luciferase enzyme may be a firefly, beetle or railroad worm luciferase, or a derivative thereof. In particular, the luciferase may be derived from a North American firefly (Phorinus pyralis), Luciola cruciata (Japanese firefly), Luciola lateralis (Japanese firefly), Luciola mingelica (russian firefly), Beneckea hanegi (marine bacterial luciferase), Pyrophorus plagiophthalamus (click beetle), Pyrocelia miyako (firefly) Ragophthalamus ohbai (railroad worm), Pyrearinus termitilluminans (click beetle), Phrixothrix hirtus (railroad worm), Phrixothrix vivianii, Hotaria parvula and Photuris pensilvanica, and mutated variants thereof.

Typically the α-galactosidase or β-galactosidase is derived from bacteria (such as Escherichia coli.), a mammal (such as human, mouse, rat) or other eukaryote.

The enzyme maybe a naturally-occurring enzyme or a synthetic enzyme. Such enzymes may be derived from a host animal, plant or a microorganism.

Microbial strains used in the production of enzymes may be native strains or mutant strains that are derived from native strains by serial culture and selection, or mutagenesis and selection using recombinant DNA techniques. For example the microorganism may be a fungus e.g. Thermomyces acermonium, Aspergillus, Penicillium, Mucor, Neurospora and Trichoderma. Yeasts such as Saccharomyces cereviseae or Pishia pastoris may also be used in the production of enzymes for use in the methods of the present invention.

A synthetic enzyme may be derived using protein-engineering techniques well known in the art such as rational design, directed evolution and DNA shuffling.

Host organisms may be transformed with a nucleotide sequence encoding a desired enzyme and cultured under conditions conducive to the production of the enzyme and which facilitate recovery of the enzyme from the cells and/or culture medium.

### Vaccine Immunogens

A vaccine immunogen suitable for use in the invention includes any immunogenic component of a vaccine. The vaccine immunogen comprises an antigen that can elicit an immune response in an individual when used as a vaccine against a particular disease or medical condition. The vaccine immunogen may be provided by itself prior to formulation of a vaccine preparation or it may be provided as part of a vaccine preparation. The vaccine immunogen may be a subunit vaccine, a conjugate useful as a vaccine or a toxoid. The vaccine immunogen may be a protein, bacterial-specific protein, mucoprotein, glycoprotein, peptide, lipoprotein, polysaccharide, peptidoglycan, nucleoprotein or fusion protein.

The vaccine immunogen may be derived from a microorganism (such as a bacterium, virus, fungi), a protozoan, a tumour, a malignant cell, a plant, an animal, a human, or an allergen. The vaccine immunogen is preferably not a viral particle. Thus, the vaccine immunogen is preferably not a whole virus or virion, virus-like particle (VLP) or virus nucleocapsid. The preservation of such viral particles is described in WO 2008/114021.

The vaccine immunogen may be synthetic, for example as derived using recombinant DNA techniques. The immunogen may be a disease-related antigen such as a pathogen-related antigen, tumour-related antigen, allergy-related antigen, neural defect-related antigen, cardiovascular disease antigen, rheumatoid arthritis-related antigen.

In particular, the pathogen from which the vaccine immunogen is derived may include human papilloma viruses (HPV), HIV, HSV2/HSV1, influenza virus (types A, B and C), para influenza virus, polio virus, RSV virus, rhinoviruses, rotaviruses, hepaptitis A virus, norwalk virus, enteroviruses, astrovinises, measles virus, mumps virus, varicella-zoster virus, cytomegalovirus, epstein-barr virus, adenoviruses, rubella virus, human T-cell lymphoma type I virus (HTLV-I), hepatitis B virus (HBV), hepatitis C virus (HCV), hepatitis D virus, poxvirus, vaccinia virus, Salmonella, Neisseria, Borrelia, Clamydia, Bordetella such as Bordetella pertussis, Plasmodium, Coxoplasma, Pneumococcus, Meningococcus, Cryptococcus, Streptococcus, Vibriocholerae, Yersinia and in particular Yersinia pestis, Staphylococcus Haemophilus, Diptheria, Tetanus, Pertussis, Escherichia, Candida, Aspergillus, Entamoeba, Giardia and Trypanasoma. The vaccine may further be used to provide a suitable immune response against numerous veterinary diseases, such as foot and mouth disease (including serotypes O, A, C, SAT-1, SAT-2, SAT-3 and Asia-1), coronavirus, bluetongue, feline leukaemia virus, avian influenza, hendra and nipah virus, pestivirus, canine parvovirus and, bovine viral diarrhoea virus.

Tumor-associated antigens include for example, melanoma-associated antigens, mammary cancer-associated antigens, colorectal cancer-associated antigens or prostate cancer-associated antigens

An allergen-related antigen includes any allergen antigen suitable for use in a vaccine to suppress an allergic reaction in an individual to which the vaccine is administered (e.g. antigens derived from pollen, dust mites, insects, food allergens, dust, poisons, parasites).

### Subunit Vaccine Immunogens

A suitable subunit vaccine immunogen includes any immunogenic subunit of a protein, lipoprotein or glycoprotein derived from a microorganism (for example a virus or bacteria). Alternatively, the subunit vaccine immunogen may be derived from a disease-related antigen such as a tumour related protein. The subunit vaccine immunogen may be a naturally occurring molecule or a synthetic protein subunit. The vaccine immunogen may be a full-length viral or bacterial protein, glycoprotein or lipoprotein or a fragment of the full-length viral or bacterial protein, glycoprotein or lipoprotein.

A viral protein suitable as a subunit vaccine immunogen may be derived from a structural or non-structural viral protein. A suitable viral subunit immunogen is capable of stimulating a subject's immune system even in the absence of other parts of the virus. A suitable viral subunit vaccine immunogen includes a capsid protein, surface glycoprotein, envelope protein, hexon protein, fiber protein, coat protein or immunogenic fragment or derivative of such proteins or glycoproteins.

For example, the viral subunit vaccine immunogen may consist of a surface protein of the Influenza A, B or C virus. In particular, the vaccine immunogen may be a hemagglutinin (HA), neuraminidase (NA), nucleoprotein, M1, M2, NS1, NS2(NEP), PA, PB1, PB1-F2 and or PB2 protein, or an immunogenic derivative or fragment of any of these proteins. The immunogen may be HAL HA2, HA3, HA4, HA5, HA6, HA7, HA8, HA9, HA10, HA11, HA12, HA13, HA14, HA15 and/or HA16, any immunogenic fragment or derivative thereof and any combination of the HA proteins, fragments or derivatives. The neuraminidase may be neuraminidase 1 (N1) or neuraminidase 2 (N2).

The viral subunit vaccine immunogen may be a hepatitis B virus viral envelope protein or a fragment or derivative thereof. For example, the subunit vaccine immunogen may be the hepatitis B surface antigen (HbsAg) or an immunogenic fragment or derivative thereof.

Typically, the bacterial subunit vaccine immunogen is a bacterial cell wall protein (e.g. flagellin, outer membrane protein, outer surface protein), a polysaccharide antigen (e.g. from Neisseria meningitis, Streptococcus pneumonia), toxin or an immunogenic fragment or derivative of such proteins, polysaccharides or toxins.

Derivatives of naturally occurring proteins include proteins with the addition, substitution and/or deletion of one or more amino acids. Such amino acid modifications can be generated using techniques known in the art, such as site-directed mutagenesis.

The subunit vaccine immunogen may be a fusion protein comprising a fusion protein partner linked with for example, a bacterial or viral protein or an immunogenic fragment or derivative thereof. A suitable fusion protein partner may prevent the assembly of viral fusion proteins into multimeric forms after expression of the fusion protein. For example, the fusion protein partner may prevent the formation of virus-like structures that might spontaneously form if the viral protein was recombinantly expressed in the absence of the fusion protein partner. A suitable fusion partner may also facilitate purification of the fusion protein, or enhance the recombinant expression of the fusion protein product. The fusion protein may be maltose binding protein, poly-histidine segment capable of binding metal ions, antigens to which antibodies bind, S-Tag, glutathione-S-transferase, thioredoxin, beta-galactosidase, epitope tags, green fluorescent protein, streptavidin or dihydrofolate reductase.

A subunit vaccine immunogen may be prepared using techniques known in the art for the preparation of for example, isolated peptides, proteins, lipoproteins, or glycoproteins. For example, a gene encoding a recombinant protein of interest can be identified and isolated from a pathogen and expressed in E. coli or some other suitable host for mass production of proteins. The protein of interest is then isolated and purified from the host cell (for example by purification using affinity chromatography).

In the case of viral subunit immunogens, the subunit may be purified from the viral particle after isolating the viral particle, or by recombinant DNA cloning and expression of the viral subunit protein in a suitable host cell. A suitable host cell for preparing viral particles must be capable of being infected with the virus and of producing the desired viral antigens. Such host cells may include microorganisms, cultured animal cells, trangenic plants or insect larvae. Some proteins of interest may be secreted as a soluble protein from the host cell. In the case of viral envelope or surface proteins, such proteins may need to be solubilized with a detergent to extract them from the viral envelope, followed by phase separation in order to remove the detergent.

A subunit vaccine immunogen may be combined in the same preparation and preserved together with one, two three or more other subunit vaccine immunogens.

### Toxoids

The invention can be applied to toxoids. A toxoid is a toxin, for example derived from a pathogen, animal or plant, that is immunogenic but has been inactivated (for example by genetic mutation, chemical treatment or by conjugation to another moiety) to eliminate toxicity to the target subject. The toxin may be for example, a protein, lipoprotein, polysaccharide, lipopolysaccharide or glycoprotein. The toxoid may thus be an endotoxin or an exotoxin that has been toxoided.

The toxoid may be a toxoid derived from a bacterial toxin such as tetanus toxin, diphtheria toxin, pertussis toxin, botulinum toxin, C. difficile toxin, Cholera toxin, shiga toxin, anthrax toxin, bacterial cytolysins or pneumolysin and fragments or derivatives thereof. The toxoid may therefore be tetanus toxoid, diphtheria toxoid or pertussis toxoid. Other toxins from which a toxoid can be derived include poisons isolated from animals or plants, for example from Crotalis atrox. Typically, the toxoid is derived from botulinum toxin or anthrax toxin. For example, the botulinum toxin may be derived from Clostridium botulinum of serotype A, B, C, D, E, F or G. The vaccine immunogen derived from a botulinum toxin may be combined in the same preparation and preserved together with one or more other vaccine immunogens derived from a botulinum toxin (eg a combination of immunogens derived from botulinum serotypes A, B, C, D, E, F or G, such as for example A, B and E).

The anthrax toxin may be derived from a strain of Bacillus anthracis. The toxoid may consist of one of more components of the anthrax toxin, or derivatives of such components, such as protective antigen (PA), the edema factor (EF) and the lethal factor (LF). Typically the toxoid derived from the anthrax toxin consists of protective antigen (PA).

The toxoid may be conjugated to another moiety, for example as a fusion protein, for use as a toxoid vaccine. A suitable moiety in a conjugate toxoid includes a substance that aids purification of the toxoid (e.g hisitidine tag) or reduces toxicity to a target subject. Alternatively, the toxoid may act as an adjuvant by increasing the immunogenicity of an antigen to which it is attached. For example, the B polysaccharide of Haemophilus influenzae may be combined with diptheria toxoid.

A vaccine immunogen may be combined in the same preparation and preserved together with one, two three or more vaccine immunogens. For example, a diphtheria toxoid may be preserved with tetanus toxoid and pertussis vaccine (DPT). Diptheria toxoid may be preserved with just tetanus toxoid (DT), or diphtheria toxoid may be preserved with diphtheria toxoid, tetanus toxoid and acellular Pertussis (DTaP).

Techniques for the preparation of toxoids are well known to those skilled in the art. Toxin genes may be cloned and expressed in a suitable host cell. The toxin product is then purified and may be converted to toxoid chemically, for example using formalin or glutaraldehyde. Alternatively, a toxin gene may be engineered so that it encodes a toxin having reduced or no toxicity e.g. by addition, deletion and/or substitution of one or more amino acids. The modified toxin can then be expressed in a suitable host cell and isolated. The toxicity of toxin genes may also be inactivated by conjugation of toxin genes or fragments thereof to a further moiety (e.g. polysaccharide or polypeptide).

### Conjugate Vaccine Immunogens

A conjugate vaccine immunogen may be a conjugate of an antigen (for example a polysaccharide or other hapten) to a carrier moiety (for example a peptide, polypeptide, lipoprotein, glycoprotein, mucoprotein or any immunostimulatory derivative or fragment thereof) that stimulates the immunogenicity of the antigen to which it is attached. For example, the conjugate vaccine immunogen may be a recombinant protein, recombinant lipoprotein or recombinant glycoprotein conjugated to an immunogen of interest (for example a polysaccharide).

The conjugate vaccine immunogen may be used in a vaccine against Streptococcus pneumonia, Haemophilus influenza, meningococcus (strains A, B, C, X, Y and W135) or pneumococcal strains. For example, the vaccine may be for example, the heptavalent Pneumococcal CRM₁₉₇ Conjugate Vaccine (PCV7), an MCV-4 or Haemophilus influenzae type b (Hib) vaccine.

A conjugate vaccine immunogen may be combined in the same preparation and preserved together with one, two three or more other conjugate vaccine immunogens.

Methods for the preparation of conjugate polysaccharide-protein conjugates are well known in the art. For example, conjugation may occur via a linker (e.g. B-propionamido, nitrophenyl-ethylamine, haloalkyl halides, glycosidic linkages).

### Preservation Mixture

The preservation mixture of the present invention comprises an aqueous solution of one or more sugars and a polyethyleneimine (PEI). The aqueous solution may be buffered. The solution may be a HEPES solution, phosphate-buffered saline (PBS) or pure water.

Sugars suitable for use in the present invention include reducing sugars such as glucose, fructose, glyceraldehydes, lactose, arabinose and maltose; and non-reducing sugars such as sucrose. The sugar may be a monosaccharide, disaccharide, trisaccharide, or other oligosaccharides. The term “sugar” includes sugar alcohols.

Monosaccharides such as galactose and mannose; dissaccharides such as lactose and maltose; trisaccharides such as raffinose and tetrasaccharides such as stachyose are envisaged. Trehalose, umbelliferose, verbascose, isomaltose, cellobiose, maltulose, turanose, melezitose and melibiose are also suitable for use in the present invention. A suitable sugar alcohol is mannitol.

Preferably, the aqueous solution is a solution of one, two or three sugars selected from sucrose, raffinose and stachyose. In particular, sucrose is a disaccharide of glucose and fructose; raffinose is a trisaccharide composed of galactose, fructose and glucose; and stachyose is a tetrasaccharide consisting of two Dα-galactose units, one Dα-glucose unit and one Dβ-fructose unit sequentially linked. A combination of sucrose and stachyose and especially sucrose and raffinose is preferred.

Preservation of biological activity is particularly effective when at least two sugars are used in the preservation mixture of the present invention. Therefore, the solution of one or more sugars comprises a solution of at least 2, at least 3, at least 4 or at least 5 sugars. Combinations of 2, 3, 4, 5, 6, 7, 8, 9, 10, etc sugars are envisaged. Preferably, the solution of two or more sugars comprises sucrose and raffinose, or sucrose and stachyose.

PEI is an aliphatic polyamine characterised by the repeating chemical units denoted as —(CH₂—CH₂—NH)—. Reference to PEI herein includes a polyethyleneimine homopolymer or copolymer. The polyethyleneimine copolymer may be a random or block copolymer. For example, PEI may consist of a copolymer of polyethyleneimine and another polymer such as polyethylene glycol (PEG). The polyethyleneimine may be linear or branched.

Reference to PEI also includes derivatised forms of a polyethyleneimine. A polyethyleneimine contains nitrogen atoms at various positions. Nitrogen atoms are present in terminal amino groups, e.g. R—NH₂, and in internal groups such as groups interrupting an alkyl or alkylene group within the polymer structure, e.g. R—N(H)—R′, and at the intersection of a polymer branch, e.g. R—N(—R′)—R″ wherein R, R′ and R″ may be alkylene groups for example. Alkyl or aryl groups may be linked to the nitrogen centres in addition to or instead of hydrogen atoms. Such alkyl and aryl groups may be substituted or unsubstituted. An alkyl group would be typically a C₁-C₄ alkyl group, e.g. methyl, ethyl, propyl, isopropyl, butyl, sec.butyl or tert.butyl. The aryl group is typically phenyl.

The PEI may be a polyethyleneimine that has been covalently linked to a variety of other polymers such as polyethylene glycol. Other modified versions of PEI have been generated and some are available commercially: branched PEI 25 kDa, jetPEI®, LMW-PEI 5.4 kDa, Pseudodendrimeric PEI, PEI-SS-PEI, PEI-SS-PEG, PEI-g-PEG, PEG-co-PEI, PEG-g-PEI, PEI-co-L lactamide-co-succinamide, PEI-co-N-(2-hydroxyethyl-ethylene imine), PEI-co-N-(2-hydroxypropyl)methacrylamide, PEI-g-PCL-block-PEG, PEI-SS-PHMPA, PEI-g-dextran 10 000 and PEI-g-transferrin-PEG, Pluronic85®/Pluronic123®-g-PEI. The PEI may be permethylated polyethyleneimine or polyethyleneimine-ethanesulfonic acid.

PEI is available in a broad range of number-average molar masses (Mₐ) for example between 300 Da and 800 kDa. Preferably, the number-average molar mass is between 300 and 2000 Da, between 500 and 1500 Da, between 1000 and 1500 Da, between 10 and 100 kDa, between 20 and 100 kDa, between 30 and 100 kDa, between 40 and 100 kDa, between 50 and 100 kDa, between 60 and 100 kDa, between 50 and 70 kDa or between 55 and 65 kDa. A relatively high Mₙ PEI of approximately 60 kDa or a relatively low Mₙ of 1200 Da is suitable.

Preferably, the weight-average molar mass (M_{w}) of PEI is between 500 Da and 1000 kDa. Most preferably, the M_{w} of PEI is between 500 Da and 2000 Da, between 1000 Da and 1500 Da, or between 1 and 1000 kDa, between 100 and 1000 kDa, between 250 and 1000 kDa, between 500 and 1000 kDa, between 600 and 1000 kDa, between 750 and 1000 kDa, between 600 and 800 kDa, between 700 and 800 kDa. A relatively high M_{w} of approximately 750 kDa or a relatively low M_{w} of approximately 1300 Da is suitable.

The weight-average molar mass (MO and number-average molar mass (Mₙ) of PEI can be determined by methods well known to those skilled in the art. For example, M_{w} may be determined by light scattering, small angle neutron scattering (SANS), X-ray scattering or sedimentation velocity. Mₙ may be determined for example by gel permeation chromatography, viscometry (Mark-Houwink equation) and colligative methods such as vapour pressure osometry or end-group titration.

Various forms of PEI are available commercially (e.g. Sigma, Aldrich). For example, a branched, relatively high molecular weight form of PEI used herein with an Mₙ of approximately 60 kDa and a M_{w} of approximately 750 kDa is available commercially (Sigma P3143). This PEI can be represented by the following formula:

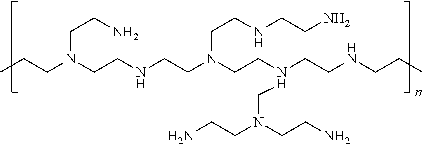

A relatively low molecular weight form of PEI used herein is also available commercially (e.g. Aldrich 482595) which has a M_{w} of 1300 Da and Mₙ of 1200 Da.

In the present invention, a preservation mixture comprising an aqueous solution of PEI and one, two or more sugars is provided. Typically, the active agent is admixed with the preservation mixture to provide the aqueous solution for drying. The concentrations of PEI and sugar that are employed for a particular active agent will depend upon the active agent. The concentrations can be determined by routine experimentation. Optimised PEI and sugar concentrations which result in the best stability can thus be selected. The PEI and sugar can act synergistically to improve stability.

The concentration of sugar in the aqueous solution for drying is greater than 0.1M. Preferably, the concentration of the sugar in the aqueous solution for drying or, if more than one sugar is present, the total concentration of sugar in the aqueous solution for drying, is at least 0.2M, 0.3M, 0.4M, 0.5M, 0.6M, 0.75M, 0.9M, 1M or 2M up to saturation e.g. saturation at room temperature or up to 3M, 2.5M or 2M. The sugar concentration or the total concentration if more than one sugar is present may be from 0.5 to 2M. When more than one sugar is present, each sugar may be present at a concentration of from 0.2M, 0.3M, 0.4M, 0.5M, 0.6M, 0.75M, 0.9M, 1M or 2M up to saturation e.g. saturation at room temperature or up to 3M, 2.5M or 2M.

The concentration of PEI in the aqueous solution for drying is generally in the range of 20 μM or less or preferably 15 μM or less based on Mₙ. The PEI concentration may be 10 W or less based on M_{w}. Such concentrations of PEI are particularly effective at preserving biological activity.

In a preferred embodiment of the invention, the PEI is provided at a concentration based on Mₙ of less than 5 μM, less than 500 nM, less than 100 nM, less than 40 nM, less than 25 nM, less than 10 nM, less than 5 nM, less than 1 nM, less than 0.5 nM, less than 0.25 nM, less than 0.1 nM, less than 0.075 nM, less than 0.05 nM, less than 0.025 Nm or less than 0.0025 nM. Typically the PEI concentration based on Mₙ is 0.0025 nM or more, 0.025 nM or more, or 0.1 nM or more. A suitable PEI concentration range based on Mₙ is between 0.0025 nM and 5 μM, or between 0.025 and 200 nM. Further preferred concentration ranges are between 0.1 nM and 5 μM and between 0.1 nM and 200 nM.

Preferably, the PEI concentration based on M_{w} is less than 5 μM, less than 1 μM, less than 0.1 μM, less than 0.01 μM, less than 5 nM, less than 4 nM, less than 2 nM, less than 1 nM, less than 0.5 nM, less than 0.25 nM, less than 0.1 nM, less than 0.05 nM, less than 0.02 nM, less than 0.002 nM or less than 0.1 nM. Typically the PEI concentration based on M_{W} is 0.00001 nM or more, 0.001 nM or more or 0.01 nM or more. A suitable PEI concentration range based on M_{w} is between 0.00001 and 20 nM, between 0.0001 and 20 nM or between 0.0001 and 5 nM.

Typically, it is found that relatively high molecular weight PEI is effective at lower concentrations than relatively low molecular weight PEI. Thus:
Where a relatively high M_{w} PEI is used, for example in the range of 20 to 1000 kDa, a concentration of PEI of between 0.001 and 5 nM based on M_{w} is preferred. Where a relatively low M_{w} PEI is used, for example in the range of 300 Da to 10 kDa, a concentration of PEI of between 0.0001 and 10 μM is preferred.
Where a relatively high Mₙ PEI is used, for example in the range of 20 to 1000 kDa, the concentration of PEI based on Mₙ is preferably between 0.001 and 100 nM. Where a relatively low Mₙ, is used, for example in the range of 1 Da to 10 kDa, a concentration of PEI of between 0.0001 and 10 μM is used.

In an embodiment, the preservation mixture initially contacted with the active agent comprises PEI at a concentration based on Mₙ of less than 2 μM and a solution of one or more sugars at a concentration of at least 0.1M, at least 0.2M, at least 0.3M, at least 0.4M, at least 0.5M, at least 0.75M, at least 0.9M, at least 1M, or at least 2M.

When the solution of one or more sugars comprises two or more sugars, the most effective concentration of PEI will be dependent on the particular type of sugar used in the preservation mixture. For example, when one of the two or more sugars is sucrose and the other is stachyose, PEI at a concentration based on Mₙ of less than 2 μM, in particular at a concentration between 0.025 nM and 2 μM, is effective at preservation. In a preferred embodiment, the method of the invention involves admixing the active agent with an aqueous solution of (i) one or more sugars wherein one of these sugars is sucrose and the other is stachyose and (ii) PEI at a concentration based on Mₙ of less than 2 μM.

When the aqueous solution of two or more sugars comprises an aqueous solution of sucrose and raffinose, the preferred concentration of PEI is found to be less than 2 μM, or in the range between 0.0025 nM and 2 μM. Therefore in a further embodiment, the method of the invention involves admixing the active agent with an aqueous solution of (i) sucrose and raffinose and (ii) PEI at a concentration between 0.0025 nM and 2 μM. Preferably, when a relatively high molecular weight PEI is used, for example between 10 and 100 kDa based on Mₙ, the concentration of PEI based on Mₙ is between 0.1 and 100 nM.

Whilst using a combination of two sugars in the preservation mixture, the present inventors investigated the effect of different molar concentration ratios of these sugars on the preservation of the active agent. Specific molar concentration ratios of one sugar to another were particularly effective but the exact ratio depended on the types of sugar used. Therefore in one embodiment of the invention in which one of the two or more sugars comprises sucrose, the concentration of sucrose relative to the other sugar is at a ratio of molar concentrations of between 3:7 and 9:1, preferably at a ratio of at least 4:6, at least 50:50, at least 6:4, at least 7:3, at least 8:2 or at least 9:1. In the case of sucrose and stachyose, a ratio of molar concentrations of sucrose:stachyose of at least 3:7, at least 4:6, at least 50:50, at least 6:4, at least 7:3, at least 3:1, at least 8:2 or at least 9:1 demonstrated particularly effective preservation. Preferably, the solution of two or more sugars comprises a solution of sucrose and stachyose at a ratio of molar concentrations of between 50:50 and 8:2.

In a further embodiment, the preservation mixture of the present invention comprises an aqueous solution of (i) two or more sugars in which one of the sugars is sucrose and the concentration of sucrose relative to the other sugar is at a ratio of molar concentrations between 3:7 and 9:1 and (ii) PEI at a concentration of less than 100 nM or at a concentration based on Mₙ between 0.025 and 100 nM.

### Preservation

The preservation techniques of the present invention are particularly suited to preservation of an active agent against desiccation, freezing and/or thermal challenge. Preservation of an active agent is achieved by drying the active agent admixed with the preservation mixture of the present invention. On drying, an amorphous solid is formed. By “amorphous” is meant non-structured and having no observable regular or repeated organization of molecules (i.e. non-crystalline).

Typically, drying is achieved by freeze-drying, snap-freezing, vacuum drying, spray-drying or spray freeze-drying. Spray freeze-drying and especially freeze-drying are preferred. By removing the water from the material and sealing the material in a vial, the material can be easily stored, shipped and later reconstituted to its original form. The active agent can thus be stored and transported in a stable form at ambient temperature without the need for refrigeration.

The drying step is generally performed as soon as the aqueous solution has been prepared or shortly afterwards. Alternatively, the aqueous solution is typically stored prior to the drying step. The polypeptide in the aqueous solution is preserved by the PEI and one or more sugars during storage.

The aqueous solution, or bulk intermediate solution, is generally stored for up to 5 years, for example up to 4 years, 3 years, 2 years or 1 year. Preferably the solution is stored for up to 6 months, more preferably up to 3 months or up to 2 months, for example 1 day to 1 month or 1 day to 1 week. Prior to drying, the solution is typically stored in a refrigerator or in a freezer. The temperature of a refrigerator is typically 2 to 8° C., preferably 4 to 6° C., or for example about 4° C. The temperature of a freezer is typically −10 to −80° C., preferably −10 to −30° C., for example about −20° C.

The solution is typically stored in a sealed container, preferably a sealed inert plastic container, such as a bag or a bottle. The container is typically sterile. The volume of the bulk intermediate solution is typically 0.1 to 100 litres, preferably 0.5 to 100 litres, for example 0.5 to 50 litres, 1 to 20 litres or 5 to 10 litres. The container typically has a volume of 0.1 to 100 litres, preferably 0.5 to 100 litres, for example 0.5 to 50 litres, 1 to 20 litres or 5 to 10 litres.

If the stored bulk intermediate solution is to be freeze-dried, it is typically poured into a freeze-drying tray prior to the drying step.

Stable storage of the solution increases the flexibility of the manufacturing process. Thus, the solution can be easily stored, shipped and later dried.

### Freeze-Drying

Freeze-drying is a dehydration process typically used to preserve perishable material or make the material more convenient for transport. Freeze-drying represents a key step for manufacturing solid protein and vaccine pharmaceuticals. However, biological materials are subject to both freezing and drying stresses during the procedure, which are capable of unfolding or denaturing proteins. Furthermore, the rate of water vapour diffusion from the frozen biological material is very low and therefore the process is time-consuming. The preservation technique of the present invention enables biological materials to be protected against the desiccation and/or thermal stresses of the freeze-drying procedure.

There are three main stages to this technique namely freezing, primary drying and secondary drying. Freezing is typically performed using a freeze-drying machine. In this step, it is important to cool the biological material below its eutectic point, the lowest temperature at which the solid and liquid phase of the material can coexist. This ensures that sublimation rather than melting will occur in the following steps. Alternatively, amorphous materials do not have a eutectic point, but do have a critical point, below which the product must be maintained to prevent melt-back or collapse during primary and secondary drying.

During primary drying the pressure is lowered and enough heat supplied to the material for the water to sublimate. About 95% of the water in the material is sublimated at this stage. Primary drying may be slow as too much heat could degrade or alter the structure of the biological material. In order to control the pressure, a partial vacuum is applied which speeds sublimation. A cold condenser chamber and/or condenser plates provide a surface(s) for the water vapour to re-solidify on.

In the secondary drying process, water molecules adsorbed during the freezing process are sublimated. The temperature is raised higher than in the primary drying phase to break any physico-chemical interactions that have formed between the water molecules and the frozen biological material. Typically, the pressure is also lowered to encourage sublimation. After completion of the freeze-drying process, the vacuum is usually broken with an inert gas, such as nitrogen, before the material is sealed.

### Snap-Freezing

In one embodiment, drying is achieved by freezing the mixture, such as by snap freezing. The term “snap freezing” means a virtually instantaneous freezing as is achieved, for example, by immersing a product in liquid nitrogen. In some embodiments it refers to a freezing step, which takes less than 1 to 2 seconds to complete.

### Vacuum Drying

In certain embodiments, drying is carried out using vacuum desiccation at around 1300 Pa. However vacuum desiccation is not essential to the invention and in other embodiments, the preservation mixture contacted with the polypeptide is spun (i.e. rotary desiccation) or freeze-dried (as further described below). Advantageously, the method of the invention further comprises subjecting the preservation mixture containing the active agent to a vacuum. Conveniently, the vacuum is applied at a pressure of 20,000 Pa or less, preferably 10,000 Pa or less. Advantageously, the vacuum is applied for a period of at least 10 hours, preferably 16 hours or more. As known to those skilled in the art, the period of vacuum application will depend on the size of the sample, the machinery used and other parameters.

### Spray-Drying and Spray Freeze-Drying

In another embodiment, drying is achieved by spray-drying or spray freeze-drying the active agent admixed with the preservation mixture of the invention. These techniques are well known to those skilled in the art and involve a method of drying a liquid feed through a gas e.g. air, oxygen-free gas or nitrogen or, in the case of spray freeze-drying, liquid nitrogen. The liquid feed is atomized into a spray of droplets. The droplets are then dried by contact with the gas in a drying chamber or with the liquid nitrogen.

### Amorphous Solid Matrix

The admixture of an active agent and preservation mixture is dried to form an amorphous solid matrix. The admixture can be dried to various residual moisture contents to offer long term preservation at greater than refrigeration temperatures e.g. within the range from about 4° C. to about 45° C., or lower than refrigeration temperatures e.g. within the range from about 0 to −70° C. or below. The amorphous solid matrix may thus have moisture content of 5% or less, 4% or less or 2% or less by weight.

In one embodiment of the invention, the amorphous solid is obtained in a dry powder form. The amorphous solid may take the form of free-flowing particles. It is typically provided as a powder in a sealed vial, ampoule or syringe. If for inhalation the powder can be provided in a dry powder inhaler. The amorphous solid matrix can alternatively be provided as a patch.

Drying onto a Solid Support

In a further embodiment of the invention, the admixture comprising active agent is dried onto a solid support. The solid support may comprise a bead, test tube, matrix, plastic support, microtiter dish, microchip (for example, silicon, silicon-glass or gold chip), or membrane. In another embodiment, there is provided a solid support onto which an active agent preserved according to the present invention is dried or attached.

### Measuring Polypeptide Preservation

Preservation in relation to a polypeptide such as a hormone, growth factor, peptide or cytokine refers to resistance of the polypeptide to physical or chemical degradation, aggregation and/or loss of biological activity such as the ability to stimulate cell growth, cell proliferation or cell differentiation, ability to stimulate cell signalling pathways, bind hormone receptors or preserve epitopes for antibody binding, under exposure to conditions of desiccation, freezing, temperatures below 0° C., below −5° C., below −10° C., below −15° C., below −20° C. or below −25° C., freeze-drying, room temperature, temperatures above −10° C., above −5° C., above 0° C., above 5° C., above 10° C., above 15° C., above 20° C., above 25° C. or above 30° C. The preservation of a polypeptide may be measured in a number of different ways. For example the physical stability of a polypeptide may be measured using means of detecting aggregation, precipitation and/or denaturation, as determined, for example upon visual examination of turbidity or of colour and/or clarity as measured by UV light scattering or by size exclusion chromatography.

The assessment of preservation of biological activity of the polypeptide will depend on the type of biological activity being assessed. For example, the ability of a growth factor to stimulate cell proliferation can be assessed using a number of different techniques well known in the art, (such as cell culture assays that monitor cells in S-phase, or the incorporation of base analogs (e.g. bromodeoxyuridine (BrdU)) as an indication of changes in cell proliferation. Various aspects of cell proliferation, or cell differentiation may be monitored using techniques such as immunofluorescence, immunoprecipitation, immunohistochemistry.

The assessment of preservation of epitopes and formation of antibody-polypeptide complexes may be determined using an immunoassay e.g. an Enzyme-linked Immunosorbant assay (ELISA).

### Uses of the Preserved Polypeptides of the Invention

The amorphous form of the preserved polypeptide enables the polypeptide to be stored for prolonged periods of time and maximises the shelf-life of the polypeptide. The potency and efficacy of the polypeptide is maintained. The particular use to which a polypeptide preserved according to the present invention is put depends on the nature of the polypeptide. Typically, however, an aqueous solution of the polypeptide is reconstituted from the dried amorphous solid matrix incorporating the polypeptide prior to use of the polypeptide.

In the case of a therapeutic polypeptide such as a hormone, growth factor, peptide or cytokine, an aqueous solution of the polypeptide can be reconstituted by addition of for example Sterile Water for Injections or phosphate-buffered saline to a dry powder comprising the preserved polypeptide. The solution of the polypeptide can then be administered to a patient in accordance with the standard techniques. The administration can be by any appropriate mode, including parenterally, intravenously, intramuscularly, intraperitoneally, transdermally, via the pulmonary route, or also, appropriately by direct infusion with a catheter. The dosage and frequency of administration will depend on the age, sex and condition of the patient, concurrent administration of other drugs, counter indications and other parameters to be taken into account by the clinician.

Generally, a therapeutic polypeptide preserved according to the invention is utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringers dextrose, dextrose and sodium chloride and lactated Ringers. Suitable physiologically-acceptable adjuvants, if necessary to keep a polypeptide complex in suspension may be chosen from thickeners such as carboxymethylcellulose, polyinylpyrrolidine, gelatine and alginates. Intravenous vehicles include fluid and nutrient replenishers and electrolyte replenishers such as those based on Ringers dextrose. Preservative and other additives, such as antimicrobials, antioxidants, chelating agents and inert gases may also be present.

Other polypeptides preserved according to the invention can, as noted above, be used as diagnostic agents.

### Measuring Antibody or Antigen-Binding Fragment Preservation

Preservation in relation to an antibody or antigen-binding fragment refers to resistance of the antibody or antigen-binding fragment to physical or chemical degradation and/or loss of biological activity such as protein aggregation or degradation, loss of antigen-binding ability, loss of ability to neutralise targets, stimulate an immune response, stimulate effector cells or activate the complement pathway, under exposure to conditions of desiccation, freezing, temperatures below 0° C., below −5° C., below −10° C., below −15° C., below −20° C. or below −25° C., freeze-drying, room temperature, temperatures above −10° C., above −5° C., above 0° C., above 5° C., above 10° C., above 15° C., above 20° C., above 25° C. or above 30° C.

The preservation of an antibody or antigen-binding fragment thereof may be measured in a number of different ways.

For example, the physical stability of antibodies may be measured using means of detecting aggregation, precipitation and/or denaturation, as determined, for example upon visual examination of turbidity and/or clarity as measured by light scattering or by size exclusion chromatography.

Chemical stability of antibodies or antigen-binding fragments may be assessed by detecting and quantifying chemically altered forms of the antibody or fragment. For example changes in the size of the antibody or fragment may be evaluated using size exclusion chromatography, SDS-PAGE and/or matrix-assisted laser desorption ionization/time-of-flight mass spectrometry (MALDI/TOF MS). Other types of chemical alteration including charge alteration, can be evaluated using techniques known in the art, for example, by ion-exchange chromatography or isoelectric focussing.

The preservation of biological activity of the antibody or antigen-binding fragment may also be assessed by measuring the ability of the antibody or antigen-binding fragment for example, to bind antigen, raise an immune response, neutralise a target (e.g. a pathogen), stimulate effector functions (e.g. opsonization, phagocytosis, degranulation, release of cytokins or cytotoxins) or activate complement pathway. Suitable techniques for measuring such biological functions are well known in the art. For example an animal model may be used to test biological functions of an antibody or antigen-binding fragment. An antigen-binding assay such as an immunoassay, may be used for example to detect antigen-binding ability.

Determining whether the antibody binds an antigen in a sample may be performed by any method known in the art for detecting binding between two protein moieties. The binding may be determined by measurement of a characteristic in either the antibody or antigen that changes when binding occurs, such as a spectroscopic change. The ability of a preserved antibody or antigen-binding fragment to bind an antigen may be compared to a reference antibody (e.g. an antibody with the same specificity of the preserved antibody or antigen-binding fragment that has not been preserved according to the methods described herein).

Generally the method for detecting antibody-antigen binding is carried out in an aqueous solution. In particular embodiments, the antibody or antigen is immobilized on a solid support. Typically, such a support is a surface of the container in which the method is being carried out, such as the surface of a well of a microtiter plate. In other embodiments, the support may be a sheet (e.g. a nitrocellulose or nylon sheet) or a bead (e.g. Sepharose or latex).

In a preferred embodiment, the preserved antibody sample is immobilized on a solid support (such as the supports discussed above). When the support is contacted with antigen, the antibody may bind to and form a complex with the antigen. Optionally, the surface of the solid support is then washed to remove any antigen that is not bound to the antibody. The presence of the antigen bound to the solid support (through the binding with the antibody) can then be determined, indicating that the antibody is bound to the antigen. This can be done for example by contacting the solid support (which may or may not have antigen bound to it) with an agent that binds to the antigen specifically.

Typically the agent is a second antibody which is capable of binding the antigen in a specific manner whilst the antigen is bound to the first immobilised sample antibody that also binds the antigen. The secondary antibody may be labelled either directly or indirectly by a detectable label. The second antibody can be labelled indirectly by contacting with a third antibody specific for the Fc region of the second antibody, wherein the third antibody carries a detectable label.

Examples of detectable labels include enzymes, such as a peroxidose (e.g. of horseradish), phosphatase, radioactive elements, gold (or other colloid metal) or fluorescent labels. Enzyme labels may be detected using a chemiluminescence or chromogenic based system.

In a separate embodiment, the antigen is immobilised on a solid support and the preserved antibody is then contacted with the immobilised antigen. The antigen-antibody complexes may be measured using a second antibody capable of binding antigen or the immobilised antibody.

Heterogeneous immunoassays (requiring a step to remove unbound antibody or antigen) or homogenous immunoassays (not requiring this step) may be used to measure the ability of preserved antibody or antigen-binding fragments to bind antigen. In a homogenous assay, in contrast to a heterogeneous assay, the binding interaction of candidate antibody with an antigen can be analysed after all components of the assay are added without additional fluid manipulations being required. Examples include fluorescence resonance energy transfer (FRET) and Alpha Screen. Competitive or non-competitive heterogeneous immunoassays may be used. For example, in a competitive immunoassay, unlabelled preserved antibody in a test sample can be measured by its ability to compete with labelled antibody of known antigen-binding ability (a control sample e.g. an antibody sampled before desiccation, heat treatment, freeze-drying and/or storage). Both antibodies compete to bind a limited amount of antigen. The ability of unlabelled antibody to bind antigen is inversely related to the amount of label measured. If an antibody in a sample is able to inhibit the binding between a reference antibody and antigen, then this indicates that such an antibody is capable of antigen-binding.

Particular assays suitable for measuring the antigen-binding ability of the preserved antibodies of the invention include enzyme-linked immunoassays such as Enzyme-Linked ImmunoSorbent Assay (ELISA), homogenous binding assays such as fluorescence resonance energy transfer (FRET), Fluorescence Polarization Immunoassay (FPIA), Microparticle Enzyme Immunoassay (MEIA), Chemiluminescence Magnetic Immunoassay (CMIA), alpha-screen surface plasmon resonance (SPR) and other protein or cellular assays known to those skilled in the art for assaying antibody-antigen interactions.

In one embodiment, using the ELISA assay, an antigen is brought into contact with a solid support (e.g. a microtiter plate) whose surface has been coated with an antibody or antigen-binding fragment preserved according to the present invention (or a reference antibody e.g. one that has not been preserved according to the method of the invention). Optionally, the plate is then washed with buffer to remove non-specifically bound antibody. A secondary antibody that is able to bind the antigen is applied to the plate and optionally, followed by another wash. The secondary antibody can be linked directly or indirectly to a detectable label. For example, the secondary antibody may be linked to an enzyme e.g. horseradish peroxidase or alkaline phosphatase, which produces a colorimetric produce when appropriate substrates are provided.

In a separate embodiment, the solid support is coated with the antigen and the preserved antibody or antigen-binding fragment is brought into contact with the immobilised antigen. An antibody specific for the antigen as preserved antibody may be used to detect antigen-antibody complexes.

In a further embodiment, the binding interaction of the preserved antibody and a target is analysed using Surface Plasmon Resonance (SPR). SPR or Biomolecular Interaction Analysis (BIA) detects biospecific interactions in real-time without labelling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules.

Information from SPR can be used to provide an accurate and quantitative measure of the equilibrium disassociation constant (D_{D}), and kinetic parameters, including Kₒₙ and K_{off} for the binding of a biomolecule to a target.

Typically, the ability of an antibody to form antibody-antigen complexes following preservation according to the present invention and incubation of the resulting product at 37° C. for 7 days is at least 10%, at least 20%, at least 30%, at least 40%, at least 50%, at least 60%, at least 70%, at least 80% or at least 90% of the ability of the antibody to form such complexes prior to such incubation, or indeed prior to preservation according to the present invention and such incubation.

### Uses of Preserved Antibodies or Antigen-Binding Fragments Thereof

Preserved antibodies or antigen-binding fragments thereof may be employed in in vivo therapeutic and prophylactic applications, in vitro and in vivo diagnostic applications and in in vitro assay and reagent applications.

In diagnostic applications, body fluids such as blood, urine, saliva, sputum, gastric juices, other blood fluid components, urine or saliva, or body tissue, may be assayed for the presence and amount of antigen that binds to the preserved antibodies or antigen-binding fragments. The assay may be performed by a number of routine methods known in the art such as immunoassays (e.g. RIA, ELISA).

For example, a sample of bodily fluid may be added to an assay mixture containing the antibody and a marker system for detection of antigen-bound antibody. By comparing the results obtained using a test sample with those obtained using a control sample, the presence of an antigen specific to a particular disease or condition may be determined. Such methods for qualitatively or quantitatively determining the antigen associated with a particular disease or condition may be used in the diagnosis of that disease or condition.

Other techniques may be used in diagnostic applications such as Western analysis and in situ protein detection by standard immunohistochemical procedures, wherein the preserved antibody or antigen-binding fragment may be labelled as appropriate for the particular technique used. Preserved antibodies or antigen-binding fragments may also be used in affinity chromatography procedures when complexed to a chromatographic support, such as a resin.

Diagnostic applications include human clinical testing in hospitals, doctors offices and clinics, commercial reference laboratories, blood banks and the home. Non-human diagnostics applications include food testing, water testing, environmental testing, bio-defence, veterinary testing and in biosensors.

Preserved antibodies or antigen-binding fragments may also be used in research applications such as in drug development, basic research and academic research. Most commonly, antibodies are used in research applications to identify and locate intracellular and extracellular proteins. The preserved antibodies or antigen binding fragments described herein may be used in common laboratory techniques such as flow cytometry, immunoprecipitation, Western Blots, immunohistochemistry, immunofluorescence, ELISA or ELISPOT.

Preserved antibodies or antigen-binding fragments for use in diagnostic, therapeutic or research applications may be stored on a solid support. In diagnostic applications for example, a patient sample such as bodily fluid (blood, urine, saliva, sputum, gastric juices etc) may be preserved according to the methods described herein by drying an admixture comprising the patient sample and preservation mixture of the present invention onto a solid support (e.g. a microtiter plate, sheet or bead). Preserved patient samples (e.g. serum) may then be tested for the presence of antibodies in the sample using for example, immunoassays such as ELISA.

Alternatively, antibodies or antigen-binding fragments of interest may be preserved according to the methods described herein by drying an admixture comprising the antibody or antigen-binding fragment and preservation mixture of the present invention onto a solid support. Patient samples may be tested for the presence of particular antigens by contacting the patient sample with a solid support onto which the antibodies or antigen-binding fragments of interest are attached. The formation of antigen-antibody complexes can elicit a measurable signal. The presence and/or amount of antigen-antibody complexes formed may be used to indicate the presence of a disease, infection or medical condition or provide a prognosis.

For therapeutic applications, the preserved antibodies or antigen-binding fragments described herein will typically find use in preventing, suppressing or treating inflammatory states, allergic hypersensitivity, cancer, bacterial or viral infection and/or autoimmune disorders (including for example, but not limited to, Type I diabetes, multiple sclerosis, rheumatoid arthritis, systemic lupus erythematosus, Crohn's disease and myasthenia gravis).

The antibody may itself be a therapeutic agent or may target a therapeutic agent or other moiety to a particular cell type, tissue or location. In one embodiment, preserved antibodies or antigen-binding fragments of the invention are conjugated to radioisotopes, toxins, drugs (e.g. chemotherpeutic drugs), enzyme prodrugs or liposomes for the treatment of a variety of diseases or conditions.

### Measuring Enzyme Preservation

Preservation in relation to an enzyme refers to resistance of the enzyme to physical degradation and/or loss of biological activity such as protein degradation, reduced catalytic activity, loss of ability to bind substrate, reduced product production, enzyme efficiency (e.g. reduced k_{cat}/Kₘ) or rate of reaction, under exposure to conditions of desiccation, freezing, temperatures below 0° C., below −5° C., below −10° C., below −15° C., below −20° C. or below −25° C., freeze-drying, room temperature, temperatures above −10° C., above −5° C., above 0° C., above 5° C., above 10° C., above 15° C., above 20° C., above 25° C. or above 30° C. The preservation of an enzyme may be measured in a number of different ways. For example the physical stability of an enzyme may be measured using means of detecting aggregation, precipitation and/or denaturation, as determined, for example upon visual examination of turbidity or of colour and/or clarity as measured by UV light scattering or by size exclusion chromatography.

The preservation of catalytic activity of the enzyme may be assessed using an enzyme assay to measure the consumption of substrate or production of product over time. The catalytic activity of a preserved enzyme may be compared with a reference enzyme having the same specificity that has not been preserved according to the present invention.

Changes in the incoporation of radioisotopes, fluorescence or chemiluminescence of substrates, products or cofactors of an enzymatic reaction or substances bound to such substrates, products or cofactors, may be used to monitor the catalytic activity of the enzyme in such assays.

For example, a continuous enzyme assay may be used (e.g. a spectrophotometric assay, a fluorimetric assay, calorimetric assay, chemiluminescent assay or light scattering assay) or a discontinuous enzyme assay (e.g. a radiometric or chromatographic assay). In contrast to continuous assays, discontinuous assays involve sampling of the enzyme reaction at specific intervals and measuring the amount of product production or substrate consumption in these samples.

For example, spectrophotometric assays involve the measurement of changes in the absorbance of light between products and reactants. Such assays allow the rate of reaction to be measured continuously and are suitable for enzyme reactions that result in a change in the absorbance of light. The type of spectrophotometric assay will depend on the particular enzyme/substrate reaction being monitored. For example, the coenzymes NADH and NADPH absorb UV light in their reduced forms, but do not in their oxidised forms. Thus, an oxidoreductase using NADH as a substrate could therefore be assayed by following the decrease in UV absorbance as it consumes the coenzyme.

Radiometric assays involve the incorporation or release of radioactivity to measure the amount of product made over the time during an enzymatic reaction (requiring the removal and counting of samples). Examples of radioactive isotopes suitable for use in these assays include ¹⁴C, ³²P, ³⁵C and ¹²⁵I. Techniques such as mass spectrometry may be used to monitor the incorporation or release of stable isotopes as substrate is converted into product.

Chromatographic assays measure product formation by separating the reaction mixture into its components by chromatography. Suitable techniques include high-performance liquid chromatography (HPLC) and thin layer chromatography.

Fluorimetric assays use a difference in the fluorescence of substrate from product to measure the enzyme reaction. For example a reduced form may be fluorescent and an oxidised form non-fluorescent. In such an oxidation reaction, the reaction can be followed by a decrease in fluorescence. Reduction reactions can be monitored by an increase in fluorescence. Synthetic substrates can also be used that release a fluorescent dye in an enzyme catalysed reaction.

Chemiluminescent assays can be used for enzyme reactions that involve the emission of light. Such light emission can be used to detect product formation. For example an enzyme reaction involving the enzyme luciferase involves production of light from its substrate luciferin. Light emission can be detected by light sensitive apparatus such as a luminometer or modified optical microscopes.

### Uses of the Preserved Enzymes of the Invention

The amorphous form of the preserved enzyme enables the enzyme to be stored for prolonged periods of time and maximises the shelf-life of the enzyme. The potency and efficacy of the enzyme is maintained. The particular use to which an enzyme preserved according to the present invention is put depends on the nature of the enzyme. Typically, however, an aqueous solution of the enzyme is reconstituted from the dried amorphous solid matrix incorporating the enzyme prior to use of the enzyme.

In the case of a therapeutic enzyme for example, an aqueous solution of the enzyme can be reconstituted by addition of for example Water for Injections or phosphate-buffered saline to a dry powder comprising the preserved enzyme. The solution of the enzyme can then be administered to a patient in accordance with the standard techniques. The administration can be by any appropriate mode, including parenterally, intravenously, intramuscularly, intraperitoneally, transdermally, via the pulmonary route, or also, appropriately by direct infusion with a catheter. The dosage and frequency of administration will depend on the age, sex and condition of the patient, concurrent administration of other drugs, counter indications and other parameters to be taken into account by the clinician.

Generally, a therapeutic enzyme preserved according to the invention is utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringers dextrose, dextrose and sodium chloride and lactated Ringers. Suitable physiologically-acceptable adjuvants, if necessary to keep a polypeptide complex in suspension may be chosen from thickeners such as carboxymethylcellulose, polyinylpyrrolidine, gelatine and alginates. Intravenous vehicles include fluid and nutrient replenishers and electrolyte replenishers such as those based on Ringers dextrose. Preservative and other additives, such as antimicrobials, antioxidants, chelating agents and inert gases may also be present.

Other enzymes preserved according to the invention can, as noted above, be used as diagnostic agents, in biosensors, in the production of bulk products such as glucose or fructose, in food processing and food analysis, in laundry and automatic dishwashing detergents, in the textile, pulp, paper and animal feed industries, as a catalyst in the synthesis of fine chemicals, in clinical diagnosis or in research applications such as genetic engineering.

### Measuring Vaccine Immunogen Preservation

Preservation in relation to a vaccine immunogen refers to resistance of the vaccine immunogen to physical or chemical degradation and/or loss of biological activity such as protein degradation, loss of ability to stimulate a cellular or humoral immune response or loss of ability to stimulate antibody production or bind antibodies under conditions of desiccation, freezing, temperatures below 0° C., below −5° C., below −10° C., below −15° C., below −20° C. or below −25° C., freeze-drying, room temperature, temperatures above −10° C., above −5° C., above 0° C., above 5° C., above 10° C., above 15° C., above 20° C., above 25° C. or above 30° C.

The preservation of a vaccine immunogen may be measured in a number of different ways. For example, antigenicity may be assessed by measuring the ability of a vaccine immunogen to bind to immunogen-specific antibodies. This can be tested in various immunoassays known in the art, which can detect antibodies to the vaccine immunogen. Typically an immunoassay for antibodies will involve selecting and preparing the test sample, such as a sample of preserved vaccine immunogen (or a reference sample of vaccine immunogen that has not been preserved in accordance with the methods of the present invention) and then incubating with antiserum specific to the immunogen in question under conditions that allow antigen-antibody complexes to form.

Further, antibodies for influenza haemagglutinin and neuraminidase can be assayed routinely in the haemagglutanin-inhibition and neuraminidase-inhibition tests, an agglutination assay using erythrocytes, or using the single-radial diffusion assay (SRD). The SRD is based on the formation of a visible reaction between the antigen and its homologous antibody in a supporting agarose gel matrix. The virus immunogen is incorporated into the gel and homologous antibodies are allowed to diffuse radially from points of application through the fixed immunogens. Measurable opalescent zones are produced by the resulting antigen-antibody complexes.

### Uses of Preserved Vaccine Immunogens

A preserved vaccine immunogen of the present invention is used as a vaccine.

For example, a preserved subunit vaccine immunogen, conjugate vaccine immunogen or toxoid immunogen is suitable for use as a subunit, conjugate or toxoid vaccine respectively. As a vaccine the preserved vaccine immunogens of the invention may be used for the treatment or prevention of a number of conditions including but not limited to viral infection, sequelae of viral infection including but not limited to viral-, animal- or insect-induced toxicity, cancer and allergies. Such antigens contain one or more epitopes that will stimulate a host's immune system to generate a humoral and/or cellular antigen-specific response.

The preserved vaccine immunogen of the invention may be used as a vaccine in the prophylaxis or treatment of infection by viruses such as human papilloma viruses (HPV), HIV, HSV2/HSV1, influenza virus (types A, B and C), para influenza virus, polio virus, RSV virus, rhinoviruses, rotaviruses, hepaptitis A virus, norwalk virus, enteroviruses, astroviruses, measles virus, mumps virus, varicella-zoster virus, cytomegalovirus, epstein-barr virus, adenoviruses, rubella virus, human T-cell lymphoma type I virus (HTLV-I), hepatitis B virus (HBV), hepatitis C virus (HCV), hepatitis D virus, poxvirus, and vaccinia virus. The vaccine may further be used to provide a suitable immune response against numerous veterinary diseases, such as foot and mouth disease (including serotypes O, A, C, SAT-1, SAT-2, SAT-3 and Asia-1), coronavirus, bluetongue, feline leukaemia virus, avian influenza, hendra and nipah virus, pestivirus, canine parvovirus and bovine viral diarrhoea virus. Alternatively, the vaccine may be used to provide a suitable immune response against animal- or insect-induced toxicity (for example as induced by snake venom or other animal poisons). In one embodiment, the vaccine is a multivalent vaccine.

The vaccine compositions of the present invention comprise a vaccine immunogen admixed with the preservation mixture of the invention containing one or more sugars and PEI. The vaccine composition may further comprise appropriate buffers and additives such as antibiotics, adjuvants or other molecules that enhance presentation of the vaccine immunogen to specific cells of the immune system.

A variety of adjuvants well known in the art can be used in order to increase potency of the vaccine and/or modulate humoral and cellular immune responses. Suitable adjuvants include, but are not limited to, oil-in-water emulsion-containing adjuvants or water in oil adjuvants, such as mineral oil, aluminium-based adjuvants, squalene/phosphate based adjuvants, Complete/Incomplete Freunds Adjuvant, cytokines, an immune stimulating complex (ISCOM) and any other substances that act as immuno stimulating agents to enhance the effectiveness of the vaccine. The aluminium-based adjuvant includes aluminium phosphate and aluminium hydroxide. An ISCOM may comprise cholesterol, lipid and/or saponin. The ISCOM may induce a wide range of systemic immune responses.

The vaccine composition of the present invention can be in a freeze-dried (lyophilised) form in order to provide for appropriate storage and maximize the shelf-life of the preparation. This will allow for stock piling of vaccine for prolonged periods of time and help maintain immunogenicity, potency and efficacy. The preservation mixture of the present invention is particularly suited to preserve viral substances against desiccation and thermal stresses encountered during freeze-drying/lyophilisation protocols. Therefore, the preservation mixture is suitable for adding to the vaccine immunogen soon after harvesting and before subjection of the sample to the freeze-drying procedure.

To measure the preservation of a vaccine prepared in accordance with the present invention, the potency of the vaccine can be measured using techniques well known to those skilled in the art. For example, the generation of a cellular or humoral immune response can be tested in an appropriate animal model by monitoring the generation of antibodies or immune cell responses to the vaccine. The ability of vaccine samples prepared in accordance with the method of the present invention to trigger an immune response may be compared with vaccines not subjected to the same preservation technique.

The following Examples illustrate the invention.

### Example 1

### Stabilizing Calcitonin

### 1. Sample Preparation

Vials of desiccated hCT (human calcitonin) were obtained from Sigma (code T3535) and reconstituted in PBS (Sigma) to a final concentration of 3 μg/μl using the manufacturer's stated mass content before each experiment.

An aqueous solution of the sugars sucrose and raffinose (sugar mix) and PEI (Sigma catalogue number: P3143—solution 50% w/v in water; Mₙ 60,000) was prepared as 4 parts 1.82M sucrose solution: 1 part 0.75M raffinose: 1 part PEI (PEI concentration of 150 nM based on M.). A 50 μl aliquot of the excipient was added to 3 μl hCT and the volume brought up to 60 μl with PBS. The final concentrations of the sugars and PEI were:

sucrose: 1.03M

raffinose: 0.09M

PEI: 21 nM (based on Mₙ of 60,000)

For controls, PBS was used in place of excipient. Multiple 60 μl aliquots were prepared for testing as follows:
1. Calcitonin resuspended in PBS and frozen
2. Calcitonin resuspended in PBS and freeze-dried
3. Calcitonin+sugar mix freeze-dried
4. Calcitonin+sugar mix freeze-dried+heated (at 45° C. for 16 hours)
5. Calcitonin+excipient freeze-dried (invention)
6. Calcitonin+excipient freeze-dried and heat-treated (at 45° C. for 16 hours) (invention)

The 60 μl aliquots were distributed into separate glass vials (Adelphi Glass), and frozen or freeze-dried. The vials were freeze-dried in a Modulyo D freeze-dryer (Thermo-Fisher). More specifically, the vials were frozen at −80° C. in freeze-dryer trays containing 30 ml water with rubber stoppers partially in. Frozen vials were transferred to the freeze-dryer stoppering shelf of the pre-cooled freeze dryer and dried for 16 hours. Rubber stoppers were lowered fully into the vials under a vacuum before removing from freeze dryer.

Vials from both the frozen and the freeze-dried sample groups were then either stored at −20° C. or subjected to heat challenge. Desiccated samples were then reconstituted to their original volume of 60 μl using sterile ddH₂O (double distilled water). 50 μl of each solution was then used for the first dilution of each series.

### 2. ELISA Protocol

A NUNC ELISA plate (MaxiSorp™ Surface) was coated for 2 hr at room temperature (RT) with 1000 of purified rabbit anti-human calcitonin polyclonal antibody (Abcam, code ab8553) diluted 1:2000 in PBS. Wells were then washed once with PBS before being blocked with 100 μl blocking solution (5% sucrose, 5% bovine serum albumin (BSA) solution in PBS; prepared fresh) overnight at 4° C. Plates were then washed three times with PBS.

In preparation for the dilution series, 50 μl PBS was then added to each well. hCT samples at a concentration of 0.15 ug/ml, prepared as described above in “Sample preparation”, were then added as 50 μl aliquots to the first well of each dilution series, to give an initial concentration of 0.075 ug/ul, and diluted 2-fold down each series. 50 μl of solution was discarded from the last dilution point of each series such that all wells contained 50 μl. Plates were then incubated for 2 hours at room temperature and then washed 3 times with PBS.

The secondary, horse-radish peroxidase (HRP)-conjugated antibody was then added. 100 μl purified monoclonal HRP-conjugated mouse anti-hCT antibody (Abcam, code ab11484) at a dilution of 1:2000 in PBS was added to each well and incubated for 2 hr at RT. Wells were then washed once with 100 μl PBS containing 0.05% Tween 20 and then five time with PBS.

Bound active hCT was then quantified. 100 μl of freshly prepared colorimetric reagent mix, TMB (3,3′,5,5′ tetramethylbenzidine) and H₂O₂, was added to each well prior to a 30 min incubation in the dark. Plates were then read at 450 nm using an automated plate reader and the optical density (OD) values exported into Excel.

### 3. Results & Discussion

FIG. 1 summarizes the results. FIG. 1 shows the averaged result of detectable hCT (using OD at a wavelength of 450 nm) as measured by ELISA following subjecting the samples outlined above to heat challenge for an extended period. It can be clearly seen that stabilisation of freeze-dried samples is dramatically improved when the excipient of 1.03M sucrose, 0.09M raffinose and 21 nM PEI (based on Mₙ) has been applied. Interestingly, the combination of sugars and PEI substantially protects the freeze-dried sample compared to the positive control which was not subjected to freeze-drying or heat challenge, but instead subjected to a second freeze.

### Example 2

### Preservation of Human Recombinant G-CSF

### 1. Materials and Methods

### Materials

An antibody for phospho-specific ERK1/2 was purchased from Sigma (Dorset, UK) and anti-ERK 2 was obtained from (Zymed UK). PEI (Mₙ 60,000; Sigma catalogue number: P3143), sucrose (Sigma), raffinose (Fluka), PBS (Sigma), glass vials (Adelphi glass), rubber stoppers (Adelphi glass) and G-CSF (Sigma).

### Sample Preparation

A lyophilised sample of G-CSF was reconstituted to a concentration of 10 μg/ml. 160 μl of sucrose (1.82M) and 40 μl of raffinose (0.75M) were mixed with 50 μl of PEI (at a concentration of 150 nM based on Mₙ) to complete the preservation mixture. 50 μl of the reconstituted G-CSF solution was added and mixed well. The final concentrations of the sugars and PEI were:

sucrose: 0.91M

raffinose: 0.125M

PEI: 25 nM (based on Mₙ)

100 μl aliquots of the final mixture was distributed into separate vials, and frozen or freeze-dried. Lyophilisation was carried out overnight as described in Example 1. Samples from both the frozen and the freeze-dried groups were then either stored at −20° C. or heated at 37° C. for 72 hours. Following incubation, the samples were reconstituted in RPMI prior to use.

### Tissue Culture

HL60 cells (shown to be mycoplasma free) were maintained in phenol red containing RPMI 1640 supplemented with 10% foetal bovine serum (FBS) and 2 mM glutamine. Cells were passaged weekly and medium was replenished every 2-3 days.

### Cell Stimulation Assays

For stimulation assays HL60 cells were harvested and transferred to serum free medium at a density of 5×10⁵ per well of a 6 well plate. After 24 hours cells were stimulated for 5 minutes with the treatments shown in FIG. 2 (100 ng/ml G-CSF) and as indicated below:
FIG. 2 panel A: Control (serum starved+PBS), UT G-CSF (untreated G-CSF) and freeze thaw G-CSF (standard G-CSF mixed with excipient and frozen) samples.
FIG. 2 panel B: Control (serum starved+PBS), UT G-CSF (untreated G-CSF) and Excipient/HT G-CSF (G-CSF mixed with excipient then heated) samples.
FIG. 2 panel C: Control (serum starved+PBS), UT G-CSF (untreated G-CSF) and G-CSF Excipient/FD (G-CSF mixed with excipient and freeze dried) samples.
FIG. 2 panel D: Control (serum starved+PBS), UT G-CSF (untreated G-CSF) and G-CSF Excipient/FD/HT (G-CSF mixed with excipient, freeze dried and heat treated) samples.

Whole cell extracts were resolved by SDS-PAGE and then transferred to nylon membranes, which were immunoprobed with antibodies against phosphorylated and total ERK1/2.

### Preparation of Whole Cell Extracts for Immunoblots

Cell suspensions were harvested (1000 rpm for 5 minutes) and washed with ice-cold PBS. Cell pellets were then lysed in extraction buffer (1% (v/v) Triton X100, 10 mM Tris-HCl, pH 7.4, 5 mM EDTA, 50 mM NaCl, 50 mM sodium fluoride 2 mM Na₃ VO₄ and 1 tablet of Complete™ inhibitor mix (Boehringer) per 10 ml of buffer) and homogenised by passage through a 26-gauge needle 6 times.

The lysate was incubated on ice for 10 minutes then clarified by centrifugation (14,000 rpm for 10 minutes at 4° C.). The protein concentration was then quantified using the BSA reagent (Biorad, Inc.). Equal amounts of protein (50 mg) were resolved by SDS-PAGE (10% gels) and then subjected to immunoblot analysis. Antigen-antibody interactions were detected with ECL (Pierce, UK).

### 2. Results

The results are shown in FIG. 2. Under serum starved conditions 70-80% of cells were arrested in G0. Assessment of the level of phosphorylated ERK1/2 showed limited expression in serum starved vehicle treated control as expected. G-CSF (native) was shown to enhance phosphorylation without any effect on total ERK1/2 levels. Further:
G-CSF mixed with the preservation mixture (excipient) then showed a similar profile to the native G-CSF as indicated in FIG. 2A.
Assessment of the effect of mixing G-CSF with the excipient, followed by heat treatment indicated a marked loss of activity compared to untreated G-CSF (FIG. 2B).
The combination of G-CSF with the excipient followed by freeze-drying appeared to maintain the potency of G-CSF compared to the untreated G-CSF form (FIG. 2C).
Of particular note the excipient combined with freeze-drying appeared to protect G-CSF against heat inactivation (compare FIG. 2D with FIG. 2B).

### Example 3

### Stabilisation of Anti-TNFα Antibody

### 1. Experimental Outline

The following samples of anti-human tumor necrosis factor-α antibodies (rat monoclonal anti-TNFα, Invitrogen Catalogue No.: SKU#RHTNFA00) were prepared and their preservation assessed by the retention of their normal functional activity of binding hTNFα using an ELISA assay after the indicated treatment:
1. anti-hTNFα rat mAb (test)—no treatment+PBS (4° C.) (control)
2. anti-hTNFα rat mAb—freeze dried+excipient and stored at 4° C.
3. anti-hTNFα rat mAb—freeze dried+excipient and heat treated at 65° C. for 24 hours
4. anti-hTNFα rat mAb—heat treated+PBS at 65° C. for 24 hours

The excipient contained a final concentration of 0.91M sucrose, 0.125M raffinose and 25 nM PEI (Mₙ 60,000). An ELISA plate (NUNC ELISA plate (MaxiSorp™)) was coated with the rat monoclonal antibody (rat hTNFα mAb) directed against hTNFα. hTNFα was added to the plate and allowed to bind to the coated plate. Bound hTNFα was detected with a biotinylated polyclonal rat anti hTNFα, which subsequently was visualized using a Streptavidin-Horseradish peroxidase (HRP) conjugate in a colorimetric reaction by adding 100 μl TMB substrate (3,3′,5,5′-tetramethylbenzidine and hydrogen peroxide).

After an incubation period of 30 minutes in the dark, the reaction was stopped by adding 50 μl 1N of hydrochloric acid. ELISA plates were subsequently read using an ELISA reader (Synergy HT) at 450 nm. Results were plotted into Excel.

### 2. Method

### Materials

NUNC ELISA plate (MaxiSorp™). Anti-hTNFα rat mAb (Catalogue No.: SKU#RHTNFA00, Invitrogen, 200 μg/ml). Anti-hTNFα detection kit (TiterZyme® EIA, assay designs, Cat. No.: 900-099)

### Excipient Preparation

An excipient was prepared by mixing 160 μl of sucrose (1.82M), 40 μl of raffinose (0.75M) and 50 μl of PEI (at a concentration of 150 nM as estimated using a Mₙ of 60,000).

### Preparation of Samples for Freeze-Drying (FD)

The following samples were prepared and tested after the indicated period of time, in the ELISA assay.
1. anti-hTNFα rat mAb (test)—no treatment+PBS (4° C.) (control)
2. anti-hTNFα rat mAb—freeze dried+excipient and stored at 4° C.
3. anti-hTNFα rat mAb—freeze dried+excipient and heat treated at 65° C. for 24 hours
4. anti-hTNFα rat mAb—heat treated+PBS at 65° C. for 24 hours

50 μl of undiluted anti-TNFα antibody (rat mAb) was added to 250 μl of the above excipient preparation. The final concentration of each component in the excipient mix was 0.91M sucrose, 0.125M raffinose and 25 nM PEI (based on Mₙ of 60,000). 100 μl aliquots were added into freeze-drying vials and subjected onto a VirTis Freeze-dryer.

After freeze-drying of samples, vials were stored at 4° C. or heat treated for varying lengths of time and reconstituted in PBS (333 μl per 100 μl FD aliquot) prior to the assay.

50 μl of control (sample 1 above) rat mAb (1:20 dilution in PBS) and 50 μl of each reconstituted solution were coated onto an ELISA plate overnight at 4° C. The rest of the assay was performed according to manufacturers' outline (TiterZyme® EIA, assay designs, Cat. No.: 900-099).

### Set up of ELISA

An ELISA plate was coated with 50 μl (1:20 dilution) of purified anti-hTNFα rat mAb and incubated overnight (o/n) at 4° C.

A human TNFα standard was prepared according to manufacturers' outline (starting concentration at 1000 pg/ml) and distributed in duplicate onto the plate.

A rabbit polyclonal antibody to hTNFα, streptavidin conjugated to horseradish peroxidase, TMB substrate and stop solution were distributed according to the commercial kit (TiterZyme® EIA, see above) outline. Briefly, after each incubation step, four washes were performed before the addition of the next reagent and incubation for a further 60 min at 37° C. After adding the stop solution, plates were read at 450 nm. Blank wells (coated with the rat mAb against hTNFα, but no addition of recombinant hTNFα) were run in parallel.

As a positive control, a pre-coated ELISA strip from the kit was run in parallel to verify that all used reagents from commercial kit were functional (data not shown).

### 3. Results

Following the treatments outlined above, the ELISA enabled us to assess the level of remaining antibody activity. The results are shown in FIG. 3.

It was clear the inclusion of the excipient preparation prior to freeze drying of the antibody enabled the said antibody to withstand to a significantly higher level, heat challenge for significantly longer periods. Antibody diluted in PBS and subjected to heat challenge lost greater than 40% of its efficacy over the same time period.

### Example 4

### Preservation of Luciferase

All solutions were prepared in 5 ml glass vials (Adelphi Glass). 180 μl of sucrose (1.82M, Sigma) and 200 of stachyose (0.75M, Sigma) were added giving a total 200 μl volume for the sugar mix. 50 μl of PEI (Sigma catalogue number P3143, Mₙ 60,000) was then added at various concentrations to complete the preservation mixture. Finally, 50 μl of luciferase (Promega) at 0.1 mg/ml or 500 of phosphate-buffered saline (PBS, Sigma) was added and the mixture vortexed. The final concentrations of PEI and sugars were:

PEI: 27 nM, 2.7 nM or 0.27 nM

sucrose: 1.092 M, and

stachyose: 0.0499M.

A control containing 3000 of PBS was also set up. All vials were set up in triplicate.

The vials were freeze-dried in a Modulyo D freeze-dryer (ThermoFisher). More specifically, the vials were frozen at −80° C. in freeze-dryer trays containing 30 ml water with rubber stoppers partially in. Frozen vials were transferred to the freeze-dryer stoppering shelf of the pre-cooled freeze dryer and dried for 16 hours. Rubber stoppers were lowered fully into the vials under a vacuum before removing from freeze dryer.

The vials contained a free-flowing freeze-dried powder. The powder was reconstituted by adding 1 ml PBS. 100 μl of each resulting solution was transferred to a 96 well plate. Luciferase assay reagent was added to each well according to manufacturer's instructions and luminescence was read on a Synergy 2 luminometer.

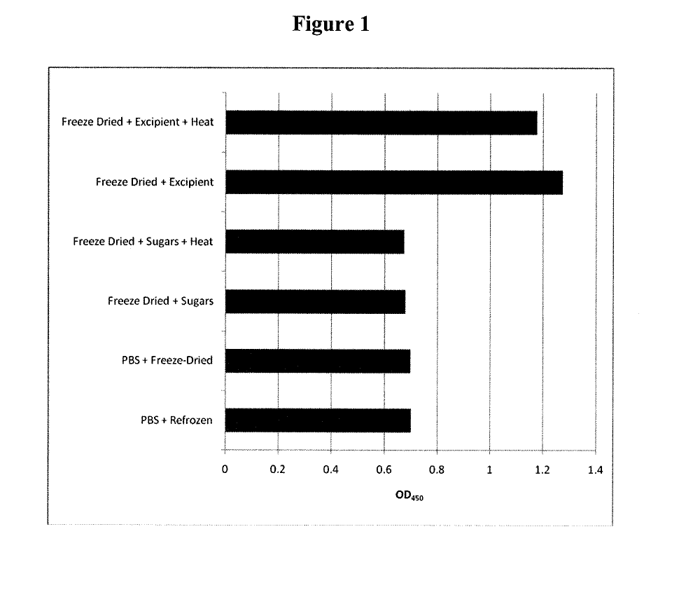

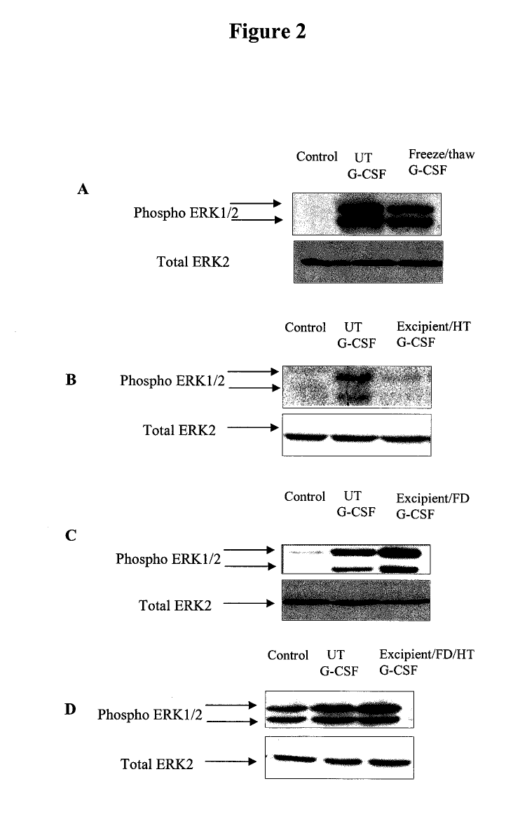

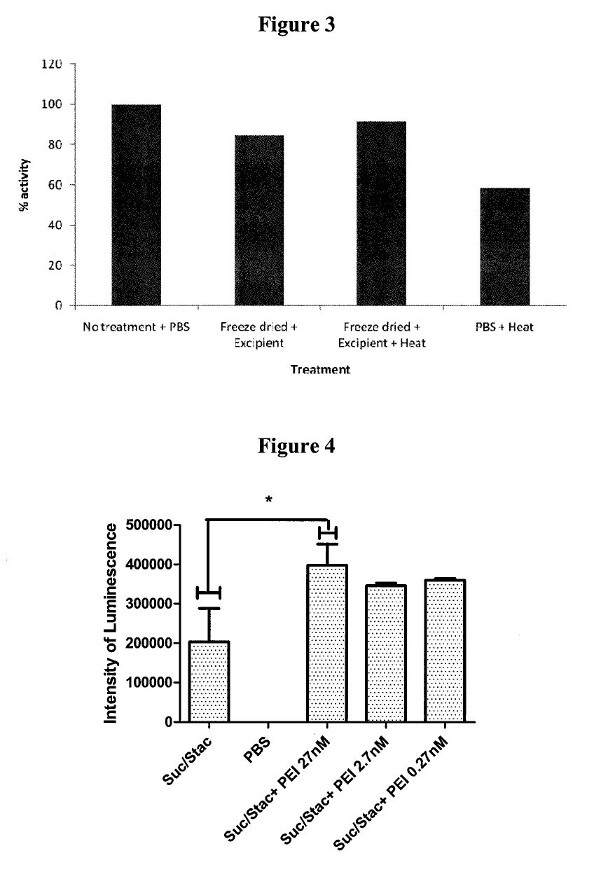

The results are shown in FIG. 4. A students T test was performed to analyse significance between different excipients using PRISM Graphpad software version 4.00. The P value summaries are *p<0.10; **p<0.05; ***p<0.005.

### Example 5

### Preservation of β-Galactosidase

All solutions were prepared in 5 ml glass vials (Adelphi Glass). 1600 of sucrose (1.82M, Sigma) and 40 μl of raffinose (1M, Sigma) were added giving a total 200 μl volume for the sugar mix. 50 μl of PEI (Sigma catalogue number P3143, Mₙ 60,000) was then added at various concentrations to complete the preservation mixture. Finally, 50 μl of β-galactosidase (100 units per ml, Sigma) or 50 μl of phosphate-buffered saline (PBS, Sigma) was added and the mixture vortexed. The final concentrations of PEI and sugars were:

PEI: 13 μM, 2.6 μM, 0.26 μM, 26 nM or 2.6 nM

sucrose: 0.97 M, and

raffinose: 0.13M.

To evaluate the effect of PEI without sugars, 50 μl of PEI was added to 250 μl of PBS. A control containing 300 μl of PBS was also set up. All vials were set up in triplicate.

The vials were freeze-dried in a Modulyo D freeze-dryer (ThermoFisher). More specifically, the vials were frozen at −80° C. in freeze-dryer trays containing 30 ml water with rubber stoppers partially in. Frozen vials were transferred to the freeze-dryer stoppering shelf of the pre-cooled freeze dryer and dried for 16 hours. Rubber stoppers were lowered fully into the vials under a vacuum before removing from freeze dryer.

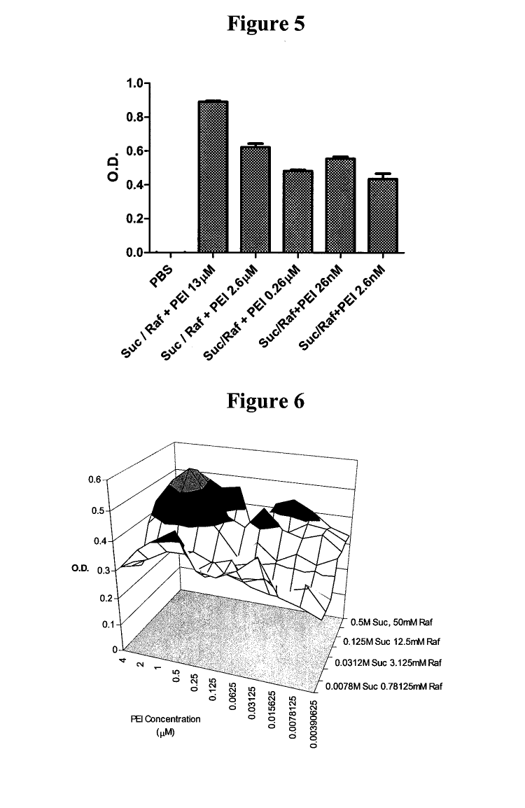

The vials contained a free-flowing freeze-dried powder. The powder was reconstituted by adding 1 ml PBS. 100 μl of each resulting solution was transferred to a 96 well plate. β-galactosidase activity was assayed with x-gal as the substrate. The results are shown in FIG. 5. A students T test was performed to analyse significance between different excipients using PRISM Graphpad software version 4.00. The P value summaries are *p<0.10; **p<0.05; ***p<0.005.

### Example 6

### Stabilisation of Anti-TNFα Antibody

### 1. Materials

L929 cells (ECCAC 85011426)

### PEI (Sigma P3143, Lot 127K0110, Mn 60,000)

### Sucrose (Suc, Sigma 16104, Lot 70040)

### Raffinose (Raf, Sigma R0250, Lot 039K0016)

Phosphate buffered saline (PBS, Sigma D8662, Lot 118K2339)

### Water (Sigma W3500, Lot 8M0411)

### Thiazolyl Blue Tetrazolium Bromide (MTT)

Anti-human TNFα purified antibody (Invitrogen RHTNFAOO, Lots 555790A and 477758B). Stock solution of 200 μg per ml PBS prepared and stored at 2-8° C.
5 ml glass vials (Adelphi Tubes VCD005)
14 mm freeze-drying stoppers (Adelphi Tubes FDIA14WG/B)
14 mm caps (Adelphi Tubes CWPP14)
Total recovery HPLC vials (Waters 18600384 C, Lot 0384691830)

### 2. Method

### Preparation of Samples

Excipients were prepared in PBS in accordance with the components listed in Table 1. PEI concentrations are based on Mn. 250 μl of each excipient mixture and 10 μg of the anti-TNFα antibody in 50 μl PBS were then placed in appropriately labelled 5 ml glass vials and vortexed. After vortexing, vials were transferred to the stoppering shelf of a VirTis Advantage freeze-dryer (Biopharma Process Systems). The final concentrations of sucrose, raffinose and PEI in the vials prior to freeze-drying are shown in Table 1.

TABLE 1

| | | | | | |
|---|---|---|---|---|---|
| 0.5M Suc, | 0.5M Suc, | 0.5M Suc, | 0.5M Suc, | 0.5M Suc, | 0.5M Suc, |
| 50 mM Raf | 50 mM Raf | 50 mM Raf | 50 mM Raf | 50 mM Raf | 50 mM Raf |
| 4 μM PEI | 2 μM PEI | 1 μM PEI | 0.5 μM PEI | 0.25 μM PEI | 0.125 μM PEI |
| 0.25M Suc | 0.25M Suc | 0.25M Suc | 0.25M Suc | 0.25M Suc | 0.25M Suc |
| 25 mM Raf | 25 mM Raf | 25 mM Raf | 25 mM Raf | 25 mM Raf | 25 mM Raf |
| 4 μM PEI | 2 μM PEI | 1 μM PEI | 0.5 μM PEI | 0.25 μM PEI | 0.125 μM PEI |
| 0.125M Suc | 0.125M Suc | 0.125M Suc | 0.125M Suc | 0.125M Suc | 0.125M Suc |
| 12.5 mM Raf | 12.5 mM Raf | 12.5 mM Raf | 12.5 mM Raf | 12.5 mM Raf | 12.5 mM Raf |
| 4 μM PEI | 2 μM PEI | 1 μM PEI | 0.5 μM PEI | 0.25 μM PEI | 0.125 μM PEI |
| 0.0625M Suc | 0.0625M Suc | 0.0625M Suc | 0.0625M Suc | 0.0625M Suc | 0.0625M Suc |
| 6.25 mM Raf | 6.25 mM Raf | 6.25 mM Raf | 6.25 mM Raf | 6.25 mM Raf | 6.25 mM Raf |
| 4 μM PEI | 2 μM PEI | 1 μM PEI | 0.5 μM PEI | 0.25 μM PEI | 0.125 μM PEI |
| 0.0312M Suc | 0.0312M Suc | 0.0312M Suc | 0.0312M Suc | 0.0312M Suc | 0.0312M Suc |
| 3.125 mM Raf | 3.125 mM Raf | 3.125 mM Raf | 3.125 mM Raf | 3.125 mM Raf | 3.125 mM Raf |
| 4 μM PEI | 2 μM PEI | 1 μM PEI | 0.5 μM PEI | 0.25 μM PEI | 0.125 μM PEI |
| 0.0156M Suc | 0.0156M Suc | 0.0156M Suc | 0.0156M Suc | 0.0156M Suc | 0.0156M Suc |
| 1.56 mM Raf | 1.56 mM Raf | 1.56 mM Raf | 1.56 mM Raf | 1.56 mM Raf | 1.56 mM Raf |
| 4 μM PEI | 2 μM PEI | 1 μM PEI | 0.5 μM PEI | 0.25 μM PEI | 0.125 μM PEI |
| 0.0078M Suc | 0.0078M Suc | 0.0078M Suc | 0.0078M Suc | 0.0078M Suc | 0.0078M Suc |
| 0.78125 mM Raf | 0.78125 mM Raf | 0.78125 mM Raf | 0.78125 mM Raf | 0.78125 mM Raf | 0.78125 mM Raf |
| 4 μM PEI | 2 μM PEI | 1 μM PEI | 0.5 μM PEI | 0.25 μM PEI | 0.125 μM PEI |

| | | | | |
|---|---|---|---|---|
| 0.5M Suc, | 0.5M Suc, | 0.5M Suc, | 0.5M Suc, | 0.5M Suc, |
| 50 mM Raf | 50 mM Raf | 50 mM Raf | 50 mM Raf | 50 mM Raf |
| 0.0625 μM PEI | 0.03125 μM PEI | 0.015625 μM PEI | 0.007813 μM PEI | 0.003906 μM PEI |
| 0.25M Suc | 0.25M Suc | 0.25M Suc | 0.25M Suc | 0.25M Suc |
| 25 mM Raf | 25 mM Raf | 25 mM Raf | 25 mM Raf | 25 mM Raf |
| 0.0625 μM PEI | 0.03125 μM PEI | 0.015625 μM PEI | 0.007813 μM PEI | 0.003906 μM PEI |
| 0.125M Suc | 0.125M Suc | 0.125M Suc | 0.125M Suc | 0.125M Suc |
| 12.5 mM Raf | 12.5 mM Raf | 12.5 mM Raf | 12.5 mM Raf | 12.5 mM Raf |
| 0.0625 μM PEI | 0.03125 μM PEI | 0.015625 μM PEI | 0.007813 μM PEI | 0.003906 μM PEI |
| 0.0625M Suc | 0.0625M Suc | 0.0625M Suc | 0.0625M Suc | 0.0625M Suc |
| 6.25 mM Raf | 6.25 mM Raf | 6.25 mM Raf | 6.25 mM Raf | 6.25 mM Raf |
| 0.0625 μM PEI | 0.03125 μM PEI | 0.015625 μM PEI | 0.007813 μM PEI | 0.003906 μM PEI |
| 0.0312M Suc | 0.0312M Suc | 0.0312M Suc | 0.0312M Suc | 0.0312M Suc |
| 3.125 mM Raf | 3.125 mM Raf | 3.125 mM Raf | 3.125 mM Raf | 3.125 mM Raf |
| 0.0625 μM PEI | 0.03125 μM PEI | 0.015625 μM PEI | 0.007813 μM PEI | 0.003906 μM PEI |
| 0.0156M Suc | 0.0156M Suc | 0.0156M Suc | 0.0156M Suc | 0.0156M Suc |
| 1.56 mM Raf | 1.56 mM Raf | 1.56 mM Raf | 1.56 mM Raf | 1.56 mM Raf |
| 0.0625 μM PEI | 0.03125 μM PEI | 0.015625 μM PEI | 0.007813 μM PEI | 0.003906 μM PEI |
| 0.0078M Suc | 0.0078M Suc | 0.0078M Suc | 0.0078M Suc | 0.0078M Suc |
| 0.78125 mM Raf | 0.78125 mM Raf | 0.78125 mM Raf | 0.78125 mM Raf | 0.78125 mM Raf |
| 0.0625 μM PEI | 0.03125 μM PEI | 0.015625 μM PEI | 0.007813 μM PEI | 0.003906 μM PEI |

Samples were freeze-dried by the VirTis Advantage freeze-dryer for approximately 3 days. Samples were frozen at minus 40° C. for 1 hour before a vacuum was applied, initially at 200 milliTorre. Shelf temperature and vacuum were adjusted throughout the process and the condenser was maintained at minus 42° C. Step 8 was extended until the samples were stoppered before releasing the vacuum. The drying cycle used is shown below:

| | Shelf temp | Time | | Vacuum |
|---|---|---|---|---|
| Step | (° C.) | (mins) | Ramp/Hold | (milliTorre) |
| 1 | −45 | 15 | H | 200 |
| 2 | −32 | 600 | R | 200 |
| 3 | −20 | 120 | R | 200 |
| 4 | −10 | 120 | R | 200 |
| 5 | 0 | 120 | R | 200 |
| 6 | 10 | 120 | R | 200 |
| 7 | 20 | 120 | R | 200 |
| 8 | 20 | 1250 | H | 400 |

Following freeze-drying, glass vials were stoppered under vacuum and transferred to MaxQ 4450 incubator (Thermo Scientific) for heat challenge at 45° C. for 1 week. Following incubation, samples were prepared for the L929 assay. Specifically, the samples were reconstituted in sterile distilled water.

### L929 Assay for Assessment of TNFα Neutralisation

Antibody activity was measured using an anti-TNFα neutralisation assay. For this, L929 cells (mouse C3H/An connective tissue) were used. A suspension of 3.5×10⁵ cells per ml was prepared in 2% FBS in RPMI, and 100 μl of the cell suspension was added to each well of a 96 well plate and incubated overnight at 37° C., 5% CO₂. In a separate 96 well plate, neutralisation of the recombinant TNFα was set up by adding 50 μl of 2% FBS in RPMI to each well. 50 μl of the control rat anti-human TNFα antibody (Caltag) at a concentration of 10 μg/ml was added to columns 3-12. In the next row, reconstituted anti-TNFα antibody from freeze-dried product was also added at a concentration of 10 μg/ml.

A 1:2 dilution was carried out. 50 μl of recombinant human TNFα (Invitrogen) was added to well columns 2-12. The resulting antibody cytokine mixture was incubated for 2 hours at 37° C. Following incubation 50 μl per well of the antibody cytokine solution was transferred to the corresponding well of the plate containing the L929 cells. 50 μl of 0.25 μg/ml actinomycin was added to each well.

Plates were incubated for 24 hours at 37° C., 5% CO₂ in a humidified incubator. A fresh stock of 5 ml of MTT solution at 5 μg/ml was made up in PBS. 20 μl MTT solution was added to each well. The cells were then incubated (37° C., 5% CO₂) for 3-4 hours for the MTT to be metabolized. Following incubation, the media was discarded and the wells were dried.

The formazan product was resuspended in 100 μl DMSO, placed on a shaking table for 5 minutes to thoroughly mix the formazan into the solvent. The plate was read on a synergy HT plate reader and the optical density read at 560 nm. The background at 670 nm was then subtracted to give the final O.D.

### 3. Results

The results are shown in FIG. 6. This experiment sets out a matrix of optimisation for excipient concentrations by varying sugar concentrations and PEI concentrations. A high O.D. corresponds to good antibody stabilisation and reflects an effective neutralisation of the TNFα by the anti-TNFα antibody.

Following a week's challenge at 45° C., higher concentrations of Suc/Raf appeared to provide increased protection following heat challenge, as shown in FIG. 6. Additionally higher concentrations of PEI used in this experiment also provided increased protection when used in combination with higher concentrations of sugars.

### Example 7

### Stabilisation of Anti-TNFα Antibody

### 1. Materials

Same as Example 6.

### 2. Method

A sucrose solution was prepared by adding 10 g sucrose to 10 ml PBS in a 50 ml falcon tube to give a stock concentration of 1.8M. The solution was gently heated in a microwave to assist dissolution. A raffinose solution was prepared by adding 2.5 g raffinose to 5 ml PBS in a 50 ml falcon tube to give a stock concentration of 0.63M. The solution was heated in a microwave to allow complete dissolution. Once fully dissolved, a sugar mix was prepared by adding 4 ml raffinose solution to 16 ml sucrose solution.

A PEI solution was prepared by dissolving 1 g of PEI into 50 ml PBS giving a concentration of 0.167 mM based on Mn. Further dilutions of PEI solution were prepared in PBS.

Freeze-dried PBS controls were prepared with antibody lot 477758B and all other samples prepared with antibody lot 555790A. Samples were prepared for freeze-drying by adding 100 μl sugar mix, 100 μl PEI solution and 100 μl anti-TNFα antibody to glass vials. The final sugar and PEI concentrations of these samples are shown below. PFD=prior to freeze-drying; FD=freeze-dried.

| | Sucrose | Raffinose | PEI |
|---|---|---|---|
| Sample ID | conc (M) | conc (M) | conc (μM) |
| PFD PBS | 0 | 0 | 0 |
| PFD Sug | 0.24 | 0.021 | 0 |
| FD PBS | 0 | 0 | 0 |
| FD Sug | 0.48 | 0.042 | 0 |
| FD Sug + PEI | 0.48 | 0.042 | 2.78 |
| 2.78 μM | | | |
| FD Sug + PEI | 0.48 | 0.042 | 0.278 |
| 0.28 μM | | | |

Samples were vortexed and freeze-dried using the VirTis Advantage freeze-dryer (Biopharma Process Systems) as described in Example 6. On completion of drying samples were stoppered and capped. Sets of samples were analysed after 1 week's heat treatment at 60° C.

Freeze-dried and heat treated samples were re-suspended in 150 μl water. Samples were transferred to HPLC glass vials. 100 μl injections were compared by size exclusion HPLC (mobile phase of PBS at ambient temperature) measuring absorbance at 280 nm (flow rate of 0.3 ml/min, approx 1200 psi). Peak areas were determined.

### 3. Results

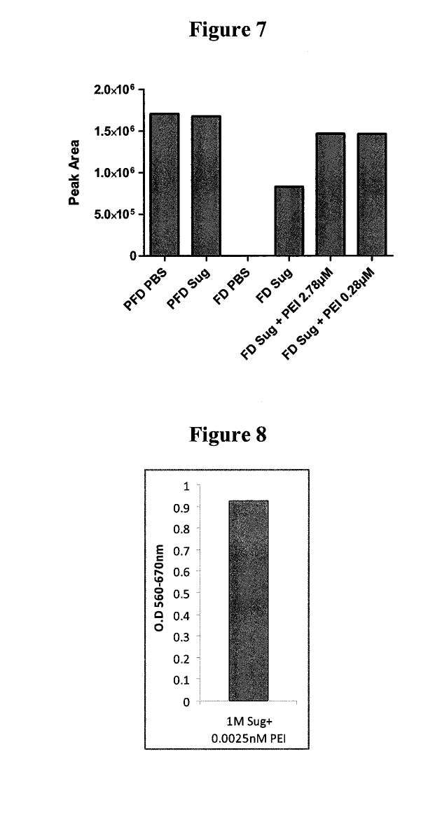

The results are shown in FIG. 7. No antibody was measured when freeze-dried in PBS. A significant amount of anti-TNFα antibody was lost when freeze-dried in sugars alone. A much greater amount of anti-TNFα antibody was measured when the antibody was freeze-dried with sugars and PEI.

### Example 8

### Stabilisation of Anti-TNFα Antibody

Following the procedures of Example 6, a PBS sample of the anti-TNFα antibody was prepared containing 0.9M sucrose, 0.1M raffinose and 0.0025 nM PEI. The sample was freeze-dried as described in Example 6. The sample was then heat-treated at 45° C. for 2 weeks. The heat-treated sample was reconstituted RPMI with 2% FBS. TNFα neutralisation was assessed in the L929 assay described in Example 6. The result is shown in FIG. 8. Good antibody stabilisation had been achieved.

### Example 9

### Stabilisation of Influenza Haemagglutinin

### 1. Materials

### Polyethyleneimine (P3143, Mn 60,000)

### Sucrose (Sigma)

### Raffinose (Fluka)

Dulbecco's phosphate buffered saline (PBS) (Sigma)
Glass vials (Adelphi glass)
Rubber stoppers (Adelphi glass)
UV transparent 96 well microtitre plates (Costar®)
Maxisorb 96 well ELISA plates (Nunc)
Citric acid (Sigma)
Rabbit anti-sheep Ig's HRP conjugate (AbCam)
30% H₂O₂ solution (Sigma)
Orthophenylenediamine (OPD) tablets (Sigma)

### H₂SO₄ (Sigma)

Polyclonal monospecific sheep anti H1 antibody (Solomon Islands) (NIBSC)
Polyoxysorbitan monolaurate (Tween 20) (Sigma)
Non-fat skimmed milk powder (Marvel)
Bromelain solubilised purified influenza haemagglutinin (HA) from X31 (H3N2)

### 2. Method

Preparation of Samples

1×57 μg vial of the influenza HA protein was reconstituted with 475 μl sterile distilled water (SDW) to give a stock concentration of 120 μg/ml. This stock was then further diluted 1/4 into SDW and then 1/6 into PBS or an excipient mixture comprising a combination of sucrose, raffinose and PEI, and further sterile distilled water. This resulted in a final concentration of HA of 5 μg/ml in an excipient comprising final concentrations of 1M sucrose/100 mM raffinose/16.6 nM PEI (based on Mn).

200 μl aliquots of these solutions were placed into 5 ml vials for freeze-drying (FD). Lyophilisation and secondary drying was carried out in a VirTis Advantage freeze-dryer using the protocol described in Example 6. After freeze-drying, one of the freeze-dried samples in excipient was thermally challenged at 80° C. in a water bath for 1 hour. All samples were then allowed to equilibrate to ambient temperature, the freeze-dried samples were reconstituted with 200 μl SDW and all samples were titrated in two-fold dilution series from an initial concentration of 1 μg/ml by ELISA as described below.

### ELISA Protocol

50 μl of each sample diluted in PBS was added to appropriate wells of a Maxisorb 96 well ELISA plate (Nunc). The plate was tapped to ensure even distribution over the well bases, covered and incubated at 37° C. for 1 hour. A blocking buffer was prepared consisting of PBS, 5% skimmed milk powder and 0.1% Tween 20. The plate was washed three times by flooding with PBS, discarding the wash and then tapping dry.

A 1 in 200 dilution of sheep anti H1 antibody (polyclonal monospecific sheep anti H1 antibody, Solomon Islands, NIBSC) in blocking buffer was prepared and 50 μl added to each well. The plate was covered and incubated at 37° C. for one hour. The plates were then washed three times in PBS.

A 1 in 1000 dilution of rabbit and sheep IgG, IgA and IgM was prepared in blocking buffer. 50 μl of this solution was then added to each well. The plates were then covered and incubated at 37° C. for one hour. The plates were then washed three times in PBS.

A substrate/OPD solution was then prepared by adding OPD (orthophenylenediamine) to a final concentration of 0.4 μg/ml in pH 5.0 citrate/phosphate buffer. 50 μl of a 0.4 μg/ml 30% H₂O₂ solution was then added to each assay well and the plate was incubated at ambient temperature for 10 minutes. The reaction was then stopped by the addition of 50 μl per well of 1M H₂SO₄ and the absorbents read at 490 nm.

### 3. Results

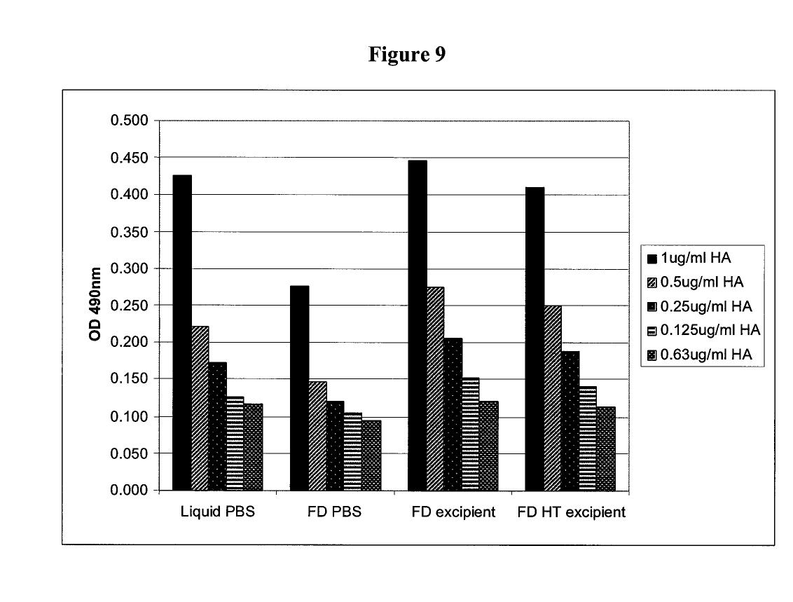

The results are shown in FIG. 9. Liquid PBS represents the control samples of HA in PBS alone. Substantially more HA was detected by ELISA in the freeze-dried HA samples containing the sucrose, raffinose and PEI excipient (FD excipient and FD HT excipient) than in the freeze-dried samples without excipient (FD PBS).

### Example 10

### Preservation of Luciferase

### 1. Method

Luciferase stock was purchased from Promega Corporation (code E1701) and consisted of 1 mg of purified protein at a concentration of 13.5 mg/ml, correlating to 2.13×10⁻⁴ M using an approximate molecular weight of 60 kDa. The stock was thawed and refrozen (untouched, without addition of any excipients) at −45° C. as 4 μL aliquots. These aliquots were subsequently used for all experiments.

Luciferin was purchased from Promega Corporation as a kit that also included ATP (code E1500). This kit shall henceforth be referred to as luciferin reagent and consisted of pairs of vials that required mixing before use. One vial contained a lyophilised powder and the other 10 ml of a frozen liquid. To produce stocks, these vials were mixed and then refrozen as 1 ml aliquots at −20° C. in standard 1.5 ml Eppendorf tubes. Vials and reconstituted luciferin reagent were stored at −20° C. in an opaque box and only removed under conditions of near-darkness.

Excipients (described below) and bovine serum albumin (BSA) were dissolved or diluted into PBS so as to minimise deviation of actual PBS concentration across the PBS buffers used. BSA stock was made up at 100 mg/ml and subsequently diluted to give a working concentration of 1 mg/ml. Wherever used to dilute luciferase, PBS buffer was always supplemented with 1 mg/mL BSA; luciferase was not exposed to any solution unless it was supplemented with 1 mg/mL BSA.

A fixed ratio of sucrose:raffinose (sugar mix or “sm”) was used throughout all experiments, but the final concentration of this ratio was varied. The final concentrations of sugars and PEI (Sigma P3143, Mn 60,000) used in this experiment are shown below.

A sucrose solution was prepared by adding 32 g sucrose powder to 32 ml PBS in a 50 ml falcon tube to give a final volume of 52 ml, correlating to a final concentration of 61.54%. The solution was gently heated in a microwave to assist initial solvation but thereafter stored at 4° C. Raffinose solution was prepared by adding 4 g raffinose to 8 ml PBS in a 50 ml falcon tube to give a final volume of 10.2 ml corresponding to a final concentration of 39.2%. The solution was heated in a microwave to allow complete solvation. Once fully dissolved, the raffinose solution would precipitate if stored alone for any length of time at room temperature or at 4° C.

To produce the final sugar mix, the sucrose and raffinose solutions described above were mixed in a 4:1 ratio. In practice, 32 ml sucrose solution was mixed with 8 ml raffinose solution. Once composed, sugar mix was stored indefinitely at 4° C. and suffered no precipitation.

The luciferase assay involved the mixing of various concentrations of luciferase with an undiluted aliquot of luciferin reagent in black opaque 96 well plates. The initial (linear) phase of this luminogenic reaction was then immediately quantified by a luminometer. As per the manufacturer's recommendation, luciferase samples were of a 100 μl volume and luminescence was initiated by addition of 100 μl of luciferin reagent. All steps involving luciferin reagent were conducted in near-darkness.

To compensate for inevitable background noise and to assure confidence, each sample was assayed (in triplicate) at multiple concentration points that were expected to generate a linear response. Due to rapid signal decay only three samples were assayed at one time. These corresponded to the triplicate preparations of each concentration point. Once read, triplicate samples corresponding to the next concentration point were then prepared and assayed. The following five concentration points were assayed for each sample:

### 6×10⁻¹⁰ M

### 5×10⁻¹⁰ M

### 4×10⁻¹⁰ M

### 3×10⁻¹⁰ M

### 2×10⁻¹⁰ M.

### Detailed Description of the Protocol

Luciferase has an extremely high specific activity that necessitates serial dilution prior to assay. Since luciferase is extremely fragile, such dilution is best done immediately prior to assay. Therefore, at the start of each experiment, one 4 μl aliquot of untouched stock luciferase at 2.21×10⁻⁴ M was removed from −45° C. storage and immediately placed on ice before being rapidly diluted with 880 μl ice-cold PBS to give a concentration of 1×10⁻⁶ M.

To achieve the desired working concentration of luciferase, further serial dilutions were then prepared, as described next. 100 μl of the freshly-prepared 1×10⁻⁶ M luciferase solution was added to 900 μl ice-cold PBS to give 1 ml at 1×10⁻⁷ M. 100 μl of this solution was then added to 900 μl ice-cold PBS to give 1 ml at 1×10⁻⁸ M. Between 20 μl and 60 μl of this solution was then added into 1 ml ice-cold PBS to give the five final stock solutions to be diluted tenfold to give the five working concentrations shown above (i.e. the final stocks were at 2 to 6×10⁻⁹ M). 10 μl of these stocks was diluted to a final assay volume of 100 μl (with or without excipients) using PBS with 1 mg/mL BSA to make up the volume to 100 μl.

All samples, including aliquots to be freeze-dried and freeze-dried aliquots that had been resuspended prior to assay, were always of 100 μl volume. Irrespective of excipient content or concentration, all 100 μl aliquots contained a final BSA concentration of 1 mg/ml. Sugar mix and PEI were tested alone and together at various concentrations (from 0 to 67% and 1×10⁻ to 1×10⁻⁷% respectively), and added either before or after freeze-drying. In all, the following combinations were tested (unless stated otherwise in the ‘Group’ column, excipients were added prior to freeze-drying):

| | Final Sugar Mix Concentration | | Final PEI Concentration | | |
|---|---|---|---|---|---|
| Group | % | Molar | % | Molar | |
| 1 | 67 | 0.96M Suc, 0.09M Raf | 0 | | |
| | 50 | 0.72M Suc, 0.07M Raf | | | |
| | 40 | 0.58M Suc, 0.05M Raf | | | |
| | 30 | 0.43M Suc, 0.04M Raf | | | |
| | 20 | 0.29M Suc, 0.03M Raf | | | |
| | 10 | 0.14M Suc, 0.01M Raf | | | |
| | 0 | 0M Suc, 0M Raf | | | |
| 2 | 67 | 0.96M Suc, 0.09M Raf | 1.7 × 10⁻¹ | 28.3 | μM |
| | 50 | 0.72M Suc, 0.07M Raf | 1.7 × 10⁻¹ | | |
| | 40 | 0.58M Suc, 0.05M Raf | 1.7 × 10⁻¹ | | |
| | 30 | 0.43M Suc, 0.04M Raf | 1.7 × 10⁻¹ | | |
| | 20 | 0.29M Suc, 0.03M Raf | 1.7 × 10⁻¹ | | |
| | 10 | 0.14M Suc, 0.01M Raf | 1.7 × 10⁻¹ | | |
| | 0 | 0M Suc, 0M Raf | 1.7 × 10⁻¹ | | |
| 3 | 67 | 0.96M Suc, 0.09M Raf | 1.7 × 10⁻¹ | 28.3 | μM |
| PEI added | 50 | 0.72M Suc, 0.07M Raf | 1.7 × 10⁻¹ | | |
| after FD | 40 | 0.58M Suc, 0.05M Raf | 1.7 × 10⁻¹ | | |
| | 30 | 0.43M Suc, 0.04M Raf | 1.7 × 10⁻¹ | | |
| | 20 | 0.29M Suc, 0.03M Raf | 1.7 × 10⁻¹ | | |
| | 10 | 0.14M Suc, 0.01M Raf | 1.7 × 10⁻¹ | | |
| | 0 | 0M Suc, 0M Raf | 1.7 × 10⁻¹ | | |
| 4 | 0 | | 1.0 × 10⁻⁰ | 167 | μM |
| | | | 1.0 × 10⁻¹ | 16.7 | μM |
| | | | 1.7 × 10⁻¹ | 28.3 | μM |
| | | | 1.0 × 10⁻² | 1.67 | μM |
| | | | 1.0 × 10⁻³ | 167 | nM |
| | | | 1.0 × 10⁻⁴ | 16.7 | nM |
| | | | 1.0 × 10⁻⁵ | 1.67 | nM |
| | | | 1.0 × 10⁻⁶ | 167 | pM |
| | | | 1.0 × 10⁻⁷ | 16.7 | pM |
| 5 | 67 | 0.96M Suc, 0.09M Raf | 1.0 × 10⁻⁰ | 167 | μM |
| | | | 1.0 × 10⁻¹ | 16.7 | μM |
| | | | 1.7 × 10⁻¹ | 28.3 | μM |
| | | | 1.0 × 10⁻² | 1.67 | μM |
| | | | 1.0 × 10⁻³ | 167 | nM |
| | | | 1.0 × 10⁻⁴ | 16.7 | nM |
| | | | 1.0 × 10⁻⁵ | 1.67 | nM |
| | | | 1.0 × 10⁻⁶ | 167 | pM |
| | | | 1.0 × 10⁻⁷ | 16.7 | pM |
| 6 | 67 | 0.96M Suc, 0.09M Raf | 1.0 × 10⁻⁰ | 167 | μM |
| sugar mix | | | 1.0 × 10⁻¹ | 16.7 | μM |
| added | | | 1.7 × 10⁻¹ | 28.3 | μM |
| after FD | | | 1.0 × 10⁻² | 1.67 | μM |
| | | | 1.0 × 10⁻³ | 167 | nM |
| | | | 1.0 × 10⁻⁴ | 16.7 | nM |
| | | | 1.0 × 10⁻⁵ | 1.67 | nM |
| | | | 1.0 × 10⁻⁶ | 167 | pM |
| | | | 1.0 × 10⁻⁷ | 16.7 | pM |

Samples were always composed in the following order: to 10 μl of luciferase stock (at 2 to 6×10⁻⁹ M) was added PBS (with 1 mg/ml BSA) then sugar mix then PEI, if either of the latter were indicated in the sample, otherwise they were excluded (see above table). In all cases final sample volume was made up to 100 μl with PBS containing 1 mg/ml BSA.

### Assay Procedure

Three 100 μl aliquots of the top concentration (6×10¹⁰ M luciferase) were pipetted into adjacent wells on a precooled black opaque 96 well plate. The 96 well plate was then placed into the luminometer reading tray. A multichannel pipette was then used to add and briefly mix 100 μl aliquots of luciferin reagent into the wells. Reading was then initiated immediately. After each reading the 96 well plate was immediately returned to ice to re-cool before the next reading. Data was then saved prior to the next triplicate samples being prepared and assayed.

### Resuspension of Freeze-Dried Samples

Samples for freeze-drying were prepared as 100 μl aliquots. Freeze-dried samples containing sugar mix were resuspended in a lesser volume (due to sugar mix contributing volume) to give a final volume of 100 μl. It was previously shown that 23.4 μl out of a volume of 100 μl was due to sugar mix when used at a concentration of 66.7% (data not shown). Accordingly, such samples were resuspended by the addition of 74.6 μl. The volume contributed by sugar mix in samples bearing less sugar mix was calculated from the above value and adjusted accordingly to result in a final volume of 100 μl.

### 2. Results

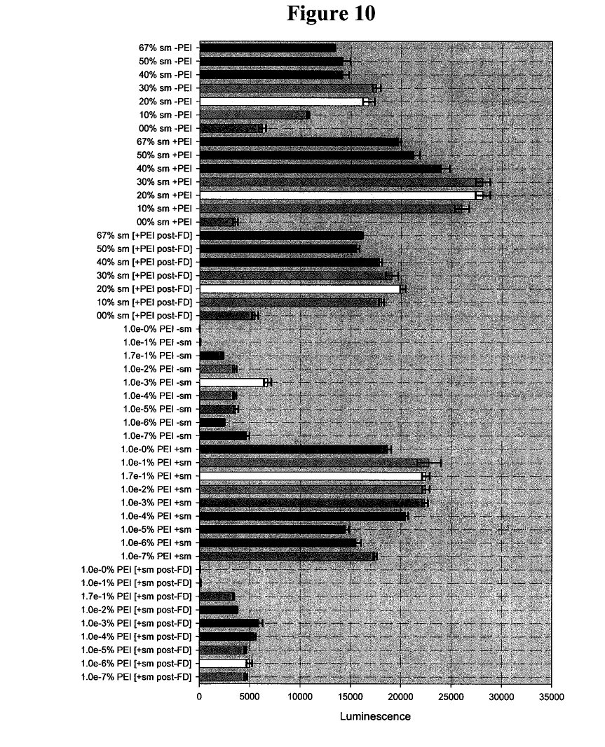

The results are shown in FIG. 10. Firstly, the optimal sugar mix (sm) concentration occurs from 20% (0.29M sucrose, 0.03M raffinose) to 30% (0.43M sucrose, 0.04M raffinose). This holds true both in the absence and the presence of PEI (first two data sets). The standard sugar mix concentration is 66.7% (0.96M sucrose, 0.09M raffinose). Optimal PEI concentration occurs at 1.0×10⁻³% PEI (167 nM based on Mn) in the absence of sugar mix (fourth data set) whilst in the presence of 66.7% sugar mix (fifth data set) it is maintained from 1.0×10⁻¹% through 1.0×10⁻³% PEI (16.7 μM to 167 nM based on Mn). Therefore, the lowest optimal excipient concentration is 20% sugar mix (0.29M sucrose, 0.03M raffinose) and 1.0×10⁻³% PEI (167 nM based on Mn).

The lyoprotectant effects of sugar mix and PEI are synergistic, peaking when both are added together (second and fifth datasets). This effect is most marked when comparing the protection afforded by PEI alone (fourth data set) to that observed when sugar mix is coincident (fifth data set). Most significantly, the presence of PEI provides extra lyoprotection compared to using sugar mix alone (first and second data sets respectively).

However, this synergistic effect is only observed when both components are added before lyophilisation. Adding either component after freeze-drying wholly negates its contribution relative to when that component was excluded: the excipients can protect but not resurrect.

### Example 11

### Preservation of β-Galactosidase

### Preparation of Samples

Excipients mixtures containing β-galactosidase were prepared according to the table below and vortexed. 10 units of β-galactosidase were added to each vial. 200 μl of the vortexed mixture was placed in each appropriately labelled 5 ml glass vials. PEI was obtained from Sigma (P3143, Mn 60,000).

| Vials Label | Suc | Raf | PEI |
|---|---|---|---|
| PBS | — | — | — |
| Sugar control | 1M Suc | 100 mM Raf | — |
| Sugar, PEI | 1M Suc | 100 mM Raf | 13.3 μM PEI |
| 13.3 mM | | | |

After vortexing, vials were frozen at −80° C. in freeze-dryer trays containing 30 ml water with rubber stoppers partially in. Frozen vials were transferred to the freeze-dryer stoppering shelf of the pre-cooled freeze-dryer (Thermo Fisher) and dried for 16 hours. The condenser chamber was −70° C. However there was no shelf control on the freeze-drying unit. Rubber stoppers were lowered fully into the vials under a vacuum before removing from freeze-dryer.

### Beta-Galactosidase Assay

Following freeze-drying, vials were reconstituted in 1 ml PBS. 100 μl of the resulting solution from each vial was added in duplicate (giving a total of 6 readings per excipient type) to each well of a flat bottom 96 well plate. The substrate x-gal was added as according to the manufacturer's instructions. Briefly, a stock solution of 20 mg/ml was made in DMSO and used at a 1 mg/ml working concentration. 100 μl was added to each well and the solution allowed to develop over 10 minutes. Following development, absorbance was measured at 630 nm on a synergy HT microplate. Background from blank wells was then subtracted from all the readings and results assessed using Prism Graphpad.

### Results

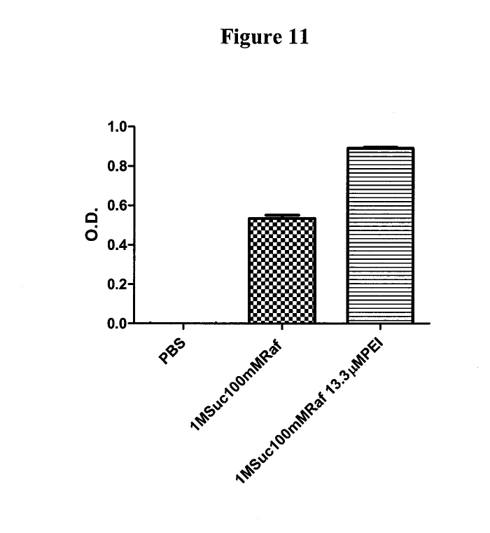

The results are shown in FIG. 11. This experiment examined the effect of freeze-drying β-galactosidase in the presence of sugar/PEI excipients. Following freeze-drying, β-galactosidase activity was high in sucrose/raffinose excipients compared to PBS. In sucrose/raffinose excipients containing PEI it was further enhanced.

### Example 12

### Preservation of Horse Radish Peroxidase (HRP)

Type IV horse radish peroxidase (HRP, Sigma-Aldrich) was diluted to 1 μg/ml in:

1. PBS alone

### 2. 1M Suc/100 mM Raf (Smix)

### 3. 1M Suc/100 mM Raf/16.6 nM PEI (SmixP)

The PEI was obtained from Sigma (P3143, Mn 60,000). The PEI concentration was based on Mn. 10×100 μl volumes of each of the above solutions were prepared in 5 ml freeze-drying vials. Five replicates of each solution were freeze-dried from minus 32° C. over a 3 day cycle on a VirTis Advantage laboratory freeze-dryer using the protocol described in Example 6.

One vial from each solution of the liquid and dried samples was placed at 4° C. while the rest were frozen to −20° C. Samples from each of the liquid and dry solutions were subjected to 2, 4, and 6 heat-freeze cycles by removing them from the −20° C. freezer and placing them in an incubator set at 37° C. for 4 hours before replacing them in the freezer for 20 hrs 2, 4 and 6 times. 1 vial of each was retained at −20° C. as a control.

When the cycling was completed all the samples, including the −20° C. and 4° C. maintained non-cycled controls were allowed to equilibrate to room temperature. The freeze-dried samples were then reconstituted with 100 μl/vial of deionised water at room temperature.

Triplicate 10 μl samples were removed from each vial into wells of a flat bottomed ELISA plate (Nunc Maxisorb). To each well was then added 50 ul of a chromogen/substrate solution containing 0.4 mg/ml orthophenylene diamine (OPD) and 0.4 μl/ml 30% hydrogen peroxide (H₂O₂). Colour was allowed to develop before the reaction was stopped by the addition of 50 μl/well of 1M sulphuric acid (H₂SO₄). Absorbance was measured at 490 nm on a BioTek Synergy HT spectrophotometer and plotted as optical density (OD).

### Results

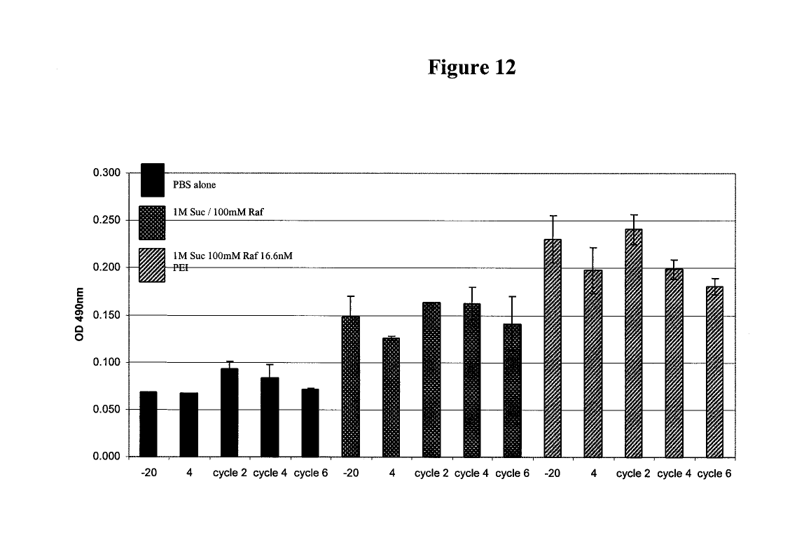

The results are shown in FIG. 12. For all treatments and storage conditions HRP activity is better maintained in the presence of sucrose/raffinose, either with or without PEI, than PBS alone. The pattern of HRP decay following consecutive heat/freeze cycles appears similar for all suspension media. However, the presence of sugars and especially sugars in combination with PEI, at the initial freeze-drying stage significantly reduces loss of HRP activity. Excipient-treated samples even following 6 heat/freeze cycles still maintained more HRP activity than unchallenged samples in PBS.

### Example 13

### Preservation of Alcohol Oxidase Activity

The aim of this experiment was to compare the efficacy of preservation of alcohol oxidase activity using the lactitol and PEI stabilizer according to Example 10 of WO 90/05182 (Gibson et al.) and using the present invention.

### Reagents

(All Reagents were Purchased from Sigma)
Sodium Dodecyl Sulphate; SDS—catalogue no. L4390
2,2′-azino-bis-3-ethylbenzthiazoline-6-suplhuric acid; ABTS—catalogue no. A1888
Methanol—catalogue no. 65543
Alcohol oxidase; AoX—catalogue no. A0438
Horseradish peroxidase—catalogue no. P8250

### Sugar mix (see Reagent Preparation)

Lactitol—catalogue no. L3250
PEI—catalogue no. P3143, Mn 60,000

### Storage and Preparation

All reagents except for SDS were made up fresh prior to each experiment. All reagents except for 2 mM ABTS and SDS were kept on ice during each experiment. 2 mM ABTS and 20% SDS were stored at room temperature.

The 20% working solution of SDS was prepared by adding 5 g SDS powder to 23.6 ml PBS solution to give a final volume of 25 ml. The powder was fully driven into solution by vortexing and then centrifuged to collapse surface foam.

1 g ABTS was mixed with 18.2 ml PBS to give a 100 mM solution. 1 ml of this solution was added into 50 ml PBS to give the working concentration of 2 mM.

A 1% working solution of methanol was used and was prepared by adding 500 μl methanol into 50 ml PBS.

A working solution of 10 U/ml alcohol oxidase (AoX) was used and was prepared by resuspending 100 U of enzyme into 10 ml PBS.

Horseradish peroxidase was used at 250 U/ml and was prepared by resuspending 5 kU enzyme into 20 ml PBS.

A 20% working solution of lactitol was prepared by dissolving 5 g lactitol into 25 ml PBS. PEI was added to the lactitol as required. The lactitol and PEI mixture was mixed with alcohol oxidase as required.

Sugar mix was composed of a 4:1 (by weight) ratio of sucrose (Sigma, 16104) to raffinose pentahydrate (Sigma, R0250) and was used at a concentration of either 67% or 20% in the final excipient mix. 67% sugar mix correlates to final concentrations of 0.96M sucrose and 0.09M raffinose whilst 20% sugar mix correlates to final concentrations of 0.29M sucrose and 0.03M raffinose.

Sucrose solution was prepared by adding 32 g sucrose powder to 32 ml PBS in a 50 ml falcon tube to give a final volume of 52 ml corresponding to a final concentration of 61.54%. The solution was gently heated in a microwave to assist initial solvation but thereafter stored at 4° C. Raffinose solution was prepared by adding 4 g raffinose to 8 ml PBS in a 50 ml falcon tube to give a final volume of 10.2 ml corresponding to a final concentration of 39.22%. The solution was heated in a microwave to allow complete solvation. Once fully dissolved, the raffinose solution would precipitate if stored alone for any length of time at room temperature or at 4° C.

To produce the final sugar mix, the sucrose and raffinose solutions described above were mixed in a 4:1 ratio. In practise, 32 ml sucrose solution was mixed with 8 ml raffinose solution. Once composed, sugar mix was stored indefinitely at 4° C. and suffered no precipitation. PEI was added to the sugar mix as required. The mixture of the sugar mix and PEI was mixed with alcohol oxidase as detailed below.

### Sample Preparation for Drying and Freeze-Drying

All samples were prepared and assayed in duplicate. All samples were made up to 100 μl with PBS as required. The order the reagents were added, if present in a given sample, was always as follows: to alcohol oxidase stock at 10 U/ml was first added PBS, then sugar mix or lactitol, then PEI. The actual volume of alcohol oxidase added to each sample was 10 μl of 10 U/ml stock. The actual volume of sugar mix added to each sample was 20 μl (for 20% samples) or 67 μl (for 67% samples) of neat stock prepared as described above. The actual volume of lactitol added to each sample was 25 μl of 20% stock. The actual volume of PEI added to each sample was always 10 μl of a given stock concentration: 1% (167 μM) stock for Gibson 1 (G1) samples, 0.1% (16.7 μM) stock for Gibson 2 (G2) and Stabilitech 1 (S1) samples or 0.01% (1.67 μM) stock for Stabilitech 2 (S2) samples.

For identical samples being tested on different days, a single master mix was prepared and then sub-aliquoted to give the final 100 μl samples. Dried and freeze-dried samples were stored at 37° C. after drying until assay time. Controls were tested only on day 0 unless stated otherwise. The following samples were prepared and assayed:

| | | | Final | |
|---|---|---|---|---|
| | Condition | State | [AoX] | Final Composition of Excipients & Notes |
| Controls | No MeOH^{a} | Wet | 1 U/mL | Untouched enzyme (no excipients); ^{a}no |
| | | | | Methanol substrate added during assay: tests |
| | | | | background reading of assay |
| | Untouched | | | Untouched enzyme (no excipients); this |
| | | | | experiment is the positive control and global |
| | | | | activity reference |
| | Gibson 1 (G1) | | | 5% lactitol, 0.1% (16.7 μM) PEI |
| | Gibson 2 (G2) | | | 5% lactitol, 0.01% (1.67 μM) PEI |
| | Stabilitech 1 | | | 67% sugar mix (0.96M sucrose, 0.09M |
| | (S1) | | | raffinose), 0.01% (1.67 μM) PEI |
| | Stabilitech 2 | | | 20% sugar mix (0.29M sucrose, 0.03M |
| | (S2) | | | raffinose), 0.001% (167 nM) PEI |
| | Dry Stock | Dried | | no excipients; Gibson's positive control and |
| | | | | global activity reference |
| | FD Stock | Freeze- | | no excipients (negative control for this |
| | | dried | | experiment) |
| Tests | Gibson 1 (G1) | Dried | | 5% lactitol, 0.1% (16.7 μM) PEI |
| | Gibson 2 (G2) | | | 5% lactitol, 0.01% (1.67 μM) PEI |
| | Gibson 1 (G1) | Freeze- | | 5% lactitol, 0.1% (16.7 μM) PEI |
| | Gibson 2 (G2) | dried | | 5% lactitol, 0.01% (1.67 μM) PEI |
| | Stabilitech 1 | | | 67% sugar mix (0.96M sucrose, 0.09M |
| | (S1) | | | raffinose), 0.01% (1.67 μM) PEI |
| | Stabilitech 2 | | | 20% sugar mix (0.29M sucrose, 0.03M |
| | (S2) | | | raffinose), 0.001% (167 nM) PEI |

### Drying and Freeze-Drying

Drying was performed for 10 hours at 30° C. under 50% atmospheric pressure. Freeze-drying was performed as standard using the following program on a VirTis Advantage freeze-dryer:

| | Shelf temp | Time | | Vacuum |
|---|---|---|---|---|
| Step | (° C.) | (mins) | Ramp/Hold | (milliTorre) |
| 1 | −32 | 120 | H | 80 |
| 2 | −32 | 1250 | H | 80 |
| 3 | −32 | 380 | H | 80 |
| 4 | 25 | 600 | R | 80 |
| 5 | 25 | 400 | H | 10 |
| 6 | 20 | 300 | R | 10 |

### Sample Assay

For wet control samples, 1 μl alcohol oxidase, excipients and up to 90 μl PBS were taken into a glass drying/freeze-drying vial to give a final volume of 100 μl. Dried and freeze-dried samples were instead resuspended to a final volume of 100 μl PBS. 2.8 ml 2 mM ABTS was then added to each vial. 10 μl peroxidase was then added to each vial. Vials were then briefly vortexed.

The colorimetric reaction was then initiated by addition of 10 μl 1% MeOH to each vial. Samples were taken every 5 minutes up to 55 min and added into wells of a 96 well plate. Plates were prepared in advance and contained 75 μl 20% SDS in each well to quench the reaction. Plates were read at 405 nm after the final time point. Enzyme activity was assessed by following the rate of reaction (defined as the quotient of the change in absorbance with respect to time). Blanking was not performed since gradients are effectively self-blanking.

### Results

The results are shown in FIG. 13. The activity of wet, dried and freeze-dried alcohol oxidase in the presence and absence of excipients is shown:
D0 to D16: days incubated at 37° C. (for dried and freeze-dried samples);
No MeOH: no methanol added (negative control);
wet: samples stored and tested without desiccation (i.e. fresh);
FD: freeze-dried;
D: dried;
G1&G2: excipient mix conditions Gibson 1 & 2 respectively according to Example 10 of WO 90/05182; and
S1 and S2: excipient mix conditions Stabilitech 1 and 2 respectively according to the present invention.

Only G1 exhibited significant negative effects towards activity (independent of and prior to any drying) whilst G1 and G2 display the greatest attenuation of activity in the wet state independent of and prior to any drying. Freeze-dried S1 provided the greatest level of protection. G1 and G2 provided significantly better protection when freeze-dried than when dried (but still not as good as S1). Thus the protocol of the present invention worked considerably better than the protocol described for the excipient mixes containing PEI in Example 10 of WO 90/05182 (Gibson et al.).

For at least the first 2 weeks, freeze-dried S1 stored at 37° C. displayed essentially the same activity profile as wet untouched stock stored at 4° C. Therefore, freeze-dried S1 stored even at 37° C. did not attenuate activity relative to cold wet storage. Furthermore, the rate of activity loss decays over the 2 week time course indicating that a majority of the activity may persist during long-term storage. This characteristic is absent from the best result described in WO 90/05182 (Gibson et al.) (freeze-dried G2) which had decayed to background by day 5.

Dried G1 and freeze-dried G1 and G2 provided essentially zero protection. The findings that G1 induced precipitation in the pre-desiccation wet state and that both G1 and G2 provided very wanting lyoprotection do not support the view that the excipients or protocol in WO 90/05182 (Gibson et al.) provide a good level of protection.

Freeze-dried S2 provided intermediate protection relative to freeze-dried S1 but unlike freeze-dried G2, this protection was stable throughout the entire 2 week test period.

Drying or freeze-drying in the absence of excipients totally precluded detectable activity. This is in direct contrast to the observations made in WO 90/05182 (Gibson et al.). WO 90/05182 (Gibson et al.) even quotes all excipient protection efficiencies relative to the dried, excipient-free state. Since even WO 90/05182 (Gibson et al.) most likely suffered significant activity loss on drying with or without excipients, for this experiment it was felt that a fairer approach would be to quote results relative to wet, untouched (i.e. standard unadulterated) enzyme.

### Example 14

### Preservation of G-CSF

### 1. Materials

Human recombinant G-CSF (10 μg) (MBL JM-4094-10)

### 37% Formaldehyde (BDH 20910.294)

### 30% H₂O₂ (Riedel-dehaen 31642)

### Phospho-ERK1/ERK2 (T202/Y204)

### Cell-based ELISA kit (R&D SYSTEMS KCB 1018)

HL-60 cells (ECACC98070106)

### RPMI 1640 (Sigma R8758)

Poly-L-Lysine (0.01% solution) (Sigma P4707)
Trypan blue (SigmaT6146-5G)

### Penicillin/streptomycin (GIBCO 15070)

### PEI (Sigma P3143, Lot 127K0110; Mn 60,000)

### Suc (Sigma 16104, Lot 70040)

### Raf (Sigma R0250, Lot 039K0016)

### PBS (Sigma D8662, Lot 118K2339)

### Water (Sigma W3500, Lot 8M0411)

5 ml glass vials (Adelphi Tubes VCD005)
14 mm freeze drying stoppers (Adelphi Tubes FDIA14WG/B)
14 mm caps (Adelphi Tubes CWPP14)

### Foetal Bovine Serum (Sigma F7524)

### 2. Method

The following solutions were prepared:

| Solutions/Media | Preparation |
|---|---|
| 8% Formaldehyde | 2.6 ml of 37% formaldehyde in 9.4 ml of |
| | 1x PBS. |
| Total ERK1/ERK2 | Reconstituted with 110 μl of 1x PBS |
| (T202/y204) Antibody | |
| Primary Antibody | 100 μl of the phosphor-ERK1/ERK2 |
| Mixture | Antibody and 100 μl Total ERK1/ERK2 |
| | Antibody to 9.8 ml of Blocking Buffer. |
| Secondary Antibody | Add 100 μl of the HRP-conjugated antibody |
| Mixture | and 100 μl of the AP-conjugated antibody |
| | to 9.8 ml of Blocking Buffer |
| Substrate F1 | Add the content of the substrate F1 |
| | Concentrate vial (50 ul) to the 10 ml of |
| | F1 Diluent in the brown bottle. |
| 1 X Wash Buffer | Add 60 ml of Wash Buffer (5x) to the 240 |
| | ml of 1x PBS to prepare 1x wash buffer. |

### Preparation of Sugar Solutions

Sucrose solution was prepared by adding 32 g sucrose powder to 32 ml PBS in a 50 ml falcon tube to give a final volume of 52 ml correlating to a final concentration of 61.54%. The solution was gently heated in a microwave to assist initial solvation but thereafter stored at 4° C.

Raffinose solution was prepared by adding 4 g raffinose to 8 ml PBS in a 50 ml falcon tube to give a final volume of 10.2 ml corresponding to a final concentration of 39.2%. The solution was heated in a microwave to allow complete solvation.

To produce the final sugar mix, the sucrose and raffinose solutions described above were mixed in a 4:1 ratio. In practise, 32 ml sucrose solution was mixed with 8 ml raffinose solution. Once composed, sugar mix was stored at 4° C. and suffered no precipitation.

Preparation of PEI Solutions

6 g of PEI (50% w/v) was added to 500 ml PBS to make 6 mg/ml then diluted 1 in 10 to make 600 ug/ml. 600 μg/ml was then diluted to make 100 μg/ml solution of PEI. Serial 1 in 10 dilutions were prepared in PBS to a concentration of 0.01 μg/ml. For final concentrations of PEI refer to Table 2 below. Concentrations were calculated based on Mn.

Preparation of G-CSF to Mix with Excipients

10 μg of 98% purified recombinant human G-CSF was reconstituted in 1 ml of PBS and diluted to 0.2 ng/ml in PBS (1 in 50,000 dilution), with a starting concentration 10 μg/ml. The G-CSF was aliquoted in 15 μl in Eppendorf tubes and stored at −20° C. for further use.

First dilution: 10 μl of G-CSF at 10 μg/ml was added to 990 μl of PBS (1 in 100 dilutions). Second dilution: 10 μl of 1 in 100 dilutions of G-CSF was added to 4.99 ml of PBS (1 in 500 dilutions). For final concentrations of G-CSF refer to Table 2.

### Preparation of Excipients

Excipients were prepared according to Table 2. The final concentration of G-CSF was 0.2 ng/ml per vial. The final concentration of sucrose, raffinose and PEI are shown in Table 2. The excipients were vortexed to mix and 100 μl placed in each appropriately labelled 5 ml glass vial. Samples were freeze dried by the VirTis Advantage freeze dryer for approximately 3 days.

TABLE 2

| Vials* | Suc | Raf | PEI | |
|---|---|---|---|---|
| G-CSF (0.2 ng)-EXP/FD/HT/at 56° C. | 1M | 0.1M | 1.6 | μM |
| for 15 min, 1 h, 2 h and O/N | | | | |
| G-CSF (0.2 ng)-EXP/FD/HT/at 56° C. | 1M | 0.1M | 0.16 | μM |
| for 15 min, 1 h, 2 h and O/N | | | | |
| G-CSF (0.2 ng)-EXP/FD/HT/at 56° C. | 1M | 0.1M | 0.016 | μM |
| for 15 min, 1 h, 2 h and O/N | | | | |

*FD = Freeze-dried. HT = Heat-treated.

### Resuspension of Samples

Samples were prepared as 100 μl aliquots. Freeze-dried samples were resuspended in 100 μl of water.

### Day 1

The ELISA assay method described below was followed as general assay procedure of cell base assay's kit (R&D Systems).

### Tissue Culture

HL-60 cells were maintained in phenol red containing RPMI 1640 supplemented with 20% foetal bovine serum (FBS), Glutamine and Penicillin Streptomycin. HL-60 cells were passaged weekly and medium was replenished every 2-3 days.

The HL-60 (passage three) were transferred to a centrifuge tube and spun down at 1300 rpm, for 5 minutes at 4° C. The supernatant was poured off into a T-75 flask. The pellet was resuspended in 10 ml cold media.

200 μl of cell suspension was transferred into an Eppendorf tube by using a 5 ml pipette. 100 μl of cell suspension was added to 100 μl of trypan blue into another Eppendorf tube and mixed.

A haematocytometer was used for counting cells and the cell concentration was adjusted to 5×10⁵ cells/in 10 ml.

### Coating Plate

100 μl/well of 10 μg/ml Poly-L-Lysine was added to the microplate. The plate was covered with seal plate and incubated for 30 min, at 37° C. Poly-L-Lysine was removed from each well and washed 2 times with 100 μl of 1×PBS.

100 μl/well of HL-60 cell line (5×10⁵ cells in 10 ml) was added to the plate. The plate was covered and incubated at 37° C., 5% CO₂ overnight.

### Day 2

### Cell Stimulation

The test sample vials were reconstituted into 1000 of sterile water. The plate was washed 3 times with 100 μl of 1×PBS; each wash step was performed for five minutes. 90 μl/well of the completed RPMI media was added to the plate and then 10 μl/well of the reconstituted test samples were added to the plate. The plate was covered and incubated for 1 hour at 37° C. at 5% CO₂.

### Cell Fixation

An ELISA plate was washed as before and 1000/well of 8% Formaldehyde in 1×PBS was added to the plate. The plate was covered and incubated for 20 minutes at room temperature.

Formaldehyde solution was removed and the plate washed 3 times with 200 μl of 1× wash buffer, each wash step was performed for five minutes with gentle shaking.

Wash buffer was removed and 100 μl/well of Quenching Buffer was added to the plate. The plate was covered and incubated for 20 minutes at room temperature. Quenching Buffer was removed and the plate washed as before and 100 μl/well of Blocking Buffer was added to the plate. The plate was covered and incubated for 1 hour at room temperature.

### Binding of Primary and Secondary Antibodies

Blocking buffer was removed and the plate was washed as before and 100 μl/well of the primary antibody mixture was added to the plate. The plate was covered and incubated overnight at 4° C.

### Day 3

Primary antibody mixture was removed and the plate was washed as before and 100 μl/well of the secondary antibody mixture was added to the plate. The plate was covered and incubated for 2 hours at room temperature.

### Fluorogenic Detection

Secondary antibody mixture was removed and the cells were washed as before then followed by 2 washes with 200 μl of 1×PBS. Each wash step was performed for five minutes with gentle shaking.

1×PBS was removed and 75 μl/well of substrate (labelled substrate F1 by RnD Systems) to the plate and the plate was covered and wrapped with foil then incubated for 1 hour at room temperature. 75 μl/well of the second substrate (labelled substrate F2 by RnD Systems) was added to the plate and the plate covered and wrapped with foil and incubated for 40 minutes at room temperature.

Development of ELISA plate

The ELISA plate was read twice, the first read was with excitation at 540 nm and emission at 600 nm. The plate was then read at excitation at 360 nm and emission at 450 nm by fluorescence plate reader.

The results were expressed as the absorbance readings at 600 nm represent the amount of phosphorylated ERK1/ERK2 in the cells, while reading at 450 nm represent the amount of total ERK1/ERK2 in the cells.

### Data Analysis

The mean OD_{600 nm} was calculated of duplicate wells for each sample. The mean OD_{450 nm} was calculated of duplicate wells for each sample. The mean absorbance at 600 nm and at 450 nm was calculated and plotted against test samples (excipient and without excipient) containing recombinant human G-CSF.

### 3. Results

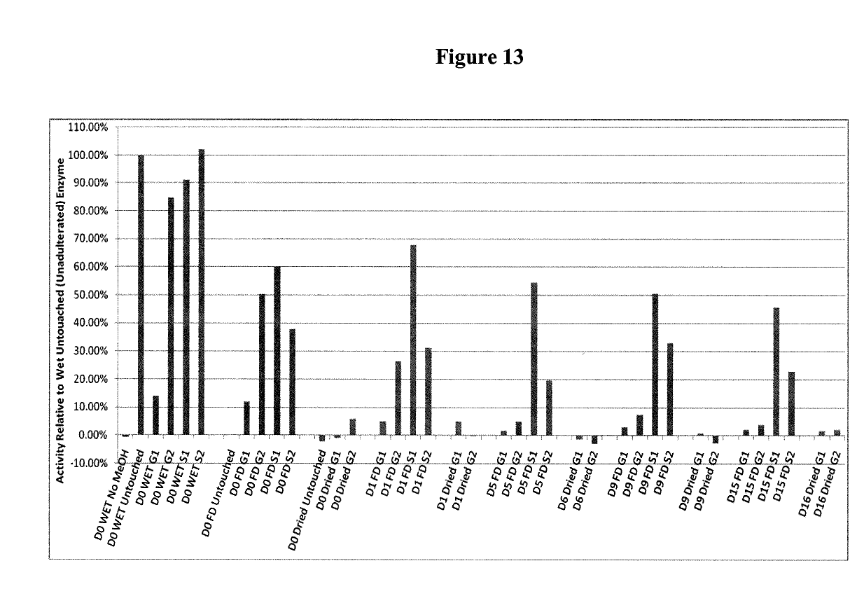

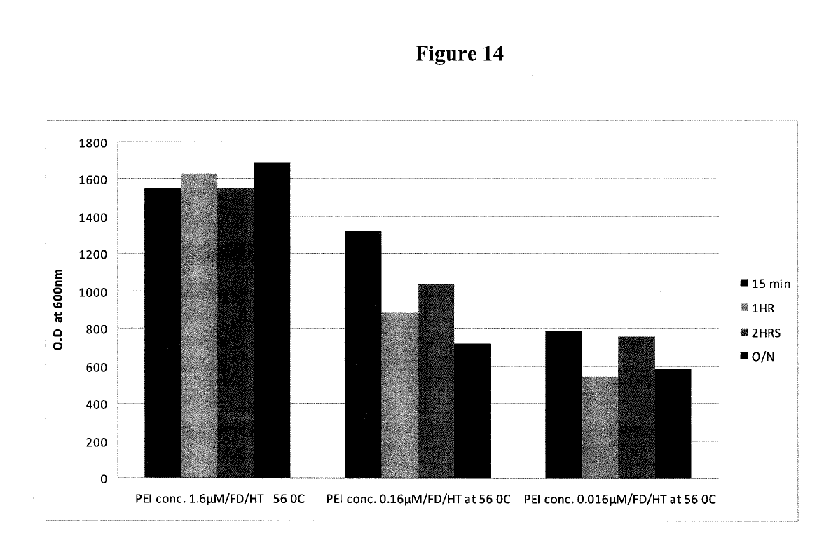

The results are shown in FIG. 14. The results indicate that mixing G-CSF with the excipient which contains 1.6 μM, 0.16 μM or 0.016 μM PEI, together with sucrose and raffinose, followed by freeze drying and heat treatment resulted in a higher level of phosphorylated ERK1/ERK2.

The results confirmed that the freeze-drying excipients appeared to protect G-CSF against heat inactivation. As clearly shown in a cell-based bioassay, the level of phosphorylated ERK1/ERK2 activation by G-CSF is highest when the excipient comprising PEI and sugars is used. A positive result in this assay also confirms that G-CSF freeze-dried with high level of PEI had greater efficacy. These results suggest that sugars in combination with high levels of PEI have greater thermal protection of G-CSF at 56° C.

### Example 15

### Stabilisation of IgM antibody

### 1. Methods

### Preparation of Test Samples

Stocks of IgM purified from human serum (Sigma catalogue no. 18260) were obtained in buffered aqueous solution (0.05M Tris-HCl, 0.2M sodium chloride, pH 8.0, containing 15 mM sodium azide) and stored at 4° C. Aliquots of 10 μm IgM were mixed with an excipient composed of PBS, an excipient composed of 1M sucrose and 0.1M raffinose in PBS, and an excipient composed of 1M sucrose, 0.1M raffinose and 16.7 μM (1 mg/ml) PEI (Sigma catalogue no. 18260) also in PBS in a total volume of 50 μl.

Each formulation treatment was made up in duplicate. Samples were lyophilised on a VirTis Advantage Freeze Dryer using the protocol described in Example 6. This program took 3 days after which time the samples were capped. On day 3 of the experiment, samples were placed in an environmental chamber with a cycling temperature regime of 12 hours at 37° C. followed by 10 hours at −20° C. with an hour of ramping between each temperature.

On day 10 of the experiment, after 7 days of temperature cycling, samples were reconstituted in 1 ml PBS and analysed by Size Exclusion HPLC.

### HPLC Analysis

Test samples and standards were run on a silica based size exclusion column (TSK-Gel Super SW3000 SEC Column, 4.6 mm internal diameter, 30 cm length) and compatible guard column (TSK-Gel PW_{XL} Guard Column, 6.0 mm internal diameter, 4.0 cm length). The mobile phase was PBS (pH 7.0). Injection volumes of 100 μl were applied to the column with a flow rate of 0.3 ml/min at ambient temperature with a run time of 25 minutes. Primary detection of IgM and degradants was by measuring maximum absorbance between 195 and 290 nm.

### Transformation of Data

Standards of known IgM concentration (10-0.1 μg/ml) were made up in 150 μl PBS. These standards were also analysed by Size Exclusion HPLC and the height of the major peak was measured (retention time of between 14.5 and 16.1 minutes) and a least squares regression line produced to describe the data. This equation was used to estimate the IgM concentration in test samples and this was then converted to percentage recovery of IgM relative to the known starting concentration (10 μg/ml).

### 2. Results

### Standard Curves for the Estimation of IgM Content

Size Exclusion HPLC and detection of components using a photodiode array could detect as little as 0.05 μg (0.5 μg/ml) of IgM. In the range 10-0.5 μg/ml IgM a good linear correlation was observed between IgM concentration and major peak height (R²=0.993). Least squares regression analysis was used to describe the fit (y=9136.7x+1659.2, where y=peak height and x=IgM concentration) and the equation generated used to estimate IgM concentration in test samples.

### Thermostability of IgM

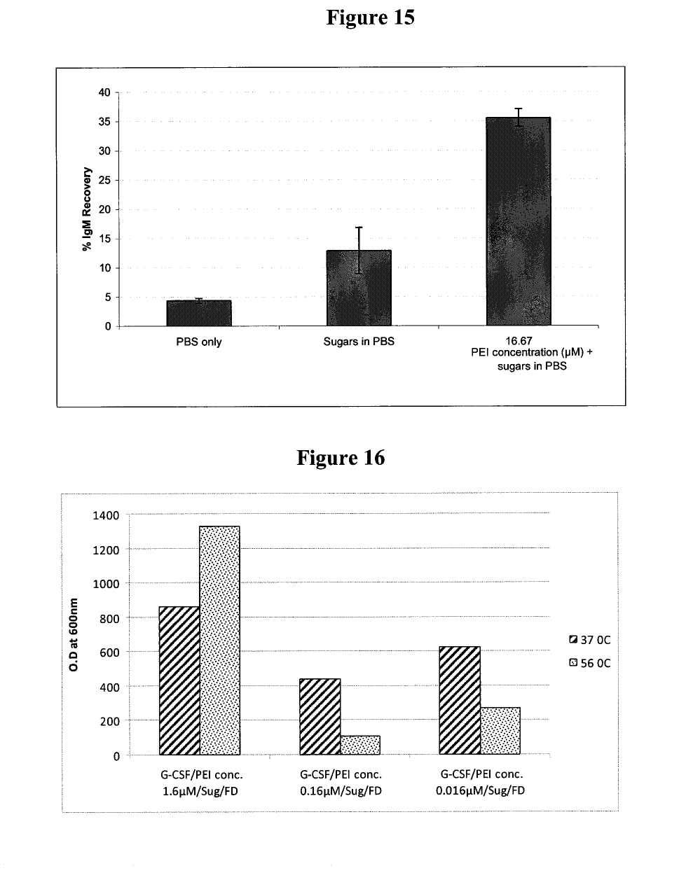

Size exclusion HPLC can only give an estimate of the percent recovery of native IgM. The recovery of IgM under the thermocycling conditions is quite poor, yielding less than 5% of starting IgM after only 7 days. The addition of sugars (1M sucrose and 0.1M raffinose) more than doubled this recovery (12.9%). However, recovery remained poor. Addition of 16.67 μM PEI markedly enhanced the efficacy of the excipients as thermoprotection, as there was 35.6% recovery of IgM (see FIG. 15).

### Example 16

### Preservation of G-CSF

Materials were as in Example 14. Excipients were set up as in Table 3 to allow for incubation at 56° C. as well as 37° C. for 1 week following freeze drying. After heat challenge, phosphorylation levels of ERK 1/2 were assayed as in Example 14.

TABLE 3

| | | | |
|---|---|---|---|
| Vials Label | Suc | Raf | PEI |

| | | | | |
|---|---|---|---|---|
| 1 - G-CSF at 0.2 ng/mL/EXP/FD/ | 1M | 0.1M | 1.6 | μM |
| at 56° C./for 1 week | | | | |
| 2 - G-CSF at 0.2 ng/mL/EXP/FD/ | 1M | 0.1M | 0.16 | μM |
| at 56° C./for 1 week | | | | |
| 3 - G-CSF at 0.2 ng/mL/EXP/FD/ | 1M | 0.1M | 0.016 | μM |
| at 56° C./for 1 week | | | | |
| 1 - G-CSF at 0.2 ng/mL/EXP/FD/ | 1M | 0.1M | 1.6 | μM |
| at 37° C./for 1 week | | | | |
| 2 - G-CSF at 0.2 ng/mL/EXP/FD/ | 1M | 0.1M | 0.16 | μM |
| at 37° C./for 1 week | | | | |
| 3 - G-CSF at 0.2 ng/mL/EXP/FD/ | 1M | 0.1M | 0.016 | μM |
| at 37° C./for 1 week | | | | |

The results are shown in FIG. 16. The results indicate that G-CSF with an excipient containing 1.6 μM, 0.16 μM or 0.016 μM PEI, together with sucrose and raffinose, protects and stabilises G-CSF during freeze drying and heat challenge. The highest level of protection of G-CSF, as reflected in higher levels of ERK 1/2 phosphorylation, was seen when sugars were used in combination with a PEI final concentration of 1.6 μM. This was evident at both 37° C. and 56° C. incubations.

### Example 17

### Materials

Chemical

| Supplier | ProductCode | Lot No. |
|---|---|---|
| Dulbecco's phosphate | Sigma D8662 | RNBB4780 |
| buffered saline | | |
| Polyethyleneimine | Sigma 482595 | 05329KH |
| Raffinose | Sigma R0250 | 050M0053 |
| Sucrose | Sigma 16104 | SZB90120 |
| Tween 20 | Sigma P1379 | 087K0197 |
| Skimmed milk powder | Marvel | |
| TMB chromogen | Invitrogen SB02 | 72764382A |
| Sulphuric acid | Sigma 25,8105 | S55134-258 |

Biological

| Supplier | Product Code |
|---|---|
| Bivalent F(ab′)2 | AbDSerotec AbD09357.4 |
| Antigen - IgG2b kappa | AbDSerotec PRP05 |
| Goat anti human HRP | AbDSerotec STAR12P |
| Rabbit anti mouse HRP | AbDSerotec STAR13B |
| Normal mouse serum | Sigma M5905 |

Other

| Manufacturer | Product Code |
|---|---|
| 2 ml Eppendorf tubes | VWR 16466-058 |
| ELISA immunoplates | NUNC 439454 |

Equipment

| Manufacturer | Equipment No. |
|---|---|
| Forma 900 series −80° C. freezer | Thermofisher EQP#015 |
| ATL-84-1 Atlion Balance | Acculab EQP#088 |
| +56° C. Incubator | Binder EQP#010 |
| Med Line +4° C. fridge | Liebherr EQP#019 |
| +40° C. incubator | Binder EQP#009 |
| Synergy HT Microplate reader | Biotek EQP#027 |

### Methods

The bivalent F(ab′)2 was thermally challenged in the presence of various concentrations of excipients and assayed at different points. An ELISA assay was used to assess the residual F(ab′)2 activity—this was used to measure the extent of damage sustained.

Preparation of and Thermal Challenge of Bivalent F(ab)2 in a Liquid Setting with Excipients

Bivalent F(ab′)2 in PBS, was removed from storage at −80° C. and allowed to thaw at room temperature. To determine the protective properties of the excipients in a liquid setting, 900 μl of each formulation with an antibody concentration of 4 μg/ml was made up—this quantity is sufficient to assay three separate timepoints. See Table 4 for details of each formulation.

TABLE 4
details of excipient formulations

| Abbreviation | Description | Suc | Raff | PEI | |
|---|---|---|---|---|---|
| -SR/-P (×2) | no Suc/Raff/PEI, PBS only | — | — | — | |
| LoSR/-P | Low [Suc/Raff], no PEI, in PBS | 0.1M | 0.01M | — | |
| HiSR/-P | High [Suc/Raff], no PEI, in PBS | 1M | 0.1M | — | |
| LoSR/LoP | Low [Suc/Raff], low [PEI], in PBS | 0.1M | 0.01M | 1.67 | nM |
| LoSR/MedP | Low [Suc/Raff], medium [PEI], in PBS | 0.1M | 0.01M | 16.67 | nM |
| LoSR/HiP | Low [Suc/Raff], high [PEI], in PBS | 0.1M | 0.01M | 166.67 | nM |
| HiSR/LoP | High [Suc/Raff], low [PEI], in PBS | 1M | 0.1M | 1.67 | nM |
| HiSR/MedP | High [Suc/Raff], medium [PEI], in PBS | 1M | 0.1M | 16.67 | nM |
| HiSR/HiP | High [Suc/Raff], high [PEI], in PBS | 1M | 0.1M | 166.67 | nM |

Two vials of the -SR/-P (control) formulation were set up - one was stored at +4° C. (as a positive control - no damage expected) and the second was placed at +56° C. with the other formulations (as a negative control; this formulation was not expected to remain stable and retain activity after 24 hours at an elevated temperature).

### Assay of Bivalent F(ab′)2 Activity

The activity of the Bivalent F(ab′)2 was assayed by ELISA. Antigen (Rat IgG2b-kappa) diluted to 0.5 μg/ml in PBS was coated 100 μl/well in row A to G of a 96-well plate, as well as two extra wells in row H for the +4° C. control conditions. Normal mouse serum at a 1:400,000 dilution was also added to two wells of row H as a positive control. These controls were used to normalise data later. Plates were incubated for 18 hours at +4° C. then washed three times with PBS containing 0.05% Tween 20 (wash buffer).

Plates were dried by blotting onto a paper towel. This method of blotting was used in every wash step. Plates were blocked for 1.5 hours with PBS containing 5% skimmed milk powder and 0.05% Tween 20. Plates were washed three times with wash buffer before adding the samples.

After incubation at thermal challenge (or +4° C. for control vial), the F(ab′)2 formulations were removed from incubator/fridge and 250 μl was removed from each. This was diluted 1:2 with wash buffer. Each diluted sample was added to the plate in duplicate and was diluted 2-fold down the plate (final concentrations ranging from 2 μg/ml to 0.0625 μg/ml). A condition with no bivalent F(ab′)2 was also included to measure the background signal. The plates were incubated at room temperature for 1.5 hours after which time the plates were washed five times with wash buffer.

A goat anti-human HRP conjugated antibody was diluted 1:5000 in wash buffer and 100 μl added to all the wells containing bivalent F(ab′)2. Rabbit anti-mouse HRP conjugate was diluted 1:1000 in wash buffer and 100 μl added to the wells containing the normal mouse serum control. The plates were incubated at room temperature for 1.5 hours then washed five times with wash buffer.

100 μl of TMB stabilised chromogen was added to each well and was allowed to react for 10 minutes at room temperature, after which time 100 μl 200 mM sulphuric acid was added to stop the reaction. The plates were read at 450 nm using Synergy HT Microplate reader.

### Statistical Analysis

The average and standard deviation was taken for each duplicate and the data points plotted as a line graph or as a bar graph at a designated F(ab′)2 concentration.

### Results

Activity of Bivalent F(ab)2 Fragments after Thermal Treatment at +56° C. for 24 Hours

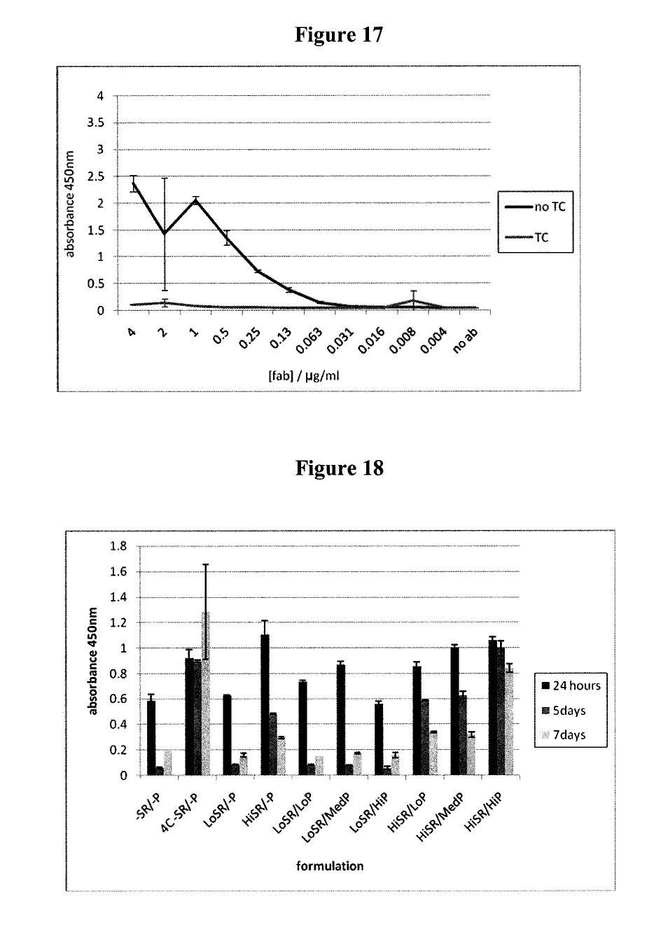

In a preliminary study, stock F(ab′)2 (as supplied by AbD Serotec—concentration 0.73 mg/ml) was stored at +56° C. to assess initial stability at elevated temperatures. The antibody was found to be extremely heat labile with little activity remaining after 24 hours at 56° C., providing an excellent starting point for testing the ability of the excipients to stabilise this antibody (FIG. 17).

Activity of Bivalent F(ab)2 Fragments after Thermal Treatment at +56° C. with and without Excipients

The bivalent F(ab′)2 was thermally challenged in the presence of various concentrations of the excipients and assayed at different points (see FIG. 18). After 24 hours storage at +56° C. most samples maintained the majority of their F(ab′)2 activity (when compared to the control sample stored a +4° C.), however after 5 days samples formulated with low or no sugar, the residual F(ab′)2 activity dropped to between 21% and 33% when compared to the activity remaining after 24 hours. Samples which contain high sugar concentration retained at least 44% activity after 5 days storage at +56° C.—this was increased to between 63% to 94% with the addition of PEI.

The final timepoint was taken at 7 days thermal challenge at +56° C. The control sample had not lost any activity, as expected. The samples which were formulated with low or no sugar had lost the majority of their F(ab′)2 activity. Samples which contained high sugar concentration maintained at least 27% of the 24 hour sample, this was increased to 79% when 10 μg/ml of PEI was added.

### CONCLUSION

Samples stored at +4° C. for seven days do not sustain any loss in F(ab′)2 activity, as expected. Samples which contain low sugar concentration, with or without PEI, lose the majority of F(ab′)2 activity after 5 days at +56° C. The most protective formulations contained high sugar concentration, and the addition of 10 μg/ml PEI significantly increases the protection. After 7 days TC, all low sugar concentration samples lost the majority of F(ab′)2 activity, whereas those which contained high sugar concentration and PEI still maintained a significant level of F(ab′)2 activity.

All publications, patent applications, patents, and other references cited in this specification are incorporated herein by reference in their entireties.

1: A method for preserving a polypeptide comprising:
(i) providing an aqueous solution of one or more sugars, a polyethyleneimine and said polypeptide; and
(ii) drying the solution to form an amorphous solid matrix comprising said polypeptide.

2: The method according to claim 1 wherein the concentration of polyethyleneimine is 25 μM or less based on the number-average molar mass (Mₙ) of the polyethyleneimine and the sugar concentration or, if more than one sugar is present, total sugar concentration is greater than 0.1M.

3: The method according to claim 2 in which
(a) the Mₙ of the polyethyleneimine is between 20 and 1000 kDa and the concentration of the polyethyleneimine is between 0.001 and 100 nM based on the Mₙ, and/or
(b) the Mₙ of the polyethyleneimine is between 1 and 10000 Da and the concentration of the polyethyleneimine is between 0.0001 and 1004 based on the and/or
(c) the said concentration of polyethyleneimine is 20 μM or less or less than 500 nM, and/or
(d) the said concentration of polyethyleneimine is 0.025 nM or more or 0.1 nM or more, and/or
(e) the said concentration of polyethyleneimine is between 0.1 nM and 5 μM or between 0.1 nM and 200 nM.

4: The method according to claim 2 in which
(a) the sugar concentration, or total sugar concentration, is between 0.5 and 2M; and/or
(b) the sugar is sucrose, stachyose, raffinose or a sugar alcohol, or
(c) two or more sugars are present in said aqueous solution, or
(d) two or more sugars are present in said aqueous solution and wherein sucrose is present with another sugar; the concentration of sucrose relative to the other sugar is at a ratio of molar concentrations of between 3:7 and 9:1; and the concentration of polytheyleneimine based on Mₙ in step (i) is between 0.0025 nM and 5 μM, and/or
(e) the sugars are sucrose and raffinose.

5: The method according to claim 2 in which
(a) the solution is freeze-dried in step (ii), or
(b) the polypeptide is a hormone, growth factor, peptide or cytokine, or
(c) the polypeptide is a tachykinin peptide, a vasoactive intestinal peptide, a pancreatic polypeptide-related peptide, an opioid peptide or a calcintonin peptide, or
(d) the polypeptide is an antibody or antigen-binding fragment thereof, or
(e) the polypeptide is an antibody or antigen-binding fragment thereof in which the antibody or antigen-binding fragment is a monoclonal antibody or fragment thereof, or
(f) the polypeptide is an antibody or antigen-binding fragment thereof in which the antibody or antigen-binding fragment is a chimeric, humanized or human antibody, or fragment thereof, or
(g) the polypeptide is an antibody or antigen-binding fragment thereof in which the antibody or antigen-binding fragment is a chimeric, humanized or human antibody, or fragment thereof which is an IgG1, IgG2 or IgG4 or antigen-binding fragment thereof, or
(h) the polypeptide is an antibody or antigen-binding fragment which is capable of binding to: (i) tumour necrosis factor α (TNF-α), interleukin-2 (IL-2), interleukin-6 (IL-6), glycoprotein CD33, CD52, CD20, CD11a, CD3, RSV F protein, HER2/neu (erbB2) receptor, vascular endothelial growth factor (VEGF), epidermal growth factor receptor (EGFR), anti-TRAILR2 (anti-tumour necrosis factor-related apoptosis-inducing ligand receptor 2), complement system protein C5, α4 integrin or IgE, or (ii) epithelial cell adhesion molecule (EpCAM), mucin-1 (MUC1/Can-Ag), EGFR, CD20, carcinoembryonic antigen (CEA), HER2, CD22, CD33, Lewis Y or prostate-specific membrane antigen (PMSA).

6: The method according to claim 2 in which the polypeptide is
(a) an enzyme, or
(b) an enzyme which is an oxidoreductase, a transferase, a hydrolase, a lyase, an isomerase or a ligase, or
(c) an enzyme selected from an α-galactosidase, β-galactosidase, luciferase, serine proteinase, endopeptidase, caspase, chymase, chymotrypsin, endopeptidase, granzyme, papain, pancreatic elastase, oryzin, plasmin, renin, subtilisin, thrombin, trypsin, tryptase, urokinase, amylase, xylanase, lipase, transglutaminase, cell-wall-degrading enzyme, glucanase, glucoamylase, coagulating enzyme, milk protein hydrolysate, cell-wall degrading enzyme, coagulating enzyme, lysozyme, fibre-degrading enzyme, phytase, cellulase, hemicellulase, protease, mannanase or glucoamylase, or
(d) a vaccine immunogen, or
(e) a vaccine immunogen which is a full-length viral or bacterial protein, glycoprotein or lipoprotein; or a fragment thereof.

7: The method according to claim 2 which further comprises providing the resulting dried amorphous solid matrix in the form of a powder in a sealed vial, ampoule or syringe.

8: A method for preserving a vaccine immunogen comprising:
(i) providing an aqueous solution of one or more sugars, a polyethyleneimine and said vaccine immunogen; and
(ii) drying the solution to form an amorphous solid matrix comprising said vaccine immunogen.

9: The method according to claim 8 wherein the concentration of polyethyleneimine is 25 μM or less based on the number-average molar mass (Mₙ) of the polyethyleneimine and the sugar concentration or, if more than one sugar is present, total sugar concentration is greater than 0.1M.

10: The method according to claim 9 in which
(a) the Mₙ of the polyethyleneimine is between 20 and 1000 kDa and the concentration of the polyethyleneimine is between 0.001 and 100 nM based on the Mₙ and/or
(b) the Mₙ of the polyethyleneimine is between 1 and 10000 Da and the concentration of the polyethyleneimine is between 0.0001 and 10 μM based on the Mₙ, and/or
(c) the said concentration of polyethyleneimine is 20 μM or less or less than 500 nM, and/or
(d) the said concentration of polyethyleneimine is 0.025 nM or more or 0.1 nM or more, and/or
(e) the said concentration of polyethyleneimine is between 0.1 nM and 5 μM or between 0.1 nM and 200 nM.

11: The method according to claim 9 in which
(a) the sugar concentration, or total sugar concentration, is between 0.5 and 2M, and/or
(b) the sugar is sucrose, stachyose, raffinose or a sugar alcohol, and/or
(c) two or more sugars are present in said aqueous solution, and/or
(d) sucrose is present with another sugar; the concentration of sucrose relative to the other sugar is at a ratio of molar concentrations of between 3:7 and 9:1; and the concentration of polytheyleneimine based on Mₙ in step (i) is between 0.0025 nM and 5 μM, and/or
(e) the sugars are sucrose and raffinose.

12: The method according to claim 9 in which the solution is freeze-dried in step (ii).

13: The method according to claim 9 in which
(a) the vaccine immunogen is a subunit vaccine, conjugate vaccine or toxoid, or
(b) the vaccine immunogen is a subunit vaccine in which the subunit vaccine immunogen is derived from a viral surface protein or viral capsid protein.

14: The method according to claim 8 further comprising providing the resulting dried amorphous solid matrix in the form of a powder in a sealed vial, ampoule or syringe.

15: A dry powder comprising preserved polypeptide or vaccine immunogen, obtained by the method as defined in claim 1.

16: A dry powder comprising preserved polypeptide or vaccine immunogen, obtained by the method as defined in claim 8.

17: A preserved product comprising a polypeptide or vaccine immunogen, one or more sugars and polyethylenimine, which product is in the form of an amorphous solid.

18: A method of preparing a vaccine comprising a vaccine immunogen, which method comprises:
(a) providing an aqueous solution of one or more sugars, a polyethyleneimine and said vaccine immunogen wherein the concentration of polyethyleneimine is 15 μM or less based on the number-average molar mass (Mₙ) of the polyethyleneimine and the sugar concentration or, if more than one sugar is present, total sugar concentration is greater than 0.1M; and
(b) optionally adding an adjuvant, buffer, antibiotic and/or additive to the admixture; and
drying the solution to form an amorphous solid matrix comprising said vaccine immunogen.

19: A vaccine comprising a preserved product as defined in claim 15 and optionally an adjuvant.

20: A vaccine comprising a preserved product as defined in claim 16 and optionally an adjuvant.

21: A vaccine comprising a vaccine obtained by the method of claim 18 and optionally an adjuvant.

22: A sealed vial, ampoule or syringe containing a dry powder as defined in claim 15.

23: A sealed vial, ampoule or syringe containing a dry powder as defined in claim 16.

24: A sealed vial, ampoule or syringe containing a preserved product as defined in claim 17.

25: A sealed vial, ampoule or syringe containing a vaccine as defined in claim 19.

26: A sealed vial, ampoule or syringe containing a vaccine as defined in claim 20.

27: A sealed vial, ampoule or syringe containing a vaccine as defined in claim 21.

28: A method for preserving a polypeptide prior to drying comprising:
(i) providing an aqueous solution of one or more sugars, a polyethyleneimine and said polypeptide; and
(ii) storing the solution for up to five years in a sealed container.

29: A method according to claim 28, which further comprises:
(iii) drying the solution to form an amorphous solid matrix comprising said polypeptide.

30: A method according to claim 28, in which (a) the solution is stored in a refrigerator, or (b) the solution is stored in a freezer.

31: A bulk aqueous solution of one or more sugars, a polyethyleneimine and a polypeptide, which solution is provided in a sealed container and is stored prior to drying in a refrigerator or freezer.

32: A solution according to claim 31 in which the bulk aqueous solution has a volume of 0.1 to 100 litres.
